(19) United States
(12) Patent Application Publication
(10) Pub. No.: US 2014/0072581 A1
(43) Pub. Date: Mar. 13, 2014

(54) Immunoglobulin Constructs Comprising Selective Pairing of the Light and Heavy Chains

(71) Applicant: Zymeworks Inc., Vancouver (CA)

(72) Inventors: Surjit Bhimarao DIXIT, Richmond (CA); Dunja UROSEV, Vancouver (CA); Gordon Yiu Kon NG, Vancouver (CA); Igor Edmondo Paolo D'ANGELO, Port Moody (CA)

(73) Assignee: Zymeworks Inc., Vancouver (CA)

(21) Appl. No.: 13/949,166

(22) Filed: Jul. 23, 2013

Related U.S. Application Data
(60) Provisional application No. 61/857,652, filed on Jul. 23, 2013.
(60) Provisional application No. 61/674,820, filed on Jul. 23, 2012.

(51) Int. Cl. C07K 16/18; C07K 16/00

(52) U.S. Cl. CPC C07K 16/18; C07K 16/00
USPC 424/172.1; 530/387.3; 530/387.1; 530/389.6; 530/389.1; 530/389.7; 435/320.1; 435/69.6

(57) ABSTRACT

Disclosed herein is an isolated immunoglobulin construct comprising a first monomeric polypeptide comprising a first single chain Fv polypeptide connected to a first constant domain polypeptide; and a second monomeric polypeptide comprising a second single chain Fv polypeptide, connected to a second constant domain polypeptide; each said constant domain polypeptide comprising at least one each of a CL domain, a CH1 domain, a CH2 domain and a CH3 domain or fragments, variants or derivatives thereof; and wherein said first and second constant domain polypeptide form a Fc region.

### CROSS-REFERENCE TO RELATED APPLICATIONS

This application claims the benefit of U.S. Application Ser. No. 61/674,820, filed Jul. 23, 2012; and U.S. Application Ser. No. 61/857,652, filed Jul. 23, 2013, each of which is hereby incorporated by reference in its entirety.

### FIELD OF THE INVENTION

The field of the invention is the rational design of immunoglobulin constructs for custom development of biotherapeutics.

### BACKGROUND OF THE INVENTION

Therapeutic monoclonal antibodies (Mabs) are widely used to treat human diseases. However, targeting only one antigen usually is insufficient in indications like oncology, and tumors progress after a latency period. A generic methodology to convert existing antibodies into an IgG-like bispecific format would greatly facilitate the clinical development of bispecific antibodies. Since the early days of antibody engineering there has been a broad interest in the generation of such antibodies that can bind two different targets simultaneously. Bispecific antibodies such as tetravalent IgG-single-chain variable fragment (scFv) fusions, catumaxomab, a trifunctional rat/mouse hybrid bispecific epithelial cell adhesion molecule-CD3 antibody, the bispecific CD19-CD3 scFv antibody blinatumomab, “dual-acting Fab” (DAF) antibodies, tetravalent bispecific formats such as the IgG-like dual-variable-domain antibodies (DVD-Ig), have been described in the art. Each of these approaches has limitations such as immunogenicity, poor pharmacokinetic properties, or loss of effector functions caused by the lack of a fragment crystallizable (Fc) region; also, they may tend to aggregate or may contain potentially immunogenic non-human domains. Most formats deviate significantly from the natural IgG protein architecture, or they cannot be applied for the preparation of stable bispecific IgG antibodies in a generic manner based on available antibodies.

### SUMMARY OF THE INVENTION

Provided herein are immunoglobulin constructs comprising a single chain Fab region (scFab) that comprises a variable region polypeptide (VH) from an immunoglobulin heavy chain, a variable region polypeptide (VL) from an immunoglobulin light chain, a constant region polypeptide (CL) from an immunoglobulin light chain, and a constant region polypeptide (CH1) from an immunoglobulin heavy chain; wherein VH and VL polypeptides are connected by a first linker to form a single chain Fv construct (scFv). In some embodiments, said CL and CH1 are connected by a second linker. In certain embodiments, the immunoglobulin construct has a sequence comprising VH-L1-VL-CL-L2-CH1, wherein L1 and L2 are first and second linkers. In certain embodiments, the immunoglobulin construct has a sequence comprising VH-L1-VL-L3-CL-L2-CH1, wherein L1, L2 and L3 are linkers. In an embodiment, the immunoglobulin construct has a sequence comprising VL-L4-VH-CH1-L5-CL, wherein L4 and L5 are linkers. Certain embodiments of such constructs are alternately referred to as Light Chain Inserts (LCI) herein.

In certain embodiments, each linker is a polypeptide comprising from about 1 to about 100 amino acids. In some embodiments, the linker comprises an amino acid sequence comprising amino acids selected from Gly (G), Ser (S) and Glu (E). In an embodiment, said linker is comprised of polypeptide of the general formula (Gly-Gly-Gly-Ser)n wherein n is an integer from 4 to 10.

Provided is an isolated immunoglobulin construct comprising: a single chain Fab region (scFab) that comprises: a variable region polypeptide (VH) from an immunoglobulin heavy chain, a variable region polypeptide (VL) from an immunoglobulin light chain, a constant region polypeptide (CL) from an immunoglobulin light chain, and a constant region polypeptide (CH1) from an immunoglobulin heavy chain; wherein said VH and CL are connected by a linker polypeptide, wherein said linker polypeptide exhibits a propensity to form a helical structure. In some embodiments, the single chain Fab polypeptide has a sequence comprising VL-CL-L8-VH-CH1; wherein L8 is said linker with a propensity to form a helical structure. In some embodiments, at least about 25% of the linker exists in helical form. In some embodiments, at least about 50% of the linker exists in helical form. In certain other embodiments, at least about 60% of the linker exists in helical form. In another embodiment, at least about 75% of the linker exists in helical form. In another embodiment, at least about 80% of the linker exists in helical form. In further embodiments, at least about 90% of the linker exists in helical form. In further embodiments, at least about 95% of the linker exists in helical form. In certain embodiment, the linker comprises multiple helical segments.

Provided are immunoglobulin constructs described herein wherein the linker polypeptide forms at least one of an alpha helix, a polyproline type I helix, a polyproline type II helix and a 3₁₀ helix. In some embodiments, the linker forms between about 1 turn to about 20 turns of a helix. In an embodiment, the linker forms between about 3 turn to about 5 turns of a helix. In an embodiment, the linker forms between about 2 turn to about 4 turns of a helix. In an embodiment, the linker forms between about 2 turn to about 10 turns of a helix. In some embodiments, the linker comprises at least one pair of amino acids that form helix stabilizing interactions. In an embodiment, the helix stabilizing interaction is at least one of a charge-charge interaction, a cation-pi interaction, a hydrophobic interaction and a size complimentary interaction.

Provided are isolated immunoglobulin constructs described herein, wherein said construct comprises at least one linker polypeptide with propensity to form a helix, and wherein said linker polypeptide comprises amino acids selected from Gly (G), Ser (S), Glu (E), Gln (Q), Asp (D), Asn (N), Arg (R), Lys (K), His (H), Val (V) and Ile (I). In certain embodiments, the linker polypeptide comprises amino acids selected from Met (M), Ala (A), Leu (L), Glu (E) and Lys (K). In an embodiment, the linker polypeptide comprises at least one Pro (P) residue. In certain embodiments, the linker has an amino acid sequence comprising at least one (Asp-Asp-Ala-Lys-Lys)n motif wherein n is an integer from 1 to 10.

Provided herein are immunoglobulin constructs comprising: a first polypeptide construct comprising a first scFab described herein; and a first heavy chain polypeptide comprising a first CH3 region; and a second polypeptide construct comprising a second heavy chain polypeptide comprising a second CH3 region, wherein at least one of said first and second heavy chain polypeptides optionally comprises a variant CH3 region that promotes the formation of a heterodimer. In some embodiments, said first and second polypeptide construct further comprising an antigen binding polypeptide construct. In an embodiment, the antigen binding polypeptide construct is at least one of an scFv or a scFab. In some embodiments, the scFab is an scFab described herein.

In some embodiments, the first and second heavy chain polypeptides form a heterodimeric Fc. In certain embodiments, the heterodimeric Fc comprises a variant immunoglobulin CH3 domain comprising at least one amino acid mutation. In certain embodiments, said at least one amino acid mutation promotes the formation of said heterodimeric Fc with stability comparable to a native homodimeric Fc. In an embodiment, the variant CH3 domain has a melting temperature (Tm) of about 73° C. or greater. In an embodiment, the heterodimeric Fc is formed with a purity of at least about 70%. In some embodiments, the heterodimeric Fc is formed with a purity of at least about 70% and the Tm is at least about 73° C. In another embodiment, the heterodimeric Fc is formed with a purity of at least about 75% and the Tm is about 75° C.

Provided is an immunoglobulin construct described herein, wherein at least one of said first and second heavy chain polypeptides further comprising a variant CH2 domain comprising amino acid modifications to promote selective binding to at least one of the Fcgamma receptors. In an embodiment, at least one of said first and second heavy chain polypeptides comprises a variant CH2 domain or hinge comprising amino acid modifications that prevents functionally effective binding to at least one of the Fcgamma receptors. In some embodiments, the Fc region is glycosylated. In some embodiments, the Fc region is aglycosylated. In an embodiment, the Fc region is fucosylated. In another embodiment, the Fc region is afucosylated.

In some embodiments is provided an immunoglobulin construct described herein, wherein said immunoglobulin construct is a multispecific immunoglobulin construct. In an embodiment, the immunoglobulin construct is bispecific.

Provided is an isolated immunoglobulin construct comprising: a first monomeric polypeptide comprising a first single chain Fv polypeptide connected by a linker to a first constant domain polypeptide; and a second monomeric polypeptide comprising a second single chain Fv polypeptide which is different from said first Fv polypeptide, connected by a linker to a second constant domain polypeptide; each said constant domain polypeptide comprising at least one each of a CL region, a CH1 region, and a CH3 region or fragments, variants or derivatives thereof; and wherein said CL and CH1 regions are connected by a linker, and wherein said first and second constant domain polypeptide form a Fc region. In some embodiments, the construct does not contain any CH2 domains. In some embodiments, the CH3 domain from said first constant domain polypeptide is different from the CH3 domain from said second constant domain polypeptide and said first and second constant domain polypeptide CH3 domains pair to form stable heterodimeric Fc.

In an embodiment is provided an isolated immunoglobulin construct comprising: a first monomeric polypeptide comprising a first scFab polypeptide fused to a first constant domain polypeptide; and a second monomeric polypeptide comprising a second scFab polypeptide which is different from said first Fab polypeptide, fused to a second constant domain polypeptide; wherein at least one of said first and second scFab polypeptides comprises a linker polypeptide with a propensity to form a helical structure; and wherein said first and second constant domain polypeptides form a heterodimeric Fc region comprising a variant immunoglobulin CH3 region comprising at least one amino acid mutation that promotes the formation of said heterodimer with stability comparable to a native homodimeric Fc.

Provided are immunoglobulin constructs described herein, wherein said construct binds at least one target antigen selected from CD3, CD19, HER2, Tissue factor and CD16a. In certain embodiments, are immunoglobulin constructs described herein that bind at least one antigen expressed by an immune cell, leukocyte, subendothelial cell or cancer cell. In certain embodiments, are immunoglobulin constructs described herein that bind an antigen expressed by a T cell. In some embodiments, the T cell is at least one of CD4+, CD8+ T cell, a cytotoxic T cell and a CD16a+ natural killer T cell. In certain embodiments, are immunoglobulin constructs described herein that bind an antigen expressed by a B cell.

In some embodiments, the B cell is CD19+, and a cancer cell. In certain embodiments, are immunoglobulin constructs described herein that bind an antigen expressed by a cancer cell such as HER2. In some embodiments, are immunoglobulin constructs described herein that bind an antigen expressed by a subendothelial cell or leukocyte such as Tissue Factor.

In an embodiment is provided an immunoglobulin construct described herein, wherein said construct can bind at least one T cell or Natural killer cell and at least one other cell that expresses an antigen. In an embodiment is provided an immunoglobulin construct described herein, wherein said construct can bind at least one T cell and at least one B cell. In some embodiments, the T cell is a human cell. In an embodiment, the T cell is a non-human, mammalian cell. In some embodiments, the immunoglobulin construct described herein binds an antigen expressed on a cell is associated with a disease. In some embodiments, the disease is a cancer. In an embodiment, the cancer is selected from a carcinoma, a sarcoma, leukaemia, lymphoma and glioma. In an embodiment, the cancer is at least one of a sarcoma, a blastoma, a papilloma and an adenoma. In some embodiments, the cancer is at least one of squamous cell carcinoma, adenocarcinoma, transition cell carcinoma, osteosarcoma and soft tissue sarcoma.

In some embodiments, the immunoglobulin construct described herein binds an antigen on at least one cell which is an autoimmune reactive cell. In some embodiments, the autoimmune reactive cell is a lymphoid or myeloid cell.

Provided herein is a pharmaceutical composition comprising an isolated immunoglobulin construct described herein; and a suitable excipient.

In an embodiment is a process for the production of a pharmaceutical composition described herein, said process comprising: culturing a host cell under conditions allowing the expression of an immunoglobulin construct as described herein; recovering the produced immunoglobulin construct from the culture; and producing the pharmaceutical composition.

Provided is a method of treating cancer in a mammal in need thereof, comprising administering to the mammal a composition comprising an effective amount of the pharmaceutical composition described herein. Also provided is a use of an immunoglobulin construct described herein in the treatment of cancer in a mammal in need thereof, comprising administering to the mammal a composition comprising an effective amount of the immunoglobulin construct described herein. In an embodiment the cancer is a solid tumor. In some embodiments, the solid tumor is one or more of sarcoma, carcinoma, and lymphoma. In an embodiment, the cancer is one or more of B-cell lymphoma, non-Hodgkin's lymphoma, and leukemia.

Provided is a method of treating an autoimmune condition in a mammal in need thereof, comprising administering to said mammal a composition comprising an effective amount of the pharmaceutical composition described herein. Also provided is a use of an immunoglobulin construct described herein in the treatment of an autoimmune disease, said use comprising providing a composition comprising an effective amount of the immunoglobulin construct described herein. In some embodiments, the autoimmune condition is one or more of multiple sclerosis, rheumatoid arthritis, lupus erytematosus, psoriatic arthritis, psoriasis, vasculitis, uveitis, Crohn's disease, and type 1 diabetes.

Provided is a method of treating an inflammatory condition in a mammal in need thereof, comprising administering to said mammal a composition comprising an effective amount of the pharmaceutical composition described herein. Also provided is use of an immunoglobulin construct in the treatment of an inflammatory condition in an individual, comprising providing to said individual an effective amount of an immunoglobulin construct described herein.

Provided herein is a kit comprising an immunoglublin construct as defined herein; and instructions for use thereof.

### BRIEF DESCRIPTION OF THE FIGURES

FIG. 1A-1B provide graphical representations of canonical IgG1 antibody structure (FIG. 1A) and an asymmetric bispecific antibody (FIG. 1B). In FIG. 1A, one of the two fragment antigen binding (Fab) arms is highlighted in the inset box. The light chain is represented by the gradient and the heavy chain by the [symbol] gradient. The disulphide link between the different chains is represented with dotted lines. The CH1 domain of the heavy chain leads into the linker followed by the CH2 and CH3 domains that are involved in pairing with the second heavy chain resulting in the Fc. While the molecule is symmetric down the axis between the two heavy chains in a regular monoclonal antibody like IgG1, the goal of an asymmetric bispecific antibody is to create an antibody like molecule involving the selective pairing of two different heavy and two different light chains. The asymmetric bispecific antibody is represented by the distinct chain patterns in FIG. 1B; the second heavy chain is represented by the [symbol] gradient while the second light chain is represented by the gradient.

FIG. 2 is a graphical representation of the design of a single-chain Fv polypeptide connected to a constant domain polypeptide. The figure shows that a linker is introduced between the C-terminus of VH and N-terminus of VL. Additionally, a second linker is introduced between the C-terminus of CL and N-terminus of CH1 domain. The linkers are selected such that they reconstitute the interdomain VL-VH geometry, resulting in native Fab like antigen binding.

FIG. 3 provides a graphical representation of a bispecific immunoglobulin construct comprising two monomeric polypeptides, each comprising a single-chain Fv polypeptide connected to a constant domain polypeptide.

FIG. 4 provides graphical representation of the design of a single-chain Fab wherein the light chain is fused to the N-terminus of the heavy chain, thereby expressing the Fab as a single chain comprising of the domains VL-CL-VH-CH1. The obligate VL and VH domains pair up, as do the CL and CH1 domains

FIG. 5 provides graphical representation of an asymmetric bispecific molecule based on the use of two different single-chain Fab segments engineered with the single chain Fab design wherein the Fab is a single-chain Fab comprising the domains VL-CL-VH-CH1.

FIG. 6A-6G depicts SDS-PAGE results following expression of 3 different scFabs (4D5, TF and NM3E) in the light chain insert (LCI) (6A-6C) and long linker (LL) formats (6D-6F). FIG. 6G represents SDS-PAGE gels of scFab NM3E2 with different linker inserts.

FIG. 7 shows an SDS-PAGE profile illustrating the monomeric single chain Fab species isolated by size exclusion chromatography from each preparation in reducing and non-reducing condition.

FIG. 8A-8B shows Antigen binding (ELISA). scFab (LL and LCI version of D3H44, v665 and 673) and control Fab (v696) binding to TF. scFab (LL and LCI version of 4D5, v654 and 656) and control Fab (v695) binding to HER2.

FIG. 9A-E shows Differential Scanning calorimetry (DSC) experiments performed on different variants. All DSC experiments were carried out using a GE or MicroCal VP-Capillary instrument.

FIG. 10 shows Benchtop stability assay of single chain Fab format. Left Panel: Day1; Centre Panel Day 3; Right Panel: Day 7. Results indicate that all single chain Fab samples do not re-multimerize, at the relatively dilute concentration used, during the week-long study.

FIG. 11. shows expression of monospecific bivalent scMabs (heterodimeric Fc).

FIG. 12A-12C shows SDS-PAGE analysis of variants, illustrating the benchtop stability assay of scMab.

FIG. 13 shows Expression and purification of bivalent bispecific scMabs (heterodimeric Fc) in CHO cell line.

FIG. 14A-14D show SEC profile and SPR sandwich assay of bispecific scMab(LL/LL).

FIG. 15A-15C show SEC profile and SPR sandwich assay of bispecific scMab (LCI/LCI).

FIG. 16A-16C show SEC and target binding profile of bispecific scMabs (LCI/LCI:1358; LCI/LL: 1359)

FIG. 17 shows SDS-PAGE analysis of D3H44 and 4D5 scFabs after scale-up and purification.

FIG. 18 shows SDS-PAGE expression analysis of CD3/CD19 bivalent, bi-specific scMabs. A, B, and C refer to the ratio of Chain A to Chain B used in the expression: Ratio A=Chain A/Chain B=1:1 A/B=50%/50%; Ratio B=Chain A/Chain B=2:1 A/B=66%/34%; Ratio C=Chain A/Chain B=1:2 A/B=34%/66%.

FIG. 19: Molecular model of the Light Chain Insert format. Linkers of diverse lengths connecting the VL and VL domains and CL and CH1 domains can be introduced. A cut can be introduced at one of the peptide bond positions in the original elbow sequence of the heavy chains ( . . . VSSASTKG . . . ) to allow for the sequence topology required to achieve the light chain insert format. In some embodiments a small number of residues may be removed from the cut site in the elbow region.

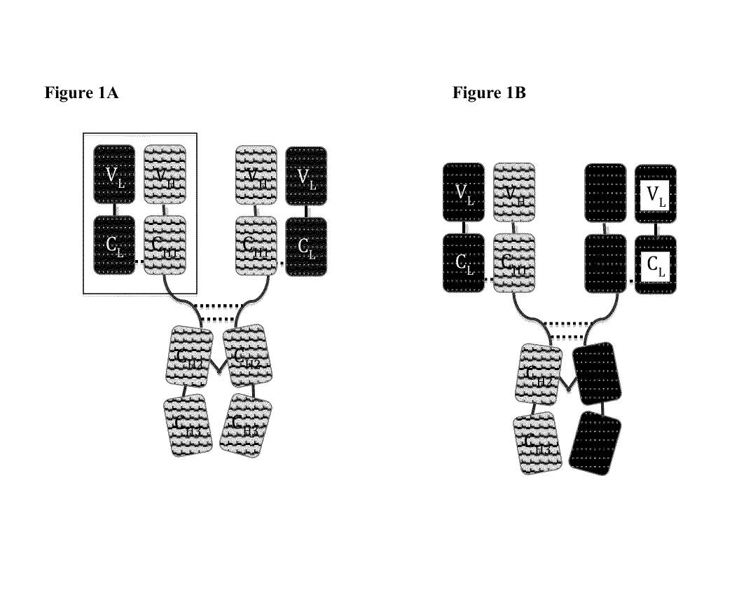

### DETAILED DESCRIPTION OF THE INVENTION

Previous attempts to produce a bispecific antibody include strategies such as fusing two hybridoma cell lines expressing monospecific, bivalent antibodies with the respective specificities (“quadroma technology”) (Milstein C, Cuello A C (1983) Hybrid hybridomas and their use in immunohistochemistry. Nature 305:537-540). However, it was immediately apparent that simultaneous expression of two different heavy chains and two different light chains according to the strategy described by Milstein leads to an almost inseparable mixture of 10 almost identical compounds containing only minor amounts of the desired bispecific antibody. (Suresh M R, Cuello A C, Milstein C (1986) Bispecific monoclonal antibodies from hybrid hybridomas. Methods Enzymol 121:210-228).

The important challenges in the design of selective bispecific antibody include effective induction of heterodimerization of the two heavy chains; and selective pairing of light-chain and heavy-chain. Strategies for the induction of selective heterodimerization of the heavy chains are provided for instance in WO/2012/058768 that describes antibody constructs comprising heavy chains that are asymmetric in the various domains (e.g. CH2 and CH3), wherein each heavy chain is modified to form the desired heterodimer with high selectivity and purity; and wherein the resultant heavy chain heterodimer has a stability comparable to the native homodimer.

The selective pairing of the light and heavy chain has been a difficult problem to address because a total of four possible pairings of heavy and light chains remain, only one of which represents the desired compound. Provided herein are methods of overcoming this problem, leading to the selective assembly of an asymmetric multispecific IgG-like antibody. In certain embodiments, provided herein are asymmetric bispecific antibody constructs comprising at least two different antigen binding single-chain Fab segments attached to the N-terminus of the Fc region, wherein each said single-chain Fab segment comprises selective pairing of the light and heavy chains, and wherein each said single-chain Fab segment recognizes a different antigen.

Provided is a method of engineering features in the Fab portion of the antibody so as to facilitate selective pairing of the obligate light and heavy chain domains. The successive expression of the obligate domains in a serial manner provides a kinetically favorable opportunity for the neighboring domains to interact and pair up preferentially. The loops between the VH and VL are of a length sufficient to allow the natural Fv like packing between the two domains. Similarly, the loop between the CL and CH1 domains are of an appropriate length so as to permit the natural interaction between these two domains. It has been established that CH1 is the slowest folding domain in the antibody structure and the folding of this domain is induced by the local presence of the CL domain. The folding of the CH1 domain is coupled to its pairing with the CL domain. The sequential expression of the CL and CH1 domain sequences in the light chain insert design presented here facilitates the CH1 domain folding and pairing with its obligate CL domain

Using the conventional approach involving coexpression of the light and heavy chains of the antibody in order to form a bispecific molecule, there is a need to express two different heavy and two different light chains. This leads to the formation of incorrect heavy and light chain pairs apart from the bispecific product of interest and requires complex purification in order to separate the correctly paired bispecific species of interest. The immunoglobulin constructs described herein comprise single chain Fab polypeptides and single chain Fvs which when combined with appropriate constant domain polypeptides allow the formation of correctly paired antibody structures.

Provided herein is a method of designing asymmetric, bispecific antibody molecules comprising at least two different single-chain Fab segments, wherein each said single-chain Fab segment is connected to a heavy chain polypeptide.

In certain embodiments, are provided methods of designing bispecific antibodies comprising single-chain Fab segments based on two independently developed monospecific antibody molecules. Provided herein is a method of designing antibody constructs, wherein said method comprises pairing up two different heavy chains, each selectively paired to its obligate light chain.

Unless defined otherwise, all technical and scientific terms used herein have the same meaning as is commonly understood by one of skill in the art to which the claimed subject matter belongs. In the event that there are a plurality of definitions for terms herein, those in this section prevail. Where reference is made to a URL or other such identifier or address, it is understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise.

Amino acid modifications utilized to generate a modified CH3 domain include, but are not limited to, amino acid insertions, deletions, substitutions, and rearrangements. The modifications of the CH3 domain and the modified CH3 domains are referred to herein collectively as “CH3 modifications”, “modified CH3 domains”, “variant CH3 domains” or “CH3 variants”. In certain embodiments, the hese modified CH3 domains are incorporated into a molecule of choice. Accordingly, in one embodiment are provided molecules, for instance polypeptides, such as immunoglobulins (e.g., antibodies) and other binding proteins, comprising an Fc region (as used herein “Fc region” and similar terms encompass any heavy chain constant region domain comprising at least a portion of the CH3 domain) incorporating a modified CH3 domain. Molecules comprising Fc regions comprising a modified CH3 domain (e.g., a CH3 domain comprising one or more amino acid insertions, deletions, substitutions, or rearrangements) are referred to herein as “Fc variants”, “heterodimers” or “heteromultimers”. The present Fc variants comprise a CH3 domain that has been asymmetrically modified to generate heterodimer Fc variants or regions. The Fc region is comprised of two heavy chain constant domain polypetides—Chain A and Chain B, which can be used interchangeably provided that each Fc region comprises one Chain A and one Chain B polypeptide. The amino acid modifications are introduced into the CH3 in an asymmetric fashion resulting in a heterodimer when two modified CH3 domains form an Fc variant (See, e.g., Table 1). As used herein, asymmetric amino acid modifications are any modification wherein an amino acid at a specific position on one polypeptide (e.g., “Chain A”) is different from the amino acid on the second polypeptide (e.g., “Chain B”) at the same position of the heterodimer or Fc variant. This can be a result of modification of only one of the two amino acids or modification of both amino acids to two different amino acids from Chain A and Chain B of the Fc variant. It is understood that the variant CH3 domains comprise one or more asymmetric amino acid modifications.

As used herein, “isolated” construct means a construct that has been identified and separated and/or recovered from a component of its natural cell culture environment. Contaminant components of its natural environment are materials that would interfere with diagnostic or therapeutic uses for the immunoglobulin construct, and may include enzymes, hormones, and other proteinaceous or non-proteinaceous solutes.

The variant Fc heterodimers are generally purified to substantial homogeneity. The phrases “substantially homogeneous”, “substantially homogeneous form” and “substantial homogeneity” are used to indicate that the product is substantially devoid of by-products originated from undesired polypeptide combinations (e.g. homodimers). Expressed in terms of purity, substantial homogeneity means that the amount of by-products does not exceed 10%, and preferably is below 5%, more preferably below 1%, most preferably below 0.5%, wherein the percentages are by weight.

Terms understood by those in the art of antibody technology are each given the meaning acquired in the art, unless expressly defined differently herein. Antibodies are known to have variable regions, a hinge region, and constant domains. Immunoglobulin structure and function are reviewed, for example, in Harlow et al, Eds., Antibodies: A Laboratory Manual, Chapter 14 (Cold Spring Harbor Laboratory, Cold Spring Harbor, 1988).

In the present description, any concentration range, percentage range, ratio range, or integer range is to be understood to include the value of any integer within the recited range and, when appropriate, fractions thereof (such as one tenth and one hundredth of an integer), unless otherwise indicated. As used herein, “about” means±10% of the indicated range, value, sequence, or structure, unless otherwise indicated. It should be understood that the terms “a” and “an” as used herein refer to “one or more” of the enumerated components unless otherwise indicated or dictated by its context. The use of the alternative (e.g., “or”) should be understood to mean either one, both, or any combination thereof of the alternatives. As used herein, the terms “include” and “comprise” are used synonymously. In addition, it should be understood that the individual single chain polypeptides or immunoglobulin constructs derived from various combinations of the structures and substituents described herein are disclosed by the present application to the same extent as if each single chain polypeptide or heterodimer were set forth individually. Thus, selection of particular components to form individual single chain polypeptides or heterodimers is within the scope of the present disclosure.

The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety for any purpose.

It is to be understood that the methods and compositions described herein are not limited to the particular methodology, protocols, cell lines, constructs, and reagents described herein and as such may vary. It is also to be understood that the terminology used herein is for the purpose of describing particular embodiments only, and is not intended to limit the scope of the methods and compositions described herein, which will be limited only by the appended claims.

All publications and patents mentioned herein are incorporated herein by reference in their entirety for the purpose of describing and disclosing, for example, the constructs and methodologies that are described in the publications, which might be used in connection with the methods, compositions and compounds described herein. The publications discussed herein are provided solely for their disclosure prior to the filing date of the present application. Nothing herein is to be construed as an admission that the inventors described herein are not entitled to antedate such disclosure by virtue of prior invention or for any other reason.

In the present application, amino acid names and atom names (e.g. N, O, C, etc.) are used as defined by the Protein DataBank (PDB) (www.pdb.org), which is based on the IUPAC nomenclature (IUPAC Nomenclature and Symbolism for Amino Acids and Peptides (residue names, atom names etc.), Eur. J. Biochem., 138, 9-37 (1984) together with their corrections in Eur. J. Biochem., 152, 1 (1985). The term “amino acid residue” is primarily intended to indicate an amino acid residue contained in the group consisting of the 20 naturally occurring amino acids, i.e. alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues.

The terms “polypeptide,” “peptide” and “protein” are used interchangeably herein to refer to a polymer of amino acid residues. That is, a description directed to a polypeptide applies equally to a description of a peptide and a description of a protein, and vice versa. The terms apply to naturally occurring amino acid polymers as well as amino acid polymers in which one or more amino acid residues is a non-naturally encoded amino acid. As used herein, the terms encompass amino acid chains of any length, including full length proteins, wherein the amino acid residues are linked by covalent peptide bonds.

The term “nucleotide sequence” is intended to indicate a consecutive stretch of two or more nucleotide molecules. The nucleotide sequence may be of genomic, cDNA, RNA, semisynthetic or synthetic origin, or any combination thereof.

The term “polymerase chain reaction” or “PCR” generally refers to a method for amplification of a desired nucleotide sequence in vitro, as described, for example, in U.S. Pat. No. 4,683,195. In general, the PCR method involves repeated cycles of primer extension synthesis, using oligonucleotide primers capable of hybridising preferentially to a template nucleic acid.

“Cell”, “host cell”, “cell line” and “cell culture” are used interchangeably herein and all such terms should be understood to include progeny resulting from growth or culturing of a cell. “Transformation” and “transfection” are used interchangeably to refer to the process of introducing DNA into a cell.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, praline, serine, threonine, tryptophan, tyrosine, and valine) and pyrrolysine and selenocysteine. Amino acid analogs refers to compounds that have the same basic chemical structure as a naturally occurring amino acid, i.e., an a carbon that is bound to a hydrogen, a carboxyl group, an amino group, and an R group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid. Reference to an amino acid includes, for example, naturally occurring proteogenic L-amino acids; D-amino acids, chemically modified amino acids such as amino acid variants and derivatives; naturally occurring non-proteogenic amino acids such as □-alanine, ornithine, etc.; and chemically synthesized compounds having properties known in the art to be characteristic of amino acids. Examples of non-naturally occurring amino acids include, but are not limited to, α-methyl amino acids (e.g. α-methyl alanine), D-amino acids, histidine-like amino acids (e.g., 2-amino-histidine, β-hydroxy-histidine, homohistidine), amino acids having an extra methylene in the side chain (“homo” amino acids), and amino acids in which a carboxylic acid functional group in the side chain is replaced with a sulfonic acid group (e.g., cysteic acid). The incorporation of non-natural amino acids, including synthetic non-native amino acids, substituted amino acids, or one or more D-amino acids into the proteins of the present invention may be advantageous in a number of different ways. D-amino acid-containing peptides, etc., exhibit increased stability in vitro or in vivo compared to L-amino acid-containing counterparts. Thus, the construction of peptides, etc., incorporating D-amino acids can be particularly useful when greater intracellular stability is desired or required. More specifically, D-peptides, etc., are resistant to endogenous peptidases and proteases, thereby providing improved bioavailability of the molecule, and prolonged lifetimes in vivo when such properties are desirable. Additionally, D-peptides, etc., cannot be processed efficiently for major histocompatibility complex class II-restricted presentation to T helper cells, and are therefore, less likely to induce humoral immune responses in the whole organism.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, “conservatively modified variants” refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of ordinary skill in the art will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of ordinary skill in the art will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the deletion of an amino acid, addition of an amino acid, or substitution of an amino acid with a chemically similar amino acid. Conservative substitution tables providing functionally similar amino acids are known to those of ordinary skill in the art. Such conservatively modified variants are in addition to and do not exclude polymorphic variants, interspecies homologs, and alleles of the invention.

Conservative substitution tables providing functionally similar amino acids are known to those of ordinary skill in the art. The following eight groups each contain amino acids that are conservative substitutions for one another:

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and [0139] 8) Cysteine (C), Methionine (M)

(see, e.g., Creighton, Proteins: Structures and Molecular Properties (W H Freeman & Co.; 2nd edition (December 1993).

The terms “identical” or percent “identity,” in the context of two or more nucleic acids or polypeptide sequences, refer to two or more sequences or subsequences that are the same. Sequences are “substantially identical” if they have a percentage of amino acid residues or nucleotides that are the same (i.e., about 50% identity, about 55% identity, 60% identity, about 65%, about 70%, about 75%, about 80%, about 85%, about 90%, or about 95% identity over a specified region), when compared and aligned for maximum correspondence over a comparison window, or designated region as measured using one of the following sequence comparison algorithms (or other algorithms available to persons of ordinary skill in the art) or by manual alignment and visual inspection. This definition also refers to the complement of a test sequence. The identity can exist over a region that is at least about 50 amino acids or nucleotides in length, or over a region that is 75-100 amino acids or nucleotides in length, or, where not specified, across the entire sequence of a polynucleotide or polypeptide. A polynucleotide encoding a polypeptide of the present invention, including homologs from species other than human, may be obtained by a process comprising the steps of screening a library under stringent hybridization conditions with a labeled probe having a polynucleotide sequence of the invention or a fragment thereof, and isolating full-length cDNA and genomic clones containing said polynucleotide sequence. Such hybridization techniques are well known to the skilled artisan.

A derivative, or a variant of a polypeptide is said to share “homology” or be “homologous” with the peptide if the amino acid sequences of the derivative or variant has at least 50% identity with a 100 amino acid sequence from the original peptide. In certain embodiments, the derivative or variant is at least 75% the same as that of either the peptide or a fragment of the peptide having the same number of amino acid residues as the derivative. In certain embodiments, the derivative or variant is at least 85% the same as that of either the peptide or a fragment of the peptide having the same number of amino acid residues as the derivative. In certain embodiments, the amino acid sequence of the derivative is at least 90% the same as the peptide or a fragment of the peptide having the same number of amino acid residues as the derivative. In some embodiments, the amino acid sequence of the derivative is at least 95% the same as the peptide or a fragment of the peptide having the same number of amino acid residues as the derivative. In certain embodiments, the derivative or variant is at least 99% the same as that of either the peptide or a fragment of the peptide having the same number of amino acid residues as the derivative.

As used herein, “isolated” polypeptide or immunoglobulin construct means a construct or polypeptide that has been identified and separated and/or recovered from a component of its natural cell culture environment. Contaminant components of its natural environment are materials that would interfere with diagnostic or therapeutic uses for the immunoglobulin construct, and may include enzymes, hormones, and other proteinaceous or non-proteinaceous solutes.

The phrase “selectively (or specifically) hybridizes to” refers to the binding, duplexing, or hybridizing of a molecule only to a particular nucleotide sequence under stringent hybridization conditions when that sequence is present in a complex mixture (including but not limited to, total cellular or library DNA or RNA).

The phrase “stringent hybridization conditions” refers to hybridization of sequences of DNA, RNA, or other nucleic acids, or combinations thereof under conditions of low ionic strength and high temperature as is known in the art. Typically, under stringent conditions a probe will hybridize to its target subsequence in a complex mixture of nucleic acid (including but not limited to, total cellular or library DNA or RNA) but does not hybridize to other sequences in the complex mixture. Stringent conditions are sequence-dependent and will be different in different circumstances. Longer sequences hybridize specifically at higher temperatures. An extensive guide to the hybridization of nucleic acids is found in Tijssen, Laboratory Techniques in Biochemistry and Molecular Biology—Hybridization with Nucleic Probes, “Overview of principles of hybridization and the strategy of nucleic acid assays” (1993).

As used herein, an “antibody” refers to a polypeptide substantially encoded by an immunoglobulin gene or immunoglobulin genes, or fragments thereof, which specifically bind and recognize an analyte (antigen). The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively.

An exemplary immunoglobulin (antibody) structural unit is composed of two pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively. In certain embodiments, the immunoglobulin constructs comprise at least one immunoglobulin domain from IgG, IgM, IgA, IgD, or IgE connected to a therapeutic polypeptide. In some embodiments, the immunoglobulin domain comprised in an immunoglobulin construct provided herein, is from an immunoglobulin based construct such as a diabody, or a nanobody. In certain embodiments, the immunoglobulin constructs described herein comprise at least one immunoglobulin domain from a heavy chain antibody such as a camelid antibody. In certain embodiments, the immunoglobulin constructs provided herein comprise at least one immunoglobulin domain from a mammalian antibody such as a bovine antibody, a human antibody, a camelid antibody, a mouse antibody or any chimeric antibody.

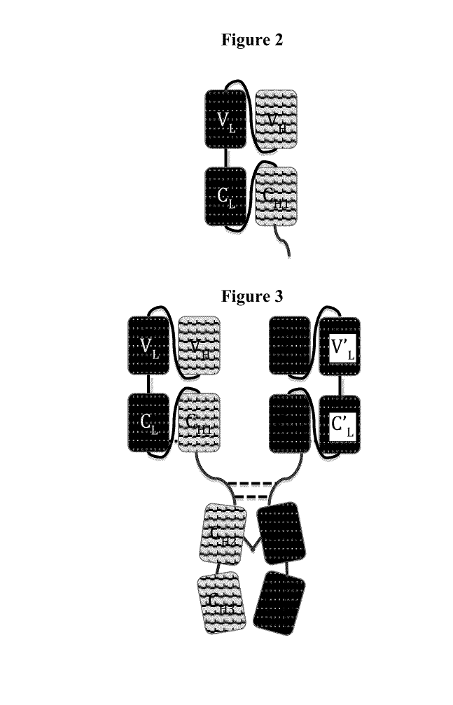

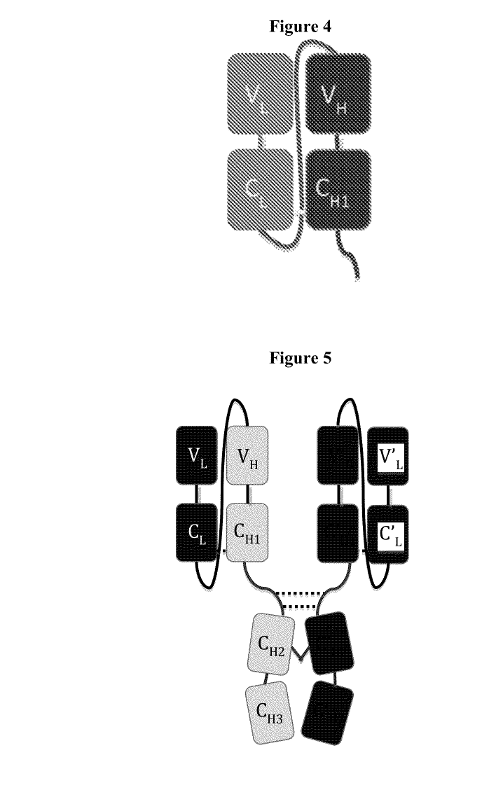

A “single-chain Fab segment” or (“single-chain Fab) (see for instance FIG. 2, FIG. 4) is a polypeptide comprising of an antibody heavy chain variable domain (VH), an antibody constant domain 1 (CH1), an antibody light chain variable domain (VL), an antibody light chain constant domain (CL) and a linker. In certain embodiments of the single-chain Fab segments described herein, the antibody domains and the linker have a sequence from the N-terminal to C-terminal comprising: VH-linker-VL-CL-linker-CH1. In certain embodiments of the single-chain Fab segments described herein, the antibody domains and the linker have a sequence from the N-terminal to C-terminal comprising: VL-CL-linker-VH-CH1. In certain embodiments of the single-chain Fab segments, each linker is a polypeptide. In some embodiments, each linker is a polypeptide comprising from about 3 to about 100 amino acids. In certain embodiments of the single-chain Fab segments, each linker is a polypeptide comprising from about 5 to about 50 amino acids. In some embodiments of the single-chain Fab segments, each linker is a polypeptide comprising at least 10 amino acids. In some embodiments of the single-chain Fab segments, each linker is a polypeptide comprising at least 20 amino acids. In certain embodiments of the single-chain Fab segments, at least one of the linkers is a polypeptide of at least 30 amino acids. In certain embodiments of the single-chain Fab segments, at least one linker is a polypeptide of about 30 to about 50 amino acids. In certain embodiments of the single-chain Fab segments, at least one linker is a polypeptide of about 35 to about 50 amino acids. In some embodiments of the single-chain Fab segments, each linker is a polypeptide of about 30 to about 50 amino acids. In certain embodiments, each linker is a polypeptide of about 35 to about 50 amino acids. In certain embodiments of the single-chain Fab segments, each linker is a polypeptide of about 30 amino acids or lesser. In certain embodiments, the single-chain Fab segments comprise at least one linker which is a polypeptide of about 29 amino acids or lesser. In certain embodiments, the single-chain Fab segments comprise at least one linker which is a polypeptide of about 3 amino acids to about 29 amino acids. In certain embodiments, the single-chain Fab segments comprise at least one linker which is a polypeptide of about 32 amino acids or lesser. In certain embodiments, the single-chain Fab segments comprise at least one stabilizing disulfide bond between a light chain domain and a heavy chain domain.

As used herein, the ‘light Chain insert design’ or ‘light chain insert strategy’ or “light chain insert’ (LCI) as described herein refers to the design of immunoglobulin constructs comprising single chain Fv polypeptides connected to heavy chain polypeptides as shown in FIG. 3. In certain embodiments, ‘light chain Insert design’ or ‘light chain insert strategy’ or ‘light chain insert’ refers to the design of a single chain Fab by connecting a single chain Fv polypeptide to a constant domain polypeptide as shown in FIG. 2. In some embodiments is a single chain Fab region (scFab) that comprises a variable region polypeptide (VH) from an immunoglobulin heavy chain, a variable region polypeptide (VL) from an immunoglobulin light chain, a constant region polypeptide (CL) from an immunoglobulin light chain, and a constant region polypeptide (CH1) from an immunoglobulin heavy chain; wherein VH and VL polypeptides are connected by a first linker to form a single chain Fv construct (scFv). In some embodiments, said CL and CH1 are connected by a second linker. In certain embodiments, the immunoglobulin construct has a sequence comprising VH-L1-VL-CL-L2-CH1, wherein L1 and L2 are first and second linkers. In certain embodiments, the immunoglobulin construct has a sequence comprising VH-L1-VL-L3-CL-L2-CH1, wherein L1, L2 and L3 are linkers. In an embodiment, the immunoglobulin construct has a sequence comprising VL-L4-VH-CH1-L5-CL, wherein L4 and L5 are linkers.

In certain embodiments, each linker is a polypeptide comprising from about 1 to about 100 amino acids. In specific embodiments, each linker is a polypeptide comprising from about 1 to about 50 amino acids. In specific embodiments, each linker is a polypeptide comprising from about 10 to about 25 amino acids. In some embodiments, the linker comprises an amino acid sequence comprising amino acids selected from Gly (G), Ser (S) and Glu (E). In an embodiment, said linker is comprised of polypeptide of the general formula (Gly-Gly-Gly-Ser)n wherein n is an integer from 4 to 10. In certain embodiments, at least one linker is selected based on its ability to increase ease of purification of the construct.

As used herein, the ‘long linker design’ or ‘long linker strategy’ or ‘long linker’ (LL) as described herein refers to the design of multispecific or bispecific immunoglobulin constructs comprising single chain Fab polypeptides connected to heavy chain polypeptides as shown in FIG. 5. In certain embodiments, ‘long linker design’ or ‘long linker strategy’ or ‘long linker’ refers to the design of a single chain Fab as shown in FIG. 4. In certain embodiments, the long linker comprises a linker polypeptide that has a propensity to form a helix. In some embodiments, at least about 25% of the linker exists in helical form. In some embodiments, at least about 50% of the linker exists in helical form. In certain other embodiments, at least about 60% of the linker exists in helical form. In another embodiment, at least about 75% of the linker exists in helical form. In another embodiment, at least about 80% of the linker exists in helical form. In further embodiments, at least about 90% of the linker exists in helical form. In further embodiments, at least about 95% of the linker exists in helical form. In certain embodiment, the linker comprises multiple helical segments. In certain embodiments, the helical linker is selected based on its ability to increase ease of purification of the construct.

Provided are immunoglobulin constructs described herein wherein the linker polypeptide forms at least one of an alpha helix, a polyproline type I helix, a polyproline type II helix and a 3₁₀ helix. In some embodiments, the linker forms between about 1 turn to about 20 turns of a helix. In an embodiment, the linker forms between about 3 turn to about 5 turns of a helix. In an embodiment, the linker forms between about 2 turn to about 4 turns of a helix. In an embodiment, the linker forms between about 2 turn to about 10 turns of a helix. In some embodiments, the linker comprises at least one pair of amino acids that form helix stabilizing interactions. In an embodiment, the helix stabilizing interaction is at least one of a charge-charge interaction, a cation-pi interaction, a hydrophobic interaction and a size complimentary interaction.

Provided are isolated immunoglobulin constructs described herein, wherein said construct comprises at least one linker polypeptide with propensity to form a helix, and wherein said linker polypeptide comprises amino acids selected from Gly (G), Ser (S), Glu (E), Gln (Q), Asp (D), Asn (N), Arg (R), Lys (K), His (H), Val (V) and Ile (I). In certain embodiments, the linker polypeptide comprises amino acids selected from Met (M), Ala (A), Leu (L), Glu (E) and Lys (K). In an embodiment, the linker polypeptide comprises at least one Pro (P) residue. In certain embodiments, the linker has an amino acid sequence comprising at least one (Asp-Asp-Ala-Lys-Lys)n motif wherein n is an integer from 1 to 10.

Provided herein are immunoglobulin constructs comprising: a first polypeptide construct comprising a first scFab described herein; and a first heavy chain polypeptide comprising a first CH3 region; and a second polypeptide construct comprising a second heavy chain polypeptide comprising a second CH3 region, wherein at least one of said first and second heavy chain polypeptides optionally comprises a variant CH3 region that promotes the formation of a heterodimer. In some embodiments, said first and second polypeptide construct further comprising an antigen binding polypeptide construct. In an embodiment, the antigen binding polypeptide construct is at least one of an scFv or a scFab. In some embodiments, the scFab is an scFab described herein.

In certain embodiments, depending on the required variant in the form of a scFab or a scMab, other immunoglobulin constant domains are included in the polypeptide sequence and the nucleic acid sequence encoding said polypeptide. In certain embodiments, scMab constructs comprise of VH, VL, CL, CH1, CH2 and CH3 domains with linkers as described herein and natural hinge regions present. In some embodiments, wild type CH3 domain sequences are employed to achieve bivalent monospecific antibody molecules. In some embodiments, mutated versions of the CH2 and CH3 domains are employed that alter FcRn binding or favor CH3 heterodimer formation. In some embodiments the CH2 sequence is not included in the polypeptide sequence of interest. In some embodiments, the CH3 domain comprises mutations that result in heterodimeric Fc formation. In some embodiments, heterodimeric Fc forming sequences are employed to achieve bivalent bispecific antibody molecules.

In some embodiments, the first and second heavy chain polypeptides form a heterodimeric Fc. In certain embodiments, the heterodimeric Fc comprises a variant immunoglobulin CH3 domain comprising at least one amino acid mutation. In certain embodiments, said at least one amino acid mutation promotes the formation of said heterodimeric Fc with stability comparable to a native homodimeric Fc. In an embodiment, the variant CH3 domain has a melting temperature (Tm) of about 73° C. or greater. In an embodiment, the heterodimeric Fc is formed with a purity of at least about 90%. In some embodiments, the heterodimeric Fc is formed with a purity of at least about 98% and the Tm is at least about 73° C. In another embodiment, the heterodimeric Fc is formed with a purity of at least about 90% and the Tm is about 75° C.

Provided is an immunoglobulin construct described herein, wherein at least one of said first and second heavy chain polypeptides further comprises a variant CH2 domain comprising amino acid modifications to promote selective binding to at least one of the Fcgamma receptors. In an embodiment, at least one of said first and second heavy chain polypeptides comprises a variant CH2 domain or hinge comprising amino acid modifications that prevents functionally effective binding to at least one of the Fcgamma receptors. In some embodiments, the Fc region is glycosylated. In some embodiments, the Fc region is aglycosylated. In an embodiment, the Fc region is fucosylated. In another embodiment, the Fc region is afucosylated.

In some embodiments is provided an immunoglobulin construct described herein, wherein said immunoglobulin construct is a multispecific immunoglobulin construct. In an embodiment, wherein the immunoglobulin construct is bispecific.

Provided is an isolated immunoglobulin construct comprising: a first monomeric polypeptide comprising a first single chain Fv polypeptide connected by a linker to a first constant domain polypeptide; and a second monomeric polypeptide comprising a second single chain Fv polypeptide which is different from said first single chain Fv polypeptide, connected by a linker to a second constant domain polypeptide which is different from said first constant domain polypeptide; each said constant domain polypeptide comprising at least one each of a CL region, a CH1 region, and a CH3 region or fragments, variants or derivatives thereof; and wherein said CL and CH1 regions are connected by a linker, and wherein said first and second constant domain polypeptides form a Fc region. In some embodiments, the construct does not contain any CH2 domains.

In an embodiment is provided an isolated immunoglobulin construct comprising: a first monomeric polypeptide comprising a first scFab polypeptide fused to a first constant domain polypeptide; and a second monomeric polypeptide comprising a second scFab polypeptide which is different from said first Fab polypeptide, fused to a second constant domain polypeptide; wherein at least one of said first and second scFab polypeptides comprises a linker polypeptide with a propensity to form a helical structure; and wherein said first and second constant domain polypeptides form a heterodimeric Fc region comprising a variant immunoglobulin CH3 region comprising at least one amino acid mutation that promotes the formation of said heterodimer with stability comparable to a native homodimeric Fc.

Provided herein is an isolated immunoglobulin construct comprising a first monomeric polypeptide comprising a first single chain Fv polypeptide connected to a first constant domain polypeptide; and a second monomeric polypeptide comprising a second single chain Fv polypeptide connected to a second constant domain polypeptide; each said constant domain polypeptide comprising at least one each of a CL domain, a CH1 domain, a CH2 domain and a CH3 domain or fragments, variants or derivatives thereof; wherein said first and second constant domain polypeptides form a Fc region.

In certain embodiments is an isolated immunoglobulin construct described herein, wherein at least one single chain Fv polypeptide is connected by the linker to the CL domain of the corresponding constant domain polypeptide. Provided in certain embodiments is an isolated immunoglobulin construct described herein, wherein at least one single chain Fv polypeptide is connected by the linker to the CH1 domain of the corresponding constant domain polypeptide. In certain embodiments is provided an immunoglobulin construct described herein, wherein at least one monomeric polypeptide has a sequence comprising VH-L1-VL-CL-L2-CH1-CH2-CH3, wherein L1 and L2 are linkers.

In certain embodiments is an isolated immunoglobulin construct described herein, wherein at least one monomeric polypeptide has a sequence comprising VH-L3-VL-L4-CH1-L5-CL-CH2-CH3, wherein L3, L4 and L5 are linkers. In certain embodiments is an isolated immunoglobulin construct described herein, wherein at least one monomeric polypeptide has a sequence comprising VL-L6-VH-CH1-L7-CL-CH2-CH3, wherein L6 and L7 are linkers. In certain embodiments is an isolated immunoglobulin construct described herein, said constant domain polypeptides optionally comprising at least one linker connecting one or more of said CL domain, CH1 domain, CH2 domain and CH3 domain.

In certain embodiments is an isolated immunoglobulin construct described herein, wherein said first and second constant domain polypeptide form a heterodimeric Fc region. In certain embodiments the heterodimeric Fc region comprises a variant immunoglobulin CH3 domain comprising at least one amino acid mutation. In some embodiments, the at least one amino acid mutation promotes the formation of said heterodimeric Fc region with stability comparable to a native homodimeric Fc. In some embodiments, the variant CH3 domain has a melting temperature (Tm) of about 73° C. or greater. In certain embodiments, the heterodimer Fc region is formed with a purity greater than about 90%. In certain embodiments, the heterodimer Fc region is formed with a purity of at least about 98% or greater and the Tm is at least about 73° C. In some embodiments, the heterodimer Fc region is formed with a purity of about 90% or greater and the Tm is about 75° C.

In certain embodiments is an isolated immunoglobulin construct described herein, comprising at least one constant domain polypeptide comprising a variant CH2 domain comprising amino acid modifications to promote selective binding to at least one of the Fcgamma receptors. In some embodiments, at least one constant domain polypeptide comprises a variant CH2 domain or hinge comprising amino acid modifications that prevents functionally effective binding to at least one of the Fcgamma receptors.

In certain embodiments is an isolated immunoglobulin construct described herein, wherein the Fc region is glycosylated. In some embodiments is an isolated immunoglobulin construct described herein, wherein the Fc region is aglycosylated.

In certain embodiments is an isolated immunoglobulin construct described herein wherein each linker is a polypeptide comprising from about 1 to about 100 amino acids. In some embodiments, linker polypeptides have the general formula (Gly-Gly-Gly-Ser)n wherein n is an integer from 1 to 20.

Provided is an isolated immunoglobulin construct described herein, wherein said immunoglobulin construct is a multispecific immunoglobulin construct. In some embodiments, the immunoglobulin construct is bispecific.

Provided is an isolated bispecific immunoglobulin construct comprising a first monomeric polypeptide comprising a first single chain Fv polypeptide connected by a linker to a first constant domain polypeptide; and a second monomeric polypeptide comprising a second single chain Fv polypeptide which is different from said first Fv polypeptide, connected by a linker to a second constant domain polypeptide which is different from said first constant domain polypeptide; each said constant domain polypeptide comprising at least one each of a CL domain, a CH1 domain, a CH2 region and a CH3 region or fragments, variants or derivatives thereof; and wherein said first and second constant domain polypeptides form a Fc region.

Provided herein is an isolated immunoglobulin construct comprising a first monomeric polypeptide comprising a first single chain Fv polypeptide connected to a first constant domain polypeptide; and a second monomeric polypeptide comprising a second single chain Fv polypeptide, connected to a second constant domain polypeptide; each said constant domain polypeptide comprising at least one each of a CL domain, a CH1 domain, a CH2 domain and a CH3 domain or fragments, variants or derivatives thereof; and wherein said first and second constant domain polypeptides form a Fc region.

Provided herein is an isolated bispecific immunoglobulin construct comprising a first monomeric polypeptide comprising a first single chain Fv polypeptide connected by a linker to a first constant domain polypeptide; and a second monomeric polypeptide comprising a second single chain Fv polypeptide which is different from said first Fv polypeptide, connected by a linker to a second constant domain polypeptide which is different from said first constant domain polypeptide; each said constant domain polypeptide comprising at least one each of a CL domain, a CH1 domain, and a CH3 region or fragments, variants or derivatives thereof; and wherein said first and second constant domain polypeptides form a Fc region. Provided herein is an isolated bispecific immunoglobulin construct comprising a first monomeric polypeptide comprising a first single chain Fv polypeptide connected to a first constant domain polypeptide; and a second monomeric polypeptide comprising a second single chain Fv polypeptide, connected to a second constant domain polypeptide; each said constant domain polypeptide comprising at least one each of a CL domain, a CH1 domain and a CH3 domain or fragments, variants or derivatives thereof; and wherein said first and second constant domain polypeptides form a Fc region. In certain embodiments, the construct does not contain any CH2 domains.

Provided herein is a single chain Fab polypeptide comprising a single chain Fv polypeptide connected to a constant domain polypeptide, said constant domain polypeptide comprising at least a CL domain and a CH1 domain.

Provided herein is an immunoglobulin construct comprising a first monomeric polypeptide comprising a first single chain Fab polypeptide fused to a first constant domain polypeptide; and a second monomeric polypeptide comprising a second single chain Fab polypeptide which is different from said first Fab polypeptide, fused to a second constant domain polypeptide; wherein said first and second constant domain polypeptides form a heterodimeric Fc region comprising a variant immunoglobulin CH3 region comprising at least one amino acid mutation that promotes the formation of said heterodimeric Fc with stability comparable to a native homodimeric Fc. In certain embodiments is an isolated multispecific immunoglobulin construct described herein, wherein at least one of said first single chain Fab polypeptide and said second single chain Fab polypeptide has a sequence comprising VH-L8-VL-CL-L9-CH1; wherein L8 and L9 are linkers. In certain embodiments is an isolated multispecific immunoglobulin construct described herein, wherein at least one of said first single chain Fab polypeptide and said second single chain Fab polypeptide has a sequence comprising VL-CL-L10-VH-CH1; wherein L10 is a linker.

Provided herein is a pharmaceutical composition comprising an isolated immunoglobulin construct as defined herein; and a suitable excipient. Also provided is a process for the production of such a pharmaceutical composition, said process comprising: culturing a host cell under conditions allowing the expression of an immunoglobulin construct as defined herein; recovering the produced immunoglobulin construct from the culture; and producing the pharmaceutical composition.

In certain embodiments is provided a method of treating cancer in a mammal in need thereof, comprising administering to the mammal a composition comprising an effective amount of the pharmaceutical composition described herein. In certain embodiments, the cancer is a solid tumor. In some embodiments, the solid tumor is one or more of sarcoma, carcinoma, and lymphoma. In some embodiments, the cancer is one or more of B-cell lymphoma, non-Hodgkin's lymphoma, and leukemia.

In certain embodiments is provided a method of treating an autoimmune condition in a mammal in need thereof, comprising administering to said mammal a composition comprising an effective amount of the pharmaceutical composition described herein. In certain embodiments, the autoimmune condition is one or more of multiple sclerosis, rheumatoid arthritis, lupus erytematosus, psoriatic arthritis, psoriasis, vasculitis, uveitis, Crohn's disease, and type 1 diabetes.

In certain embodiments is provided a method of treating an inflammatory condition in a mammal in need thereof, comprising administering to said mammal a composition comprising an effective amount of the pharmaceutical composition described herein.

Provided herein are host cells comprising nucleic acid encoding an immunoglobulin construct described herein. In certain embodiments, the nucleic acid encoding the first monomeric protein and the nucleic acid encoding the second monomeric protein are present in a single vector. In certain embodiments, the nucleic acid encoding the first monomeric protein and the nucleic acid encoding the second monomeric protein are present in separate vectors.

Also provided is a kit comprising an immunoglobulin construct as defined herein, and instructions for use thereof.

Functional Activity:

“A polypeptide having functional activity” refers to a polypeptide capable of displaying one or more known functional activities associated with a full-length/native protein. Such functional activities include, but are not limited to, biological activity, antigenicity [ability to bind (or compete with a polypeptide for binding) to an anti-polypeptide antibody], immunogenicity (ability to generate antibody which binds to a specific polypeptide described herein), ability to form multimers with polypeptides described herein, and ability to bind to a receptor or ligand for a polypeptide. In certain embodiments, the functional activity includes the ability to improve the expression and stability of a partner protein.

“A polypeptide having biological activity” refers to a polypeptide exhibiting activity similar to, but not necessarily identical to, an activity of a therapeutic protein described herein, including mature forms, as measured in a particular biological assay, with or without dose dependency. In the case where dose dependency does exist, it need not be identical to that of the polypeptide, but rather substantially similar to the dose-dependence in a given activity as compared to the polypeptide described herein (i.e., the candidate polypeptide will exhibit greater activity or not more than about 25-fold less, or not more than about tenfold less activity, or not more than about three-fold less activity relative to a polypeptide described herein, or presented in Table 2).

The immunoglobulin constructs described herein can be assayed for functional activity (e.g., biological activity) using or routinely modifying assays known in the art, as well as assays described herein.

In certain embodiments, where a binding partner (e.g., a receptor or a ligand) is identified for an immunoglobulin construct described herein, binding to that binding partner by an immunoglobulin construct described herein is assayed, e.g., by means well-known in the art, such as, for example, reducing and non-reducing gel chromatography, protein affinity chromatography, and affinity blotting. See generally, Phizicky et al., Microbiol. Rev. 59:94-123 (1995). In another embodiment, the ability of physiological correlates of an immunoglobulin construct to bind to a substrate(s) of polypeptides of the immunoglobulin construct can be routinely assayed using techniques known in the art.

Provided are immunoglobulin constructs described herein, wherein said construct binds at least one target antigen selected from CD3, CD19, HER2, Tissue factor and CD16a. In certain embodiments, are immunoglobulin constructs described herein that bind an antigen expressed by a cytotoxic cell. In certain embodiments, are immunoglobulin constructs described herein that bind an antigen expressed by a T cell. In some embodiments, the T cell is at least one of a T helper cell, a cytotoxic T cell and a natural killer T cell. In certain embodiments, are immunoglobulin constructs described herein that bind an antigen expressed by a cancer cell. In some embodiments, are immunoglobulin constructs described herein that bind an antigen expressed by an immune cell.

In an embodiment is provided an immunoglobulin construct described herein, wherein said construct can bind at least one T cell or Natural killer cell and at least one other cell that expresses an antigen. In an embodiment is provided an immunoglobulin construct described herein, wherein said construct can bind at least one T cell and at least one B cell. In some embodiments, the T cell is a human cell. In an embodiment, the T cell is a non-human, mammalian cell. In some embodiments, the immunoglobulin construct described herein binds an antigen expressed on a cell is associated with a disease. In some embodiments, the disease is a cancer. In an embodiment, the cancer is selected from a carcinoma, a sarcoma, leukaemia, lymphoma and glioma. In an embodiment, the cancer is at least one of a sarcoma, a blastoma, a papilloma and an adenoma. In some embodiments, the cancer is at least one of squamous cell carcinoma, adenocarcinoma, transition cell carcinoma, osteosarcoma and soft tissue sarcoma.

In some embodiments, the immunoglobulin construct described herein binds an antigen on at least one cell which is an autoimmune reactive cell. In some embodiments, the autoimmune reactive cell is a lymphoid or myeloid cell.

The term “effective amount” as used herein refers to that amount of immunoglobulin construct being administered, which will relieve to some extent one or more of the symptoms of the disease, condition or disorder being treated. Compositions containing the immunoglobulin construct described herein can be administered for prophylactic, enhancing, and/or therapeutic treatments.

Therapeutic Uses:

In an aspect, immunoglobulin constructs described herein are directed to antibody-based therapies which involve administering said construct, a fragment or variant thereof, to a patient for treating one or more of the disclosed diseases, disorders, or conditions. Therapeutic compounds described herein include, but are not limited to, immunoglobulin constructs described herein, nucleic acids encoding immunoglobulin constructs described herein.

In a specific embodiment, are antibody-based therapies which involve administering immunoglobulin constructs described herein comprising at least a fragment or variant of an antibody to a patient for treating one or more diseases, disorders, or conditions, including but not limited to: neural disorders, immune system disorders, muscular disorders, reproductive disorders, gastrointestinal disorders, pulmonary disorders, cardiovascular disorders, renal disorders, proliferative disorders, and/or cancerous diseases and conditions, and/or as described elsewhere herein.

A summary of the ways in which the immunoglobulin constructs are used therapeutically includes binding locally or systemically in the body or by direct cytotoxicity of the antibody, e.g. as mediated by complement (CDC) or by effector cells (ADCC). Some of these approaches are described in more detail below. Armed with the teachings provided herein, one of ordinary skill in the art will know how to use the immunoglobulin constructs described herein for diagnostic, monitoring or therapeutic purposes without undue experimentation.

The immunoglobulin constructs described herein, comprising at least a fragment or variant of an antibody may be administered alone or in combination with other types of treatments (e.g., radiation therapy, chemotherapy, hormonal therapy, immunotherapy and anti-tumor agents). Generally, administration of products of a species origin or species reactivity (in the case of antibodies) that is the same species as that of the patient is preferred. Thus, in an embodiment, human antibodies, fragments derivatives, analogs, or nucleic acids, are administered to a human patient for therapy or prophylaxis.

Gene Therapy:

In a specific embodiment, nucleic acids comprising sequences encoding immunoglobulin constructs described herein are administered to treat, inhibit or prevent a disease or disorder associated with aberrant expression and/or activity of a protein, by way of gene therapy. Gene therapy refers to therapy performed by the administration to a subject of an expressed or expressible nucleic acid. In this embodiment, the nucleic acids produce their encoded protein that mediates a therapeutic effect. Any of the methods for gene therapy available in the art can be used.

Demonstration of Therapeutic or Prophylactic Activity:

The immunoglobulin constructs or pharmaceutical compositions described herein are tested in vitro, and then in vivo for the desired therapeutic or prophylactic activity, prior to use in humans. For example, in vitro assays to demonstrate the therapeutic or prophylactic utility of a compound or pharmaceutical composition include, the effect of a compound on a cell line or a patient tissue sample. The effect of the compound or composition on the cell line and/or tissue sample can be determined utilizing techniques known to those of skill in the art including, but not limited to, rosette formation assays and cell lysis assays. In accordance with the invention, in vitro assays which can be used to determine whether administration of a specific compound is indicated, include in vitro cell culture assays in which a patient tissue sample is grown in culture, and exposed to or otherwise administered an immunoglobulin construct, and the effect of such immunoglobulin construct upon the tissue sample is observed.

Therapeutic/Prophylactic Administration and Composition

Provided are methods of treatment, inhibition and prophylaxis by administration to a subject of an effective amount of an immunoglobulin construct or pharmaceutical composition described herein. In an embodiment, the immunoglobulin construct is substantially purified (e.g., substantially free from substances that limit its effect or produce undesired side-effects). In certain embodiments, the subject is an animal, including but not limited to animals such as cows, pigs, horses, chickens, cats, dogs, etc., and in certain embodiments, a mammal, and most preferably human.

In an embodiment is a process for the production of a pharmaceutical composition described herein, said process comprising: culturing a host cell under conditions allowing the expression of an immunoglobulin construct as described herein; recovering the produced immunoglobulin construct from the culture; and producing the pharmaceutical composition.

Provided is a method of treating cancer in a mammal in need thereof, comprising administering to the mammal a composition comprising an effective amount of the pharmaceutical composition described herein. Also provided is a use of an immunoglobulin construct described herein in the treatment of cancer in a mammal in need thereof, comprising administering to the mammal a composition comprising an effective amount of the immunoglobulin construct described herein. In an embodiment the cancer is a solid tumor. In some embodiments, the solid tumor is one or more of sarcoma, carcinoma, and lymphoma. In an embodiment, the cancer is one or more of B-cell lymphoma, non-Hodgkin's lymphoma, and leukemia.

Provided is a method of treating an autoimmune condition in a mammal in need thereof, comprising administering to said mammal a composition comprising an effective amount of the pharmaceutical composition described herein. Also provided is a use of an immunoglobulin construct described herein in the treatment of an autoimmune disease, said use comprising providing a composition comprising an effective amount of the immunoglobulin construct described herein. In some embodiments, the autoimmune condition is one or more of multiple sclerosis, rheumatoid arthritis, lupus erytematosus, psoriatic arthritis, psoriasis, vasculitis, uveitis, Crohn's disease, and type 1 diabetes.

Provided is a method of treating an inflammatory condition in a mammal in need thereof, comprising administering to said mammal a composition comprising an effective amount of the pharmaceutical composition described herein. Also provided is use of an immunoglobulin construct in the treatment of an inflammatory condition in an individual, comprising providing to said individual an effective amount of an immunoglobulin construct described herein.

Various delivery systems are known and can be used to administer an immunoglobulin construct formulation described herein, e.g., encapsulation in liposomes, microparticles, microcapsules, recombinant cells capable of expressing the compound, receptor-mediated endocytosis (see, e.g., Wu and Wu, J. Biol. Chem. 262:4429-4432 (1987)), construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, in certain embodiments, it is desirable to introduce the immunoglobulin construct compositions described herein into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it is desirable to administer the immunoglobulin constructs, or compositions described herein locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody, of the invention, care must be taken to use materials to which the protein does not absorb.

In another embodiment, immunoglobulin constructs or composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the immunoglobulin constructs or composition can be delivered in a controlled release system. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, e.g., the brain, thus requiring only a fraction of the systemic dose (see, e.g., Goodson, in Medical Applications of Controlled Release, supra, vol. 2, pp. 115-138 (1984)).

Other controlled release systems are discussed in the review by Langer (Science 249:1527-1533 (1990)).

In a specific embodiment comprising a nucleic acid encoding an immunoglobulin construct described herein, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., Proc. Natl. Acad. Sci. USA 88:1864-1868 (1991)), etc. Alternatively, a nucleic acid can be introduced intracellularly and incorporated within host cell DNA for expression, by homologous recombination.

Also provided herein are pharmaceutical compositions. Such compositions comprise a therapeutically effective amount of an immunoglobulin construct, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

In certain embodiments, the composition comprising the immunoglobulin construct described herein is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In certain embodiments, the compositions described herein are formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxide isopropylamine, triethylamine, 2-ethylamino ethanol, histidine, procaine, etc.

The amount of the composition described herein which will be effective in the treatment, inhibition and prevention of a disease or disorder associated with aberrant expression and/or activity of a therapeutic protein can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses are extrapolated from dose-response curves derived from in vitro or animal model test systems.

Methods of Recombinant and Synthetic Production of Immunoglobulin Constructs:

Provided is a compositon comprising at least one expression vector for expressing an immunoglobulin construct described herein, comprising at least one nucleic acid sequence encoding said immunoglobulin construct.

In certain embodiments is a method of producing an expression product containing a an immunoglobulin construct described herein, in stable mammalian cells, the method comprising: transfecting at least one mammalian cell with: at least one DNA sequence encoding said immunoglobulin construct to generate stable mammalian cells; culturing said stable mammalian cells to produce said expression product comprising said immunoglobulin construct. In certain embodiments, the mammalian cell is selected from the group consisting of a VERO, HeLa, HEK, NS0, Chinese Hamster Ovary (CHO), W138, BHK, COS-7, Caco-2 and MDCK cell, and subclasses and variants thereof.

In certain embodiments are immunoglobulin constructs produced as recombinant molecules by secretion from yeast, a microorganism such as a bacterium, or a human or animal cell line. In embodiments, the polypeptides are secreted from the host cells.

Embodiments include a cell, such as a yeast cell transformed to express an immunoglobulin construct described herein. In addition to the transformed host cells themselves, are provided culture of those cells, preferably a monoclonal (clonally homogeneous) culture, or a culture derived from a monoclonal culture, in a nutrient medium. If the polypeptide is secreted, the medium will contain the polypeptide, with the cells, or without the cells if they have been filtered or centrifuged away. Many expression systems are known and may be used, including bacteria (for example E. coli and Bacillus subtilis), yeasts (for example Saccharomyces cerevisiae, Kluyveromyces lactis and Pichia pastoris, filamentous fungi (for example Aspergillus), plant cells, animal cells and insect cells.

An immunoglobulin construct described herein is produced in conventional ways, for example from a coding sequence inserted in the host chromosome or on a free plasmid. The yeasts are transformed with a coding sequence for the desired protein in any of the usual ways, for example electroporation. Methods for transformation of yeast by electroporation are disclosed in Becker & Guarente (1990) Methods Enzymol. 194, 182.

Successfully transformed cells, i.e., cells that contain a DNA construct of the present invention, can be identified by well known techniques. For example, cells resulting from the introduction of an expression construct can be grown to produce the desired polypeptide. Cells can be harvested and lysed and their DNA content examined for the presence of the DNA using a method such as that described by Southern (1975) J. Mol. Biol. 98, 503 or Berent et al. (1985) Biotech. 3, 208. Alternatively, the presence of the protein in the supernatant can be detected using antibodies.

Useful yeast plasmid vectors include pRS403-406 and pRS413-416 and are generally available from Stratagene Cloning Systems, La Jolla, Calif. 92037, USA. Plasmids pRS403, pRS404, pRS405 and pRS406 are Yeast Integrating plasmids (YIps) and incorporate the yeast selectable markers HIS3, 7RP1, LEU2 and URA3. Plasmids pRS413-416 are Yeast Centromere plasmids (Ycps).

A variety of methods have been developed to operably link DNA to vectors via complementary cohesive termini. For instance, complementary homopolymer tracts can be added to the DNA segment to be inserted to the vector DNA. The vector and DNA segment are then joined by hydrogen bonding between the complementary homopolymeric tails to form recombinant DNA molecules.

Synthetic linkers containing one or more restriction sites provide an alternative method of joining the DNA segment to vectors. The DNA segment, generated by endonuclease restriction digestion, is treated with bacteriophage T4 DNA polymerase or E. coli DNA polymerase 1, enzymes that remove protruding-single-stranded termini with their 3′ 5′-exonucleolytic activities, and fill in recessed 3′-ends with their polymerizing activities.

The combination of these activities therefore generates blunt-ended DNA segments. The blunt-ended segments are then incubated with a large molar excess of linker molecules in the presence of an enzyme that is able to catalyze the ligation of blunt-ended DNA molecules, such as bacteriophage T4 DNA ligase. Thus, the products of the reaction are DNA segments carrying polymeric linker sequences at their ends. These DNA segments are then cleaved with the appropriate restriction enzyme and ligated to an expression vector that has been cleaved with an enzyme that produces termini compatible with those of the DNA segment.

Synthetic linkers containing a variety of restriction endonuclease sites are commercially available from a number of sources including International Biotechnologies Inc, New Haven, Conn., USA.

Exemplary genera of yeast contemplated to be useful in the practice of the present invention as hosts for expressing the albumin, fusion proteins are Pichua (formerly classified as Hansenula), Saccharomyces, Kluyveromyces, Aspergillus, Candida, Torulopsis, Torulaspora, Schizosaccharomyces, Citeromyces, Pachysolen, Zygosaccharomyces, Debaromyces, Trichoderma, Cephalosporium, Humicola, Mucor, Neurospora, Yarrowia, Metschunikowia, Rhodosporidium, Leucosporidium, Botryoascus, Sporidiobolus, Endomycopsis, and the like. Preferred genera are those selected from the group consisting of Saccharomyces, Schizosaccharomyces, Kluyveromyces, Pichia and Torulaspora. Examples of Saccharomyces spp. are S. cerevisiae, S. italicus and S. rouxii.

Examples of Kluyveromyces spp. are K. fragilis, K. lactis and K. marxianus. A suitable Torulaspora species is T. delbrueckii. Examples of Pichia (Hansenula) spp. are P. angusta (formerly H. polymorpha), P. anomala (formerly H. anomala) and P. pastoris. Methods for the transformation of S. cerevisiae are taught generally in EP 251 744, EP 258 067 and WO 90/01063, all of which are incorporated herein by reference.

Preferred exemplary species of Saccharomyces include S. cerevisiae, S. italicus, S. diastaticus, and Zygosaccharomyces rouxii. Preferred exemplary species of Kluyveromyces include K. fragilis and K. lactis. Preferred exemplary species of Hansenula include H. polymorpha (now Pichia angusta), H. anomala (now Pichia anomala), and Pichia capsulata. Additional preferred exemplary species of Pichia include P. pastoris. Preferred exemplary species of Aspergillus include A. niger and A. nidulans. Preferred exemplary species of Yarrowia include Y. lipolytica. Many preferred yeast species are available from the ATCC. For example, the following preferred yeast species are available from the ATCC and are useful in the expression of albumin fusion proteins: Saccharomyces cerevisiae, Hansen, teleomorph strain BY4743 yap3 mutant (ATCC Accession No. 4022731); Saccharomyces cerevisiae Hansen, teleomorph strain BY4743 hsp150 mutant (ATCC Accession No. 4021266); Saccharomyces cerevisiae Hansen, teleomorph strain BY4743 pmt1 mutant (ATCC Accession No. 4023792); Saccharomyces cerevisiae Hansen, teleomorph (ATCC Accession Nos. 20626; 44773; 44774; and 62995); Saccharomyces diastaticus Andrews et Gilliland ex van der Walt, teleomorph (ATCC Accession No. 62987); Kluyveromyces lactis (Dombrowski) van der Walt, teleomorph (ATCC Accession No. 76492); Pichia angusta (Teunisson et al.) Kurtzman, teleomorph deposited as Hansenula polymorpha de Morais et Maia, teleomorph (ATCC Accession No. 26012); Aspergillus niger van Tieghem, anamorph (ATCC Accession No. 9029); Aspergillus niger van Tieghem, anamorph (ATCC Accession No. 16404); Aspergillus nidulans (Eidam) Winter, anamorph (ATCC Accession No. 48756); and Yarrowia lipolytica (Wickerham et al.) van der Walt et von Arx, teleomorph (ATCC Accession No. 201847).

Suitable promoters for S. cerevisiae include those associated with the PGKI gene, GAL1 or GAL10 genes, CYC1, PH05, TRP1, ADH1, ADH2, the genes for glyceraldehyde-3-phosphate dehydrogenase, hexokinase, pyruvate decarboxylase, phosphofructokinase, triose phosphate isomerase, phosphoglucose isomerase, glucokinase, alpha-mating factor pheromone, [a mating factor pheromone], the PRBI promoter, the GUT2 promoter, the GPDI promoter, and hybrid promoters involving hybrids of parts of 5′ regulatory regions with parts of 5′ regulatory regions of other promoters or with upstream activation sites (e.g. the promoter of EP-A-258 067).

Convenient regulatable promoters for use in Schizosaccharomyces pombe are the thiamine-repressible promoter from the nmt gene as described by Maundrell (1990) J. Biol. Chem. 265, 10857-10864 and the glucose repressible jbpl gene promoter as described by Hoffman & Winston (1990) Genetics 124, 807-816.

Methods of transforming Pichia for expression of foreign genes are taught in, for example, Gregg et al. (1993), and various Phillips patents (e.g. U.S. Pat. No. 4,857,467, incorporated herein by reference), and Pichia expression kits are commercially available from Invitrogen BV, Leek, Netherlands, and Invitrogen Corp., San Diego, Calif. Suitable promoters include AOX1 and AOX2. Gleeson et al. (1986) J. Gen. Microbiol. 132, 3459-3465 include information on Hansenula vectors and transformation, suitable promoters being MOX1 and FMD1; whilst EP 361 991, Fleer et al. (1991) and other publications from Rhone-Poulenc Rorer teach how to express foreign proteins in Kluyveromyces spp., a suitable promoter being PGKI.

The transcription termination signal is preferably the 3′ flanking sequence of a eukaryotic gene which contains proper signals for transcription termination and polyadenylation. Suitable 3′ flanking sequences may, for example, be those of the gene naturally linked to the expression control sequence used, i.e. may correspond to the promoter. Alternatively, they may be different in which case the termination signal of the S. cerevisiae ADHI gene is preferred.

In certain embodiments, the desired immunoglobulin construct protein is initially expressed with a secretion leader sequence, which may be any leader effective in the yeast chosen. Leaders useful in S. cerevisiae include that from the mating factor alpha polypeptide (MFα-1) and the hybrid leaders of EP-A-387 319. Such leaders (or signals) are cleaved by the yeast before the mature albumin is released into the surrounding medium. Further such leaders include those of S. cerevisiae invertase (SUC2) disclosed in JP 62-096086 (granted as 911036516), acid phosphatase (PH05), the pre-sequence of MF□-1, 0 glucanase (BGL2) and killer toxin; S. diastaticus glucoamylase II; S. carlsbergensis β-galactosidase (MEL1); K. lactis killer toxin; and Candida glucoamylase.

Provided are vectors containing a polynucleotide encoding an immunoglobulin construct described herein, host cells, and the production of the immunoglobulin constructs by synthetic and recombinant techniques. The vector may be, for example, a phage, plasmid, viral, or retroviral vector. Retroviral vectors may be replication competent or replication defective. In the latter case, viral propagation generally will occur only in complementing host cells.

In certain embodiments, the polynucleotides encoding immunoglobulin constructs described herein are joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

In certain embodiments, the polynucleotide insert is operatively linked to an appropriate promoter, such as the phage lambda PL promoter, the E. coli lac, trp, phoA and rac promoters, the SV40 early and late promoters and promoters of retroviral LTRs, to name a few. Other suitable promoters will be known to the skilled artisan. The expression constructs will further contain sites for transcription initiation, termination, and, in the transcribed region, a ribosome binding site for translation. The coding portion of the transcripts expressed by the constructs will preferably include a translation initiating codon at the beginning and a termination codon (UAA, UGA or UAG) appropriately positioned at the end of the polypeptide to be translated.

As indicated, the expression vectors will preferably include at least one selectable marker. Such markers include dihydrofolate reductase, G418, glutamine synthase, or neomycin resistance for eukaryotic cell culture, and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells (e.g., Saccharomyces cerevisiae or Pichia pastoris (ATCC Accession No. 201178)); insect cells such as Drosophila S2 and Spodoptera Sf9 cells; animal cells such as CHO, COS, NSO, 293, and Bowes melanoma cells; and plant cells. Appropriate culture mediums and conditions for the above-described host cells are known in the art.

Among vectors preferred for use in bacteria include pQE70, pQE60 and pQE-9, available from QIAGEN, Inc.; pBluescript vectors, Phagescript vectors, pNH8A, pNH16a, pNH18A; pNH46A, available from Stratagene Cloning Systems, Inc.; and ptrc99a, pKK223-3, pKK233-3, pDR540, pRIT5 available from Pharmacia Biotech, Inc. Among preferred eukaryotic vectors are pWLNEO, pSV2CAT, pOG44, pXT1 and pSG available from Stratagene; and pSVK3, pBPV, pMSG and pSVL available from Pharmacia. Preferred expression vectors for use in yeast systems include, but are not limited to pYES2, pYD1, pTEF1/Zeo, pYES2/GS, pPICZ, pGAPZ, pGAPZalph, pPIC9, pPIC3.5, pHIL-D2, pHIL-S1, pPIC3.5K, pPIC9K, and PAO815 (all available from Invitrogen, Carlbad, Calif.). Other suitable vectors will be readily apparent to the skilled artisan.

In one embodiment, polynucleotides encoding an immunoglobulin construct described herein are fused to signal sequences that will direct the localization of a protein of the invention to particular compartments of a prokaryotic or eukaryotic cell and/or direct the secretion of a protein of the invention from a prokaryotic or eukaryotic cell. For example, in E. coli, one may wish to direct the expression of the protein to the periplasmic space. Examples of signal sequences or proteins (or fragments thereof) to which The immunoglobulin constructs are fused in order to direct the expression of the polypeptide to the periplasmic space of bacteria include, but are not limited to, the pelB signal sequence, the maltose binding protein (MBP) signal sequence, MBP, the ompA signal sequence, the signal sequence of the periplasmic E. coli heat-labile enterotoxin B-subunit, and the signal sequence of alkaline phosphatase. Several vectors are commercially available for the construction of fusion proteins which will direct the localization of a protein, such as the pMAL series of vectors (particularly the pMAL-.rho. series) available from New England Biolabs. In a specific embodiment, polynucleotides albumin fusion proteins of the invention may be fused to the pelB pectate lyase signal sequence to increase the efficiency of expression and purification of such polypeptides in Gram-negative bacteria. See, U.S. Pat. Nos. 5,576,195 and 5,846,818, the contents of which are herein incorporated by reference in their entireties.

Examples of signal peptides that are fused to an immunoglobulin construct in order to direct its secretion in mammalian cells include, but are not limited to, the MPIF-1 signal sequence (e.g., amino acids 1-21 of GenBank Accession number AAB51134), the stanniocalcin signal sequence (MLQNSAVLLLLVISASA), and a consensus signal sequence (MPTWAWWLFLVLLLALWAPARG). A suitable signal sequence that may be used in conjunction with baculoviral expression systems is the gp67 signal sequence (e.g., amino acids 1-19 of GenBank Accession Number AAA72759).

Vectors which use glutamine synthase (GS) or DHFR as the selectable markers can be amplified in the presence of the drugs methionine sulphoximine or methotrexate, respectively. An advantage of glutamine synthase based vectors are the availability of cell lines (e.g., the murine myeloma cell line, NSO) which are glutamine synthase negative. Glutamine synthase expression systems can also function in glutamine synthase expressing cells (e.g., Chinese Hamster Ovary (CHO) cells) by providing additional inhibitor to prevent the functioning of the endogenous gene. A glutamine synthase expression system and components thereof are detailed in PCT publications: WO87/04462; WO86/05807; WO89/10036; WO89/10404; and WO91/06657, which are hereby incorporated in their entireties by reference herein. Additionally, glutamine synthase expression vectors can be obtained from Lonza Biologics, Inc. (Portsmouth, N.H.). Expression and production of monoclonal antibodies using a GS expression system in murine myeloma cells is described in Bebbington et al., Bio/technology 10:169 (1992) and in Biblia and Robinson Biotechnol. Prog. 11:1 (1995) which are herein incorporated by reference.

Also provided are host cells containing vector constructs described herein, and additionally host cells containing nucleotide sequences that are operably associated with one or more heterologous control regions (e.g., promoter and/or enhancer) using techniques known of in the art. The host cell can be a higher eukaryotic cell, such as a mammalian cell (e.g., a human derived cell), or a lower eukaryotic cell, such as a yeast cell, or the host cell can be a prokaryotic cell, such as a bacterial cell. A host strain may be chosen which modulates the expression of the inserted gene sequences, or modifies and processes the gene product in the specific fashion desired. Expression from certain promoters can be elevated in the presence of certain inducers; thus expression of the genetically engineered polypeptide may be controlled. Furthermore, different host cells have characteristics and specific mechanisms for the translational and post-translational processing and modification (e.g., phosphorylation, cleavage) of proteins. Appropriate cell lines can be chosen to ensure the desired modifications and processing of the foreign protein expressed.

Introduction of the nucleic acids and nucleic acid constructs of the invention into the host cell can be effected by calcium phosphate transfection, DEAE-dextran mediated transfection, cationic lipid-mediated transfection, electroporation, transduction, infection, or other methods. Such methods are described in many standard laboratory manuals, such as Davis et al., Basic Methods In Molecular Biology (1986). It is specifically contemplated that the polypeptides of the present invention may in fact be expressed by a host cell lacking a recombinant vector.

In addition to encompassing host cells containing the vector constructs discussed herein, the invention also encompasses primary, secondary, and immortalized host cells of vertebrate origin, particularly mammalian origin, that have been engineered to delete or replace endogenous genetic material, and/or to include genetic material. The genetic material operably associated with the endogenous polynucleotide may activate, alter, and/or amplify endogenous polynucleotides.

In addition, techniques known in the art may be used to operably associate heterologous polynucleotides and/or heterologous control regions (e.g., promoter and/or enhancer) with endogenous polynucleotide sequences encoding a Therapeutic protein via homologous recombination (see, e.g., U.S. Pat. No. 5,641,670, issued Jun. 24, 1997; International Publication Number WO 96/29411; International Publication Number WO 94/12650; Koller et al., Proc. Natl. Acad. Sci. USA 86:8932-8935 (1989); and Zijlstra et al., Nature 342:435-438 (1989), the disclosures of each of which are incorporated by reference in their entireties).

Immunoglobulin constructs described herein can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography, hydrophobic charge interaction chromatography and lectin chromatography. Most preferably, high performance liquid chromatography (“HPLC”) is employed for purification.

In certain embodiments the immunoglobulin constructs of the invention are purified using Anion Exchange Chromatography including, but not limited to, chromatography on Q-sepharose, DEAE sepharose, poros HQ, poros DEAF, Toyopearl Q, Toyopearl QAE, Toyopearl DEAE, Resource/Source Q and DEAE, Fractogel Q and DEAE columns.

In specific embodiments the proteins described herein are purified using Cation Exchange Chromatography including, but not limited to, SP-sepharose, CM sepharose, poros HS, poros CM, Toyopearl SP, Toyopearl CM, Resource/Source S and CM, Fractogel S and CM columns and their equivalents and comparables.

In addition, immunoglobulin constructs described herein can be chemically synthesized using techniques known in the art (e.g., see Creighton, 1983, Proteins: Structures and Molecular Principles, W. H. Freeman & Co., N.Y and Hunkapiller et al., Nature, 310:105-111 (1984)). For example, a polypeptide corresponding to a fragment of a polypeptide can be synthesized by use of a peptide synthesizer. Furthermore, if desired, nonclassical amino acids or chemical amino acid analogs can be introduced as a substitution or addition into the polypeptide sequence. Non-classical amino acids include, but are not limited to, to the D-isomers of the common amino acids, 2,4diaminobutyric acid, alpha-amino isobutyric acid, 4-aminobutyric acid, Abu, 2-amino butyric acid, g-Abu, e-Ahx, 6amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, □-alanine, fluoro-amino acids, designer amino acids such as □-methyl amino acids, C□-methyl amino acids, N□-methyl amino acids, and amino acid analogs in general. Furthermore, the amino acid can be D (dextrorotary) or L (levorotary).

Provided are immunoglobulin constructs which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited, to specific chemical cleavage by cyanogen bromide, trypsin, chymotrypsin, papain, V8 protease, NaBH₄; acetylation, formylation, oxidation, reduction; metabolic synthesis in the presence of tunicamycin; etc.

Additional post-translational modifications encompassed herein include, for example, e.g., N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of procaryotic host cell expression. The immunoglobulin constructs are modified with a detectable label, such as an enzymatic, fluorescent, isotopic or affinity label to allow for detection and isolation of the protein.

Examples of suitable enzymes include horseradish peroxidase, alkaline phosphatase, beta-galactosidase, or acetylcholinesterase; examples of suitable prosthetic group complexes include streptavidin biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include iodine, carbon, sulfur, tritium, indium, technetium, thallium, gallium, palladium, molybdenum, xenon, fluorine.

In specific embodiments, immunoglobulin constructs or fragments or variants thereof are attached to macrocyclic chelators that associate with radiometal ions.

As mentioned, the immunoglobulin construct described herein is modified by either natural processes, such as post-translational processing, or by chemical modification techniques which are well known in the art. It will be appreciated that the same type of modification may be present in the same or varying degrees at several sites in a given polypeptide. Polypeptides of the invention may be branched, for example, as a result of ubiquitination, and they may be cyclic, with or without branching. Cyclic, branched, and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, PROTEINS—STRUCTURE AND MOLECULAR PROPERTIES, 2nd Ed., T. E. Creighton, W. H. Freeman and Company, New York (1993); POST-TRANSLATIONAL COVALENT MODIFICATION OF PROTEINS, B. C. Johnson, Ed., Academic Press, New York, pgs. 1-12 (1983); Seifter et al., Meth. Enzymol. 182:626-646 (1990); Rattan et al., Ann. N.Y. Acad. Sci. 663:48-62 (1992)).

In certain embodiments, immunoglobulin constructs may also be attached to solid supports, which are particularly useful for immunoassays or purification of polypeptides that are bound by, that bind to, or associate with immunoglobulin constructs described herein. Such solid supports include, but are not limited to, glass, cellulose, polyacrylamide, nylon, polystyrene, polyvinyl chloride or polypropylene.

### EXAMPLES

The following specific and non-limiting examples are to be construed as merely illustrative, and do not limit the present disclosure in any way whatsoever. Without further elaboration, it is believed that one skilled in the art can, based on the description herein, utilize the present disclosure to its fullest extent. All publications cited herein are hereby incorporated by reference in their entirety. Where reference is made to a URL or other such identifier or address, it is understood that such identifiers can change and particular information on the internet can come and go, but equivalent information can be found by searching the internet. Reference thereto evidences the availability and public dissemination of such information.

### Example 1

### Linker Composition

The linker peptide connecting domains in single chain format can influence properties of designed protein such as proteolytic stability, conformational stability, refolding kinetics, extent of multimerization and antigen affinity. The long linker designs described here were identified by testing several linker lengths and compositions in the context of scFab and the scFvs connected to heavy chain domains as presented in Table 1. While the GGGS sequence is commonly used, charge can be introduced in the linker to provide altered hydrodynamic properties and some of these linkers comprise of sequences that have a propensity to form helical secondary structure. Polypeptide sequences that preferentially form helical structure are known in the art [Marqusee, S, and Baldwin, R. L. (1987) Proc. Natl. Acad. Sci. USA, 84, 8898-8902]. These polypeptides typically have residues that favourably interact with each other at the i and (i+4)^{th} position in the polypeptide sequence. The constructs described herein present the unique and unexpected advantage that the use of such helix forming polypeptides provides in the design and creation of Fab molecules in single chain format. For scFabs in the long linker format, the long linker connects the CL and VH domains. Table 1 shows the linkers used.

TABLE 1
Linkers used
Linker Composition

(GₗSₘ)ₙ; l = 0, 1, 2, 3, 4, 5; m = 0, 1; n = 5, 6,
7, . . .
(SₚG_{q})ₗ(SEG_{q})ₘ(SₚG_{q})ᵣ; l, m = 2, 3, 4, . . . ; p,
q, r = 0, 1, 2, 3, . . .
GSTSGSGGSTSGSGKPGSGEGSTKGGSTSGSG
GGSGGGSGSSADDAKKDAAKKDDAKKDDAKKDGGGSGGGSG

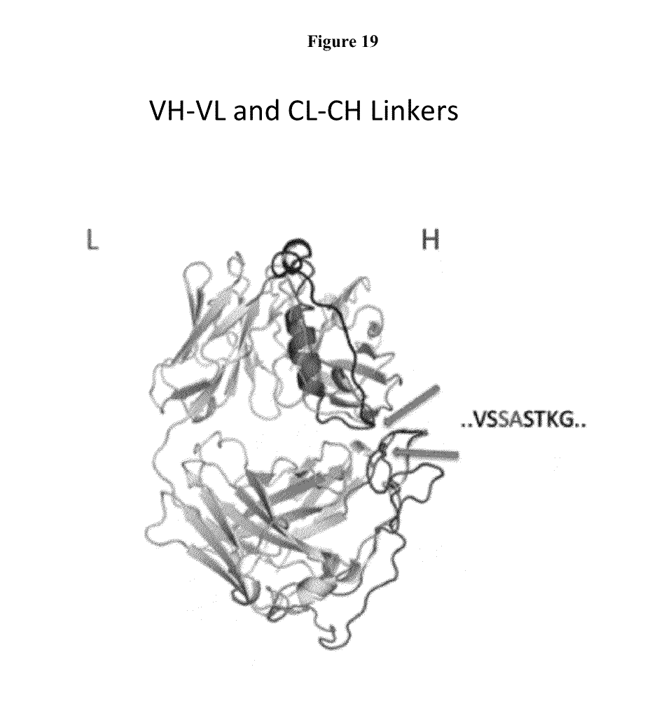

The Light Chain Insert design as described herein refers to the design of immunoglobulin constructs comprising single chain Fv polypeptides connected to constant domain polypeptides of the immunoglobulin chain. The light chain insert design comprises of shorter linkers at two different locations, one connecting the VH and VL domains (VHVL linker) and another connecting the CH and CL domains (CHCL linker). Molecular modeling was employed to propose linker lengths. FIG. 19 shows a typical model of the light chain insert format.

Table 2 shows the linkers used in the Light Chain Insert design.

TABLE 2
Linkers used for Light Chain Insert design. In
some embodiments other residues at the N and/or C
terminus may be introduced to cap these linker
residues.
Linker Composition

(GₗSₘ)ₙ; l = 0, 1, 2, 3, 4, 5; m = 0, 1; n = 3, 4,
5, . . .
GₗSₘGSTSGSGKPGSGEGSTKGGₙSₒ;
l, m, n, n, o = 0, 1, 2, . . .

As expected, due to high linker flexibility, there is no electron density corresponding to VHVL linkers in available structures of scFvs. The relative location of the C terminus of VH and N terminus of VL suggest that linker path would be similar to as the one modeled in MOE, being in proximity to the VH:VL interface.

### Example 2

### Preparation of scFab Constructs Using a Long Linker (LL) Strategy or scFv by Use of Light Chain Insert (LCI) Strategy

Sequence of the antibody D3H44 was extracted from the 1JPS structure in PDB (Tissue factor in complex with humanized D3H44Fab). Similarly, the sequence of Antibody 4D5 was obtained from the 1N8Z structure (complex of extracellular region of HER2 and herceptin Fab). The sequence of the NM3E2 (anti-CD16) scFv was obtained from the literature [Isolation and characterization of an anti-CD16 single-chain Fv fragment and construction of an anti-HER2/neu/anti-CD16 bispecific scFv that triggers CD16-dependent tumor cytolysis. McCall et al. 1999, Mol Immunol, 36(7):433-445]. Single chain Fab and light chain insert structures were designed by linking the domains with the linkers listed in tables 1 and 2 above. These constructs were prepared using standard recombinant DNA technology. For example, for the scFabs with a long linker, The specific designs for the long linker constructs are shown in Table 3 and the specific designs for the LCI constructs are shown in Table 4 below.

In DNA space, all sequences have preceding signal sequence corresponding to MAVMAPRTLVLLLSGALALTQTWAG and restriction sites, at 5′ EcoRI and 3′ BamHI. All Fabs contain cysteine residues necessary for disulfide bond formation between CL and CH1. In case of LCI insert, linkers were attached to newly created ends in VH and CH1 domains in such a manner as to mimic scFv on one end and to avoid clashes between linkers at the other end aided with removal of one dispensable amino acid residue at the newly created N-terminus of CH1. The scFab format constructs comprise of the VH, VL, CL, CH1 domains with the appropriate linkers indicated.

TABLE 3
Linker sequences and legend for scFabs

| type | | | | | | helical- |
|---|---|---|---|---|---|---|
| of Fab | GS-30 | GS-35 | GSE-30 | GSE-34 | GST-32 | 41 |
| 4D5 | v638 | v639 | v641 | v640 | v655 | v654 |
| D3H44 | v657 | v658 | v660 | v659 | v674 | v673 |

Linker legend:
GS-30: (GGGGS)6
GS-35: (GGGGS)7
GSE-30: (SGGG)2(SEGGG)4SG
GSE-34: (SGGG)2(SEGGG)4SGGGSG
GST-32: GSTSGSGGSTSGSGKPGSGEGSTKGGSTSGSG
Helical-41: GGSGGGSGSSADDAKKDAAKKDDAKKDDAKKDGGGSGGGSG

TABLE 4
Linker sequences and legend for additional scFabs

| scFab | VHVL_linker | CLCH_linker |
|---|---|---|
| v642-4D5 | GS-15 | GS-20 |
| v643-4DS, v662-D3H44 | GS-15 | GS-24 |
| v644-4D5 | GS-15 | GS-28 |
| v664-D3H44 | GS-20 | GS-20 |
| v646-4D5, v665-D3H44 | GS-20 | GS-24 |
| v-666-D3H44, v647-4D5 | GS-20 | GS-28 |
| v648-4D5 | GST-18 | GS-20 |
| v649-4D5 | GST-18 | GS-24 |
| v669-D3H44 | GST-18 | GS-28 |
| v651-4D5 | GST-20 | GS-20 |
| v671-D3H44 | GST-20 | GS-24 |
| v672-D3H44 | GST-20 | GS-28 |
| V656-4D5, v675-D3H44 | GST-18 | GST-26 |

Linker legend:
GS-15: (GGGGS)3
GS-20: (GGGGS)4
GS-24: (GGGGS)4GGGG
GS-28: (GGGGS)5GGG
GS-30: (GGGGS)6
GST-18: GSTSGSGKPGSGEGSTKG
GST-20: GSTSGSGKPGSGEGSTKGSG
GST-26: GSTSGSTSGSGKPGSGEGSTKGGSTS
GSE-30: (SGGG)2(SEGGG)4SG
GSE-34: (SGGG)2(SEGGG)4SGGGSG

### Example 3

### Expression, Purification and Analysis of scFabs

Expression was performed in 2 mL HEK293 (in triplicate). Cells were transfected in exponential growth phase (1.5 to 2 millions cells/ml) with PEI (Polyethylenimine linear 25 kDa dissolve in water to 1 mg/ml, Polysciences, cat#23966) and 1 ug DNA/ml of cells at a ratio PEI/DNA of 2.5:1. Salmon sperm DNA (70%) is added to complete 100 ug DNA. PEI is mixed to transfection medium in 1/20 volume of total transfection. The PEI/DNA mixture is vortexed and incubated at RT for 3 minutes. Transfection medium (pre-warmed at room temperature or 37° C.) is the same as that used for maintenance of cells (F17 media supplemented with 4 mM L-Glutamine, 0.1% Pluronic F68 and 0.025 mg/ml G418.). Expression was assessed by SDS-PAGE 4-12% gradient gels under reducing or non-reducing conditions, no boiling, using MOPS buffer.

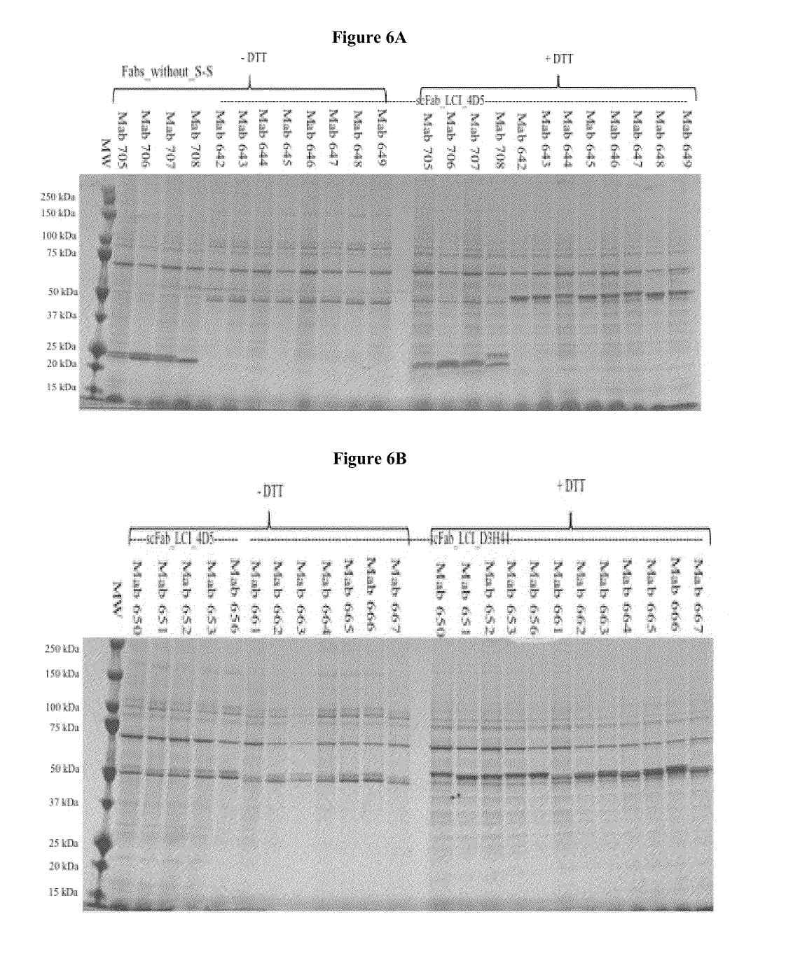

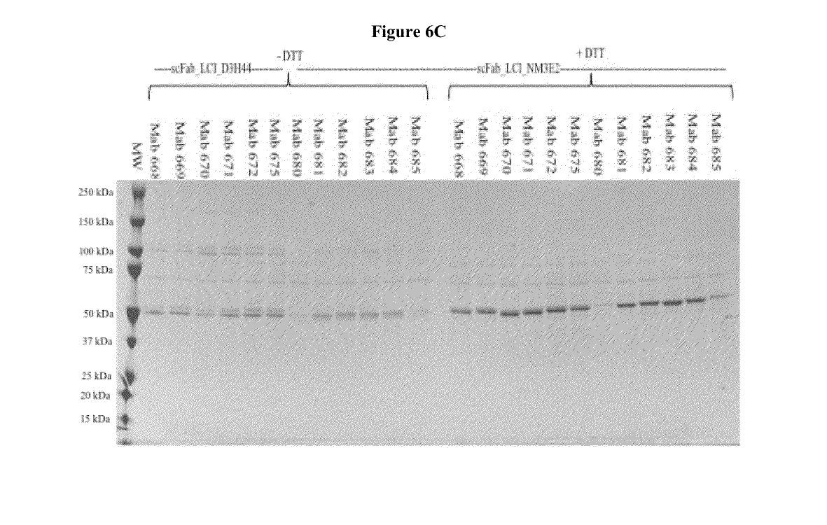

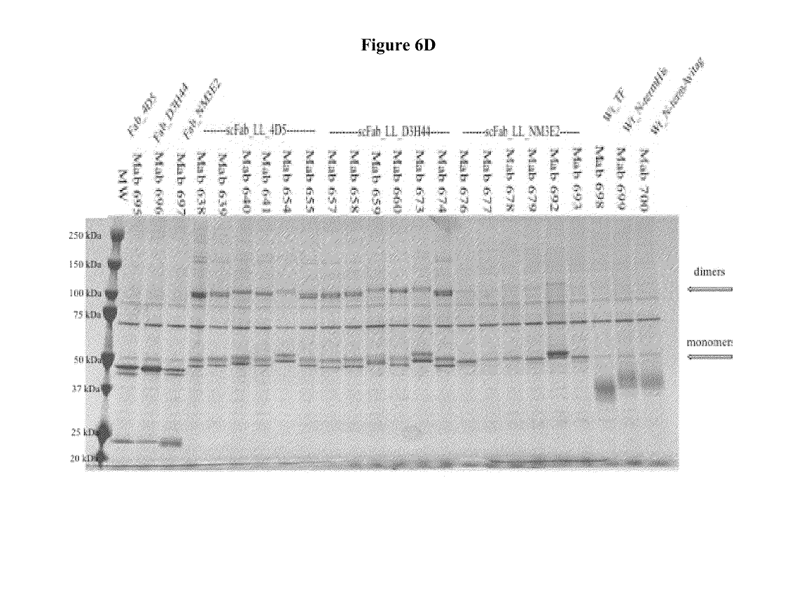

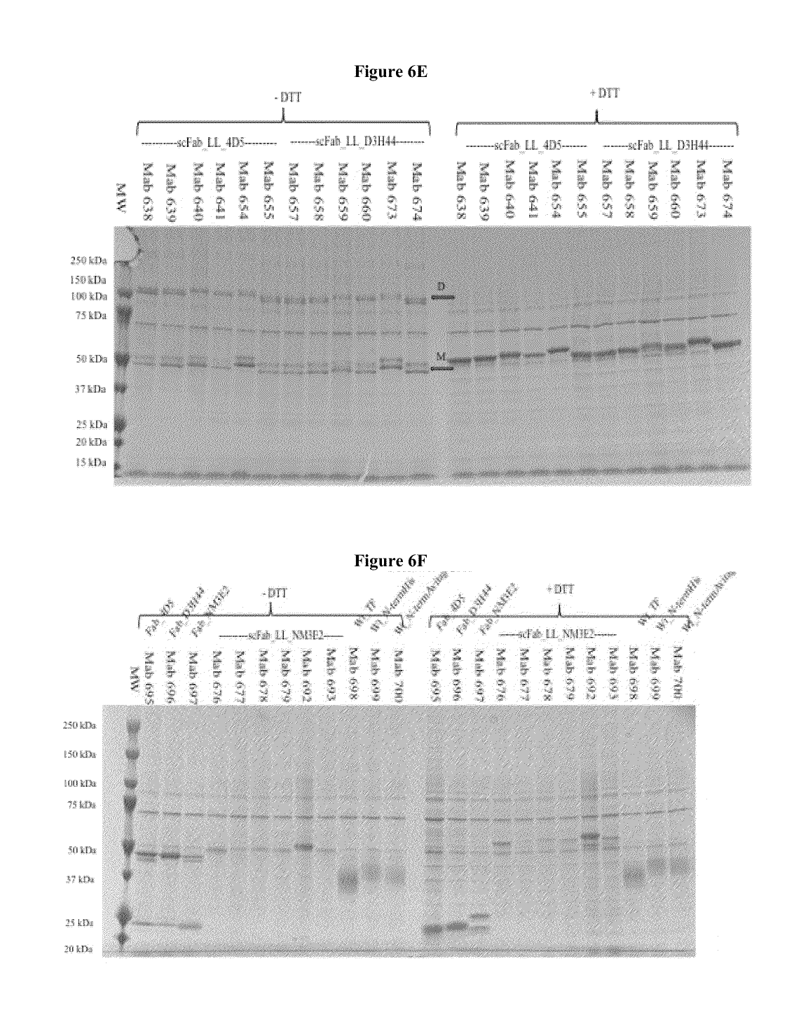

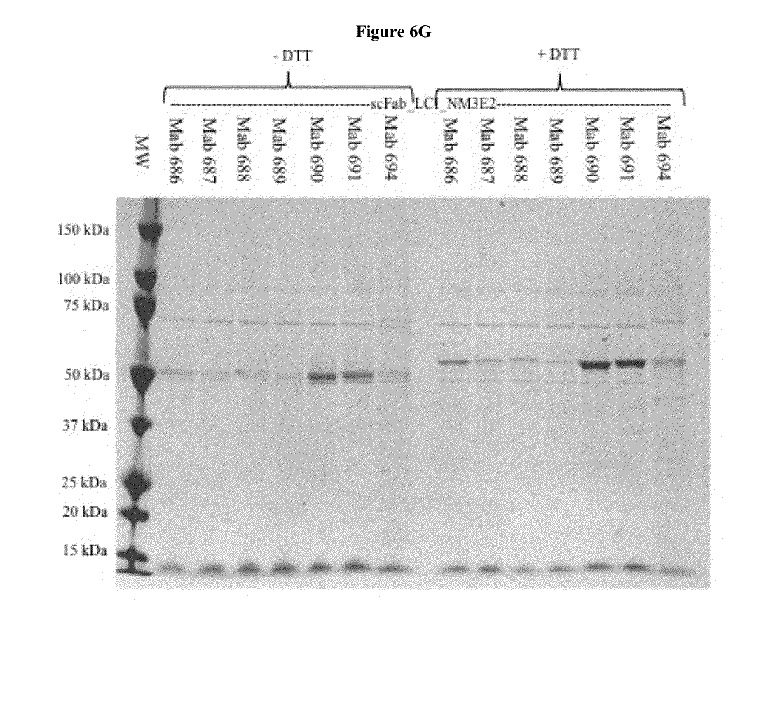

Expression results are shown in FIGS. 6A to F. FIG. 6A shows the expression of Fabs with no disulphide, scFab 4D5 with light chain inserts; FIG. 6B shows the expression of scFab 4D5, scFab D3H44 with linker inserts; FIG. 6C shows the expression of scFab D3H44 and scFab NM3E2 with linker inserts; FIG. 6D shows the expression of Fab controls, scFab 4D5, scFab D3H44, scFab NM3E2 and TF in the absence of DTT; FIG. 6E shows the expression of scFab 4D5, scFab D3H44; FIG. 6F shows the expression of Fab controls, scFab NM3E2 and TF; and FIG. 6G shows the expression of scFab NM3E2 with linker inserts. The band close to 50 kDa in FIGS. 6A, 6B and 6C indicate the formation of scFab's in the light chain insert format. In FIGS. 6D, 6E, 6F and 6G a comparison of scFab expression with various types of long linkers is presented. While expression of the expected monomer species is observed in most cases, the level of expression and level of monomer observed relative to dimeric species formed indicate that some linkers perform better than others. The variants v654 and v673 depicting scFab variants with the helical linker (H41) binding different antigen targets, tend to consistently express better with lower amounts of dimers being formed in Fab's.

Scale-Up and Purification

Samples were scaled up to 500 mL HEK 293 cells. The expressed protein in supernatant was concentrated to 125 mL and loaded onto KappaSelect affinity column at flow rate of 1 ml/min. Equilibration and wash was performed with 10CV of PBS buffer followed by elution of the protein with 0.1M glycine at pH 3.0. Pool fractionation and desalting was performed on Econo-Pac column and the protein stored in PBS.

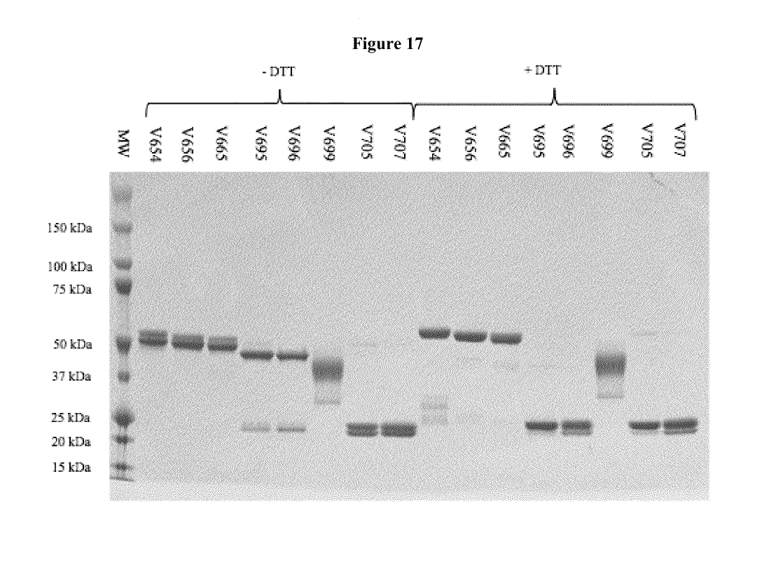

Protein yield was estimated via nanodrop. SDS-PAGE was run in non-reducing and reducing conditions (FIG. 17). The non-reducing gel indicates multimerization if present. Control molecule v695 (4D5 Fab) without the linker runs with the expected MW of ˜50 kDa, showing weak disulfide reduction, evident from the band at 25 kDa.

A summary of the results of scale-up and protein purification are shown in Table 5 below.

TABLE 5
Summary of variant yield, post-affinity purification.

| | | Final purified | Final concentration | |
|---|---|---|---|---|
| | | Volume | (mg/ml) and total | |
| Variant | Type | (ml) | mg, post-affinity | |
| 695 | Fab 4D5 (control) | 4 | 1.27 | (5.08 mg) |
| 696 | Fab D3H44 (control) | 8 | 1.8 | (14.4 mg) |
| 654 | 4D5 scFab (CL-VH | 8 | 0.66 | (6.88 mg) |
| | linker) | | | |
| 656 | 4D5 scFab (LC insert) | 8 | 2.66 | (21.28 mg) |
| 665 | D3H44 scFab (LC | 12 | 1.14 | (13.68 mg) |
| | insert) | | | |
| 673 | D3H44 scFab (CL-VH | 0.25 | 2.44 | (0.6 mg) |
| | linker) | | | |
| 705 | 4D5 Fab no H-L | 4 | 2.31 | (9.2 mg) |
| | disulfide | | | |
| | (C214S/C223S) | | | |
| 707 | D3H44 Fab no H-L | 8 | 2.03 | (16.2 mg) |
| | disulfide | | | |
| | (C214A/C220A) | | | |

The product single chain Fab (scFab) obtained for two different antigen binding Fab's (4D5 and D3H44) in both the LCI and LL format are comparable to that obtained without the linkers.

SEC (Size Exclusion Chromatography)

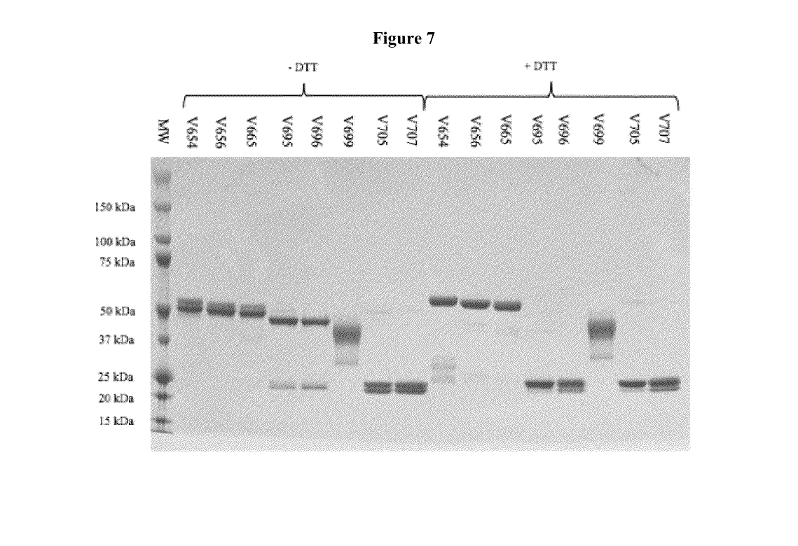

Variants were purified by SEC according to a standard protocol, employing a Superdex S200 (16/60) following manufacturer instructions. Running buffer was PBS. The purified proteins were assessed by SDS-PAGE. The SDS-PAGE gel results are shown in FIG. 7. A summary of the results is shown in Table 6 below.

TABLE 6
Summary of SEC purification.

| | | | | Protein | | |
|---|---|---|---|---|---|---|
| | | | | loaded | | Final conc. |
| | | Volume | Final | on gel | Volume | mg/ml after |
| | | PBS | Concentration | SEC | PBS | GFC |
| | | before | mg/ml | column | after | (monomeric |
| Variant | Type | GFC | before GFC | (mg) | GFC | fractions) |
| 695 | Fab 4D5 | 4 | 1.27 (5.08 mg) | 2.5 mg | 1.4 | 0.56 (0.784 mg) |
| | (control) | | | | | |
| 696 | Fab D3H44 | 8 | 1.8 (14.4 mg) | 7.2 mg | 1.5 | 1.4 (2.1 mg) |
| | (control) | | | | | |
| 654 | 4D5 scFab | 8 | 0.86 (6.88 mg) | 3.4 mg | 1.25 | 0.66 (0.825 mg) |
| | (VH-CL | | | | | |
| | linker) | | | | | |
| 656 | 4D5 scFab | 8 | 2.66 (21.28 mg) | 10.6 mg | 1.5 | 1 (1.5 mg) |
| | (LC insert) | | | | | |
| 665 | D3H44 | 12 | 1.14 (13.68 mg) | 6.8 mg | 1.35 | 0.9 (1.2 mg) |
| | scFab (LC | | | | | |
| | insert) | | | | | |
| 673 | D3H44 | 0.25 | 2.44 (0.610 mg) | 0.3 mg | | ND |
| | scFab (VH- | | | | | |
| | CL linker) | | | | | |
| 699 | His-TF | 8 | 3.62 (28.96 mg) | 14.5 mg | 5 | 1.15 (5.75 mg) |
| | (antigen) | | | | | |
| 705 | 4D5 Fab no | 4 | 2.31 (9.24 mg) | 4.6 mg | 1.5 | 0.63 (1.1 mg) |
| | H-L disulfide | | | | | |
| | (C214S/C223S) | | | | | |
| 707 | D3H44 Fab | 8 | 2.03 (16.24 mg) | 8.1 mg | 3.5 | 0.76 (2.66 mg) |
| | no H-L | | | | | |
| | disulfide | | | | | |
| | (C214A/C220A) | | | | | |

Binding

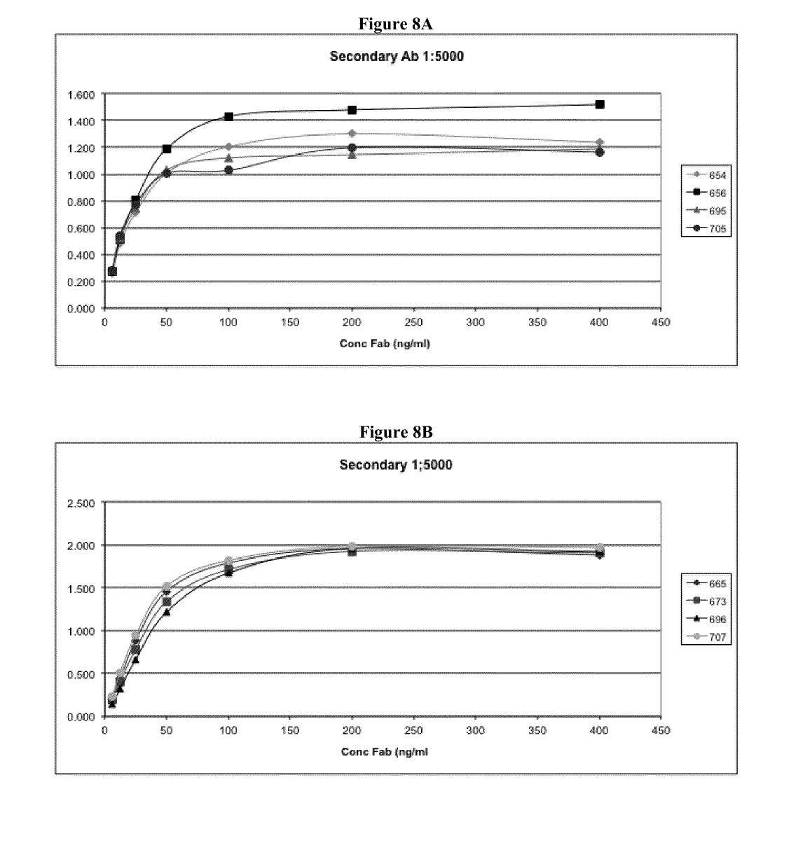

SPR and an ELISA-based antigen binding assay was performed on SEC purified variants to establish that the variants were able to bind to the target antigen. For SPR the scFabs were captured on a chip saturated with anti hIgG and ligands (Her2 or TF) was flowed over the chip at 300 nM. For the ELISA assay, 0.5 ug/ml Her2 or 10 ug/ml-0.156 ug/ml TF was placed on the assay plate in PBS. Single chain Fab variants were serially diluted (in 1:2 dilution steps) from 400 ng/ml to 6.25 ng/ml. Detection was performed using goat (Fab′)2 anti-human (Fab′)2 fragment specific at 1:5000, 1:10,000 and 1:20,000 dilutions. FIGS. 8A-8B show the results of the ELISA-based antigen binding assay for variants 654, 658, 695, and 705 (FIG. 8A), as well as variants 685, 673, 696, and 707 (FIG. 8B). Table 7 below depicts the binding data obtained using SPR.

TABLE 7
Binding data for scFab variants by SPR

| Variant | Description | ka (1/Ms) | kd (1/s) | K_{D} (M) |
|---|---|---|---|---|
| 665 | scFab + linker A2_A2 | 3.18E+06 | 1.59E−04 | 5.00E−11 |
| 673 | scFab + long linker D1 | 2.78E+06 | 1.27E−04 | 4.55E−11 |
| 707 | D3H44 Cys to Ala no | 3.08E+06 | 1.16E−04 | 3.78E−11 |
| | disulfide | | | |
| 696 | D3H44 WT | 3.21E+06 | 4.86E−05 | 1.52E−11 |

Note that the binding affinity of both the Her2 and tissue factor binding Fab's (4D5 Fab and D3H44Fab) when constructed in the single chain format retain parent Fab like antigen biding affinity.

Stability

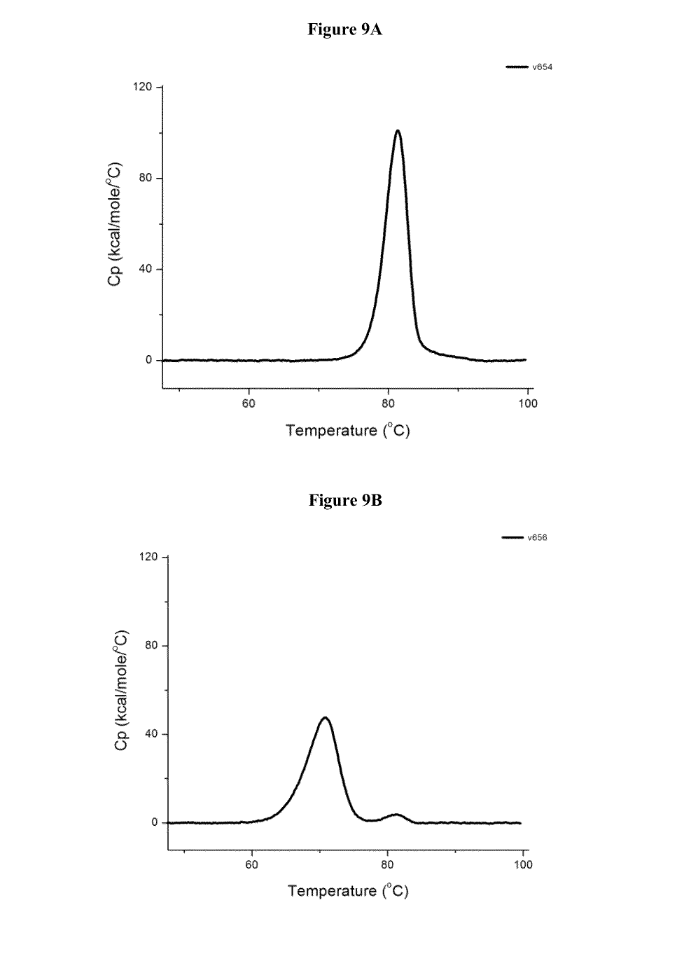

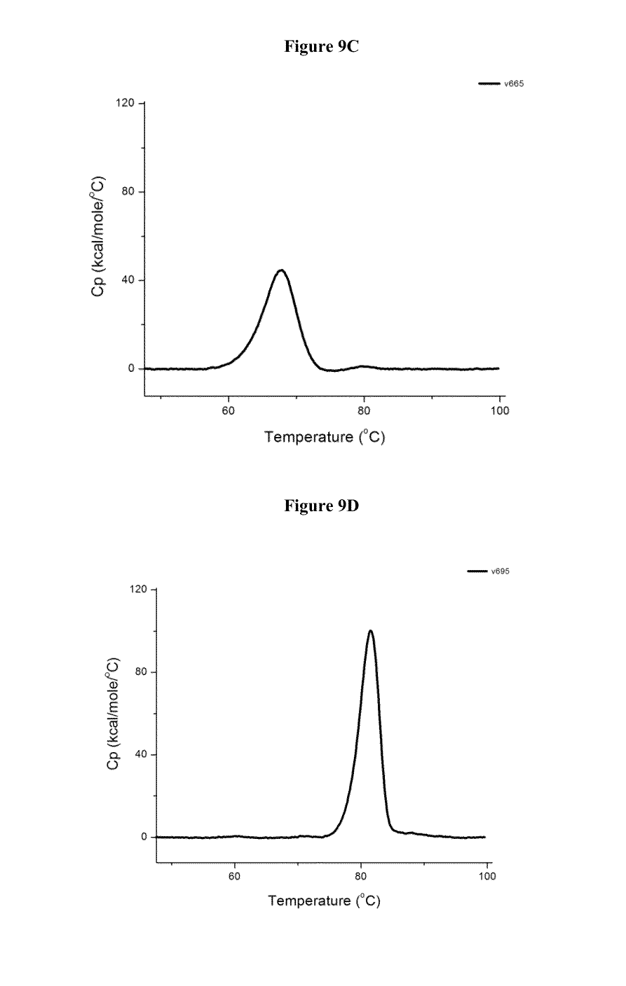

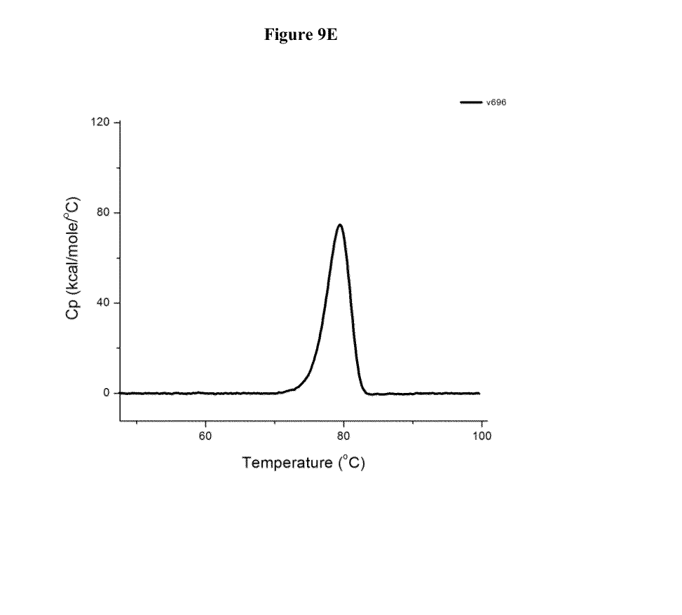

Differential scanning calorimetry (DSC) was performed on SEC purified variants to evaluate thermodynamic stability of the molecule (FIG. 9A-E). DSC experiments were carried out using a GE or MicroCal VP-Capillary instrument. The proteins were buffer-exchanged into PBS (pH 7.4) and diluted to 0.3 to 0.7 mg/mL with 0.137 mL loaded into the sample cell and measured with a scan rate of 1° C./min from 20 to 100° C. Data was analyzed using the Origin software (GE Healthcare) with the PBS buffer background subtracted.

A summary of the DSC results is found in Table 8 below.

TABLE 8
DSC results for scFab variants

| variant # | scFab format | system | Tm (° C.) |
|---|---|---|---|
| 695 | native Fab | HER2 (4D5) | 81.2 |
| 696 | native Fab | TF (D3H44) | 79.1 |
| 654 | scFab LL | HER2 (4D5) | 80.2 |
| 656 | scFab LCI | HER2 (4D5) | 70.3 |
| 673 | scFab LL | TF (D3H44) | 78.9 |
| 665 | scFab LCI | TF (D3H44) | 67.4 |
| 705 | Fab, no S-S | HER2 (4D5) | 78.3 |
| 707 | Fab, no S-S | TF (D3H44) | 77.7 |

The variant Fabs in the single chain format with the long linker retain parent Fab-like thermal stability. The scFab in the light chain insert format have about 10° C. lower thermal stability relative to the parent Fab.

### Example 4

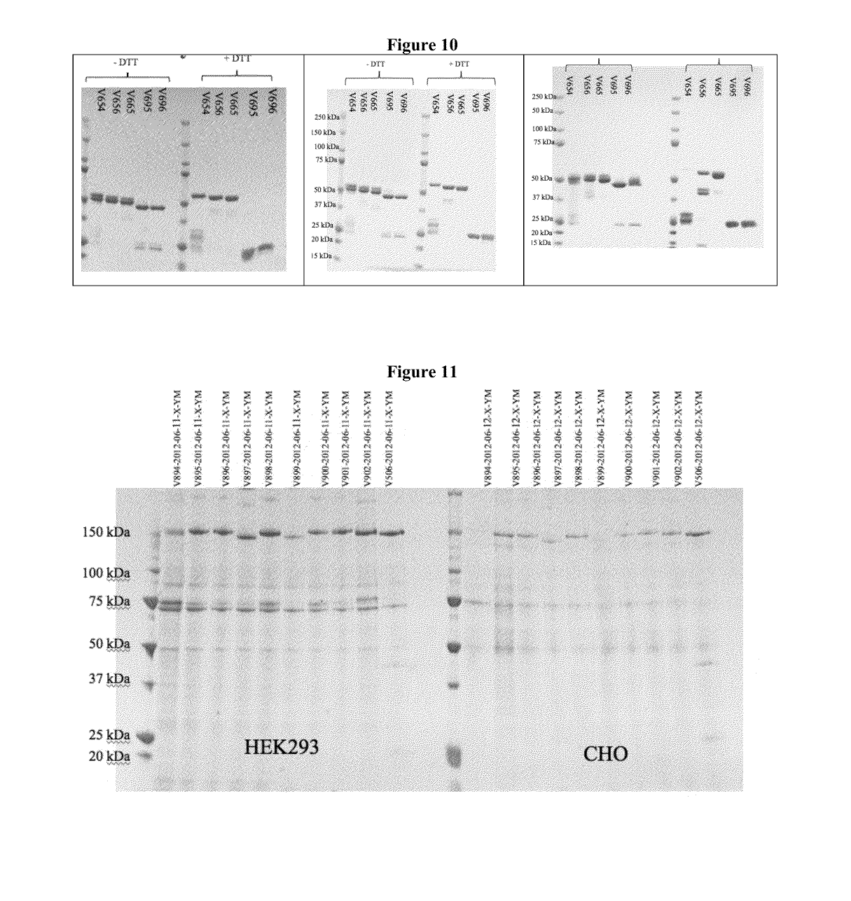

### Benchtop Stability Assay of Single Chain Fab Format (FIG. 10)

The benchtop stability test consisted of taking a sample of each protein variant under analysis and monitoring breakdown and/or oligomerization over the course of a week-long storage at room temperature (20 C). Assessment was carried out using reducing and non-reducing SDS-PAGE, loading 2.5 □g of protein per well, followed by Size Exclusion chromatography (SEC) and UPLC. Protein concentration was determined by A280 nanodrop. Each protein sample consists of size-exclusion purified protein corresponding to the monomeric fraction. Proteins were kept in vials at room temperature (benchtop), with an initial sample taken at the beginning of the experiment (time 0). Subsequent samples were taken after 24 hours, 3 days and 7 days. Each sample was denatured in Commassie Blue SDS buffer and stored at −80° C. until final SDS-PAGE assessment.

The results of SDS-PAGE analysis of the samples is shown in FIG. 10A (1 day), FIG. 10B (3 days) and FIG. 10C (7 days). A summary of the results is provided in Tables 9 and 10 below.

TABLE 9
Summary of benchtop stability results

| | scFab | | Tm | Day 1 - | Day 3 - | Day 7 - | |
|---|---|---|---|---|---|---|---|
| variant # | format | system | (° C.) | UPLC | UPLC | UPLC | SEC |
| 695 | native | HER2 | 81.2 | stable | stable | stable | stable |
| | Fab | (4D5) | | monomeric | monomeric | monomeric | monomeric |
| 696 | native | TF | 79.1 | stable | stable | stable | stable |
| | Fab | (D3H44) | | monomeric | monomeric | monomeric | monomeric |
| 654 | scFab | HER2 | 80.2 | two peaks | Equilibrium | conformational | Monomeric |
| | LL | (4D5) | | visible at | of different | changes | peak |
| | | | | 3.35 and | species. | (UPLC) | consistent |
| | | | | 3.55 min. | conformational | | throughout the |
| | | | | Both peaks | changes | | 7 days |
| | | | | are | (UPLC) | | |
| | | | | consistent | | | |
| | | | | with a | | | |
| | | | | monomeric | | | |
| | | | | specie but | | | |
| | | | | likely | | | |
| | | | | represent | | | |
| | | | | two different | | | |
| | | | | conformations. | | | |
| 656 | scFab | HER2 | 70.3 | Mainly | Mainly | Mainly | Mainly |
| | LCI | (4D5) | | monomeric. | monomeric. | monomeric. | monomeric and |
| | | | | Dimer peak | Dimer | No | stable |
| | | | | visible at | peak | dimer | throughout the |
| | | | | 3.14 min; | disappears. | peak. | 7 days. |
| | | | | main peak | Leading | Leading | Secondary |
| | | | | encompasses | shoulder | shoulder | peak visible; 5% |
| | | | | 95% of | on | on | of total area |
| | | | | total area, | monomeric | monomeric | of main peak |
| | | | | dimer peak | peak. | peak | |
| | | | | is 5% | | increases. | |
| 673 | scFab | TF | ND | stable | stable | stable | ND |
| | LL | (D3H44) | | monomeric, | monomeric, | monomeric, | |
| | (helical) | | | leading | leading | leading | |
| | | | | shoulder | shoulder | shoulder | |
| | | | | visible | visible | visible | |
| 665 | sc Fab | TF | 67.4 | Mainly | Mainly | Mainly | Mainly |
| | LCI | (D3H44) | | monomeric. | monomeric. | monomeric. | monomeric and |
| | | | | Dimer peak | Dimer | No | stable |
| | | | | visible at | peak | dimer | throughout the |
| | | | | 3.14 min. | disappears. | peak. | 7 days. |
| | | | | Main peak | Leading | Leading | Secondary |
| | | | | encompasses | shoulder | shoulder | peak visible; 5% |
| | | | | 95% of | on | on | of total area |
| | | | | total area, | monomeric | monomeric | of main peak |
| | | | | dimer peak | peak. | peak | |
| | | | | is 5% | | increases. | |

TABLE 10
SPR binding data. Data was acquired on fresh protein and after
storage for 1 week at room temperature. The stored samples
retain target binding to Her2 and tissue factor antigen.

| Variant | Fab type | Storage | CH-VL linker | KD | SD |
|---|---|---|---|---|---|
| V654 | 4D5 | −80 | a41 | 1.2E−09 | 2.E−10 |
| V654 | 4D5 | 7 d RT | a41 | 5.E−10 | 2.E−10 |
| V673 | D3H44 | −80 | a41 | 4.5E−11 | |
| V673 | D3H44 | 7 d RT | a41 | nd | |
| V695 | 4D5 | −80 | — | 6.E−10 | 1.E−10 |
| V695 | 4D5 | 7 d RT | — | 8.E−10 | 1.E−10 |
| V696 | D3H44 | −80 | — | 1.0E−10 | 6.E−11 |
| V696 | D3H44 | 7 d RT | — | 8.E−11 | 2.E−11 |
| V656 | 4D5 | −80 | GST18/GST26 | 2E−10 | 7.E−11 |
| V656 | 4D5 | 7 d RT | GST18/GST26 | 4.E−10 | 1.E−10 |
| V665 | D3H44 | −80 | GS20/GS24 | 6.2E−11 | 5.E−12 |
| V665 | D3H44 | 7 d RT | GS20/GS24 | 9.E−11 | 2.E−11 |
| V695 | 4D5 | −80 | — | 6.E−10 | 1.E−10 |
| V695 | 4D5 | 7 d RT | — | 8.E−10 | 1.E−10 |
| V696 | D3H44 | −80 | — | 1.0E−10 | 6.E−11 |
| V696 | D3H44 | 7 d RT | — | 8.E−11 | 2.E−11 |

The a41 linker identified in Table 10 corresponds to the Helical-41 linker noted in the legend to Table 3 and is also referred to elsewhere herein as H-41. Results indicated that all single chain Fab samples do not re-multimerize, at the relatively dilute concentration used, during the week-long study.

### Example 5

### Expression, Purification and Analysis of Bivalent Monospecific scMabs (Heterodimeric Fc) in CHO Cell Line. DNA Ratio of the Two Chains was 1:1 Chain A/Chain B (FIG. 11)

Bivalent monospecific scMabs described in table 11 were constructed. Bivalent monospecific scMabs were constructed using standard recombinant DNA cloning methods, using scFab designs described in Example 2. Briefly, each polypeptide of the bivalent scMabs were created by fusing the nucleic acid encoding the relevant scFab (either long linker or LCI design) to a nucleic acid encoding the Fc region of the antibody via a wild-type IgG1 linker. The Fc region used harbors mutations on the CH3 domains that allow formation of a heterodimeric antibody molecule (i.e. chain A has L351Y_F405A_Y407V mutations, and chain B the T366L_K392M_T394W mutations). Expression was performed in 2 mL HEK293 or CHO cultures. Cells were transfected in exponential growth phase (1.5 to 2 millions cells/ml) with PEI (Polyethylenimine linear 25 kDa dissolve in water to 1 mg/ml, Polysciences, cat#23966) and 1 ug DNA/ml of cells at a ratio PEI/DNA of 2.5:1. Salmon sperm DNA (70%) is added to complete 100 ug DNA. PEI is mixed to transfection medium in 1/20 volume of total transfection. The PEI/DNA mixture is vortexed and incubated at RT for 3 minutes. Transfection medium (pre-warmed at room temperature or 37° C.) is the same as that used for maintenance of cells (F17 media supplemented with 4 mM L-Glutamine, 0.1% Pluronic F68 and 0.025 mg/ml G418.).

For purification, the clarified culture medium was brought to room temperature and degassed before purification using a filter unit of 0.45 um. Single chain heterodimers and the WT IgG1 antibodies were purified by using protein A (Mabselect Sure). The column was equilibrated with 5 CV of PBS. The filtered medium was loaded on the Protein A column, which was subsequently washed with 10 CV of PBS. The antibodies were eluted with 10 CV citrate buffer pH 3.6 and antibody fractions collected. The pH was neutralized by adding 1/3 of the fraction volume of Tris buffer p H-11. Purified protein was desalted using a desalting column (Econo-Pac 10DG Columns from Bio-Rad). A representative SDS-PAGE gel is shown in FIG. 11.

TABLE 11

| | | scFab | | | IgG | | | Variant | |
|---|---|---|---|---|---|---|---|---|---|
| Source | Antigen | design | Linker 1 | Linker 2 | linker | Fc type | Fab type | number | KD (M) |
| 4D5 | HER2 | HET- | GS20 | GS24 | IgG1 | HET v613 | homodimer | 896 | 1.57E−10 |
| | | FC/Fab- | | | | (L351Y_F405A_Y407V/T366L_K392M_T394W) | | | |
| | | LCI | | | | | | | |
| 4D5 | HER2 | HET- | GS15 | GS24 | IgG1 | HET v613 | homodimer | 898 | 4.61E−10 |
| | | FC/Fab- | | | | (L351Y_F405A_Y407V/T366L_K392M_T394W) | | | |
| | | LCI | | | | | | | |
| 4D5 | HER2 | HET- | GS35 | | IgG1 | HET v613 | homodimer | 895 | 1.02E−10 |
| | | FC/Fab- | | | | (L351Y_F405A_Y407V/T366L_K392M_T394W) | | | |
| | | LL | | | | | | | |
| 4D5 | HER2 | HET- | GST32 | | IgG1 | HET v613 | homodimer | 894 | 5.55E−10 |
| | | FC/Fab- | | | | (L351Y_F405A_Y407V/T366L_K392M_T394W) | | | |
| | | LL | | | | | | | |
| 4D5 | HER2 | HET- | GST18 | GST26 | IgG1 | HET v613 | homodimer | 897 | 7.05E−10 |
| | | FC/Fab- | | | | (L351Y_F405A_Y407V/T366L_K392M_T394W) | | | |
| | | LCI | | | | | | | |
| D3H44 | TF | HET- | GS35 | | IgG1 | HET v613 | homodimer | 899 | 2.54E−25 |
| | | FC/Fab- | | | | (L351Y_F405A_Y407V/T366L_K392M_T394W) | | | |
| | | LL | | | | | | | |
| D3H44 | TF | HET- | helical41 | | IgG1 | HET v613 | homodimer | 900 | 5.61E−11 |
| | | FC/Fab- | | | | (L351Y_F405A_Y407V/T366L_K392M_T394W) | | | |
| | | LL | | | | | | | |
| D3H44 | TF | HET- | GS20 | GS24 | IgG1 | HET v613 | homodimer | 901 | 1.28E−12 |
| | | FC/Fab- | | | | (L351Y_F405A_Y407V/T366L_K392M_T394W) | | | |
| | | LCI | | | | | | | |
| D3H44 | TF | HET- | G20 | GS28 | IgG1 | HET v613 | homodimer | 902 | 1.89E−11 |
| | | FC/Fab- | | | | (L351Y_F405A_Y407V/T366L_K392M_T394W) | | | |
| | | LCI | | | | | | | |

### Example 6

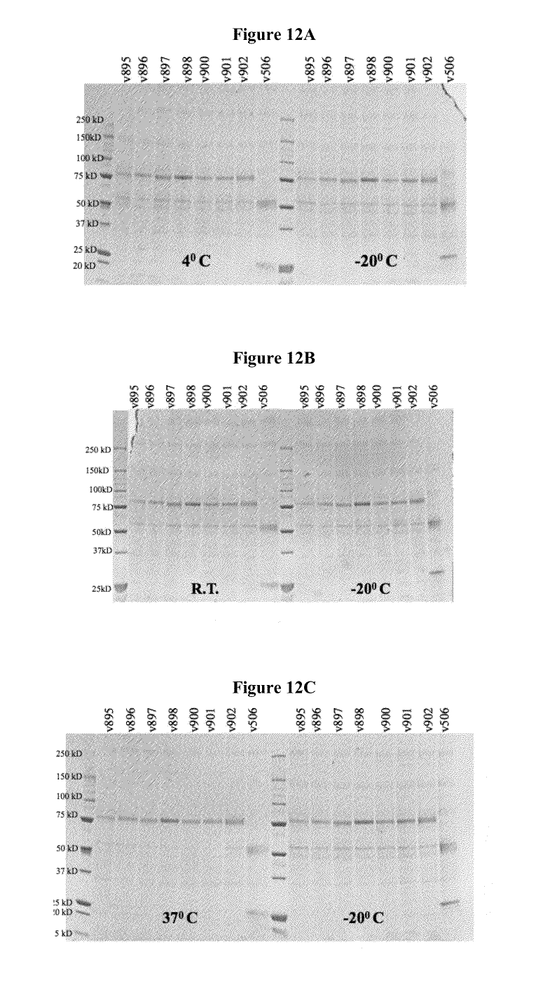

### Benchtop Stability Assay of scMab (FIG. 12A-12C)

The benchtop stability test (repeated in triplicate) consisted in taking a sample of each protein variant under analysis and monitoring breakdown and/or oligomerization over the course of 3 day storage at different temperatures and comparison to protein stored at −20 C. Assessment was done using reducing and non-reducing SDS-PAGE, loading 2.5 □g of protein per well. Protein concentration was determined by A280 nanodrop. Proteins were kept in vials at temperature of interest, with an initial sample taken at the beginning of the experiment (time 0). Subsequent samples were taken after 3 days. Every sample was denatured in Commassie Blue SDS buffer and stored at −80 C until final SDS-PAGE assessment.

Results indicate that all single chain Mab do not increase their multimeric state, at the relatively dilute concentration used, during the 3-day long study. IgG1 (Herceptin) was included as control. FIGS. 12A to 12C show the results of SDS-PAGE assessment. A summary of the results is shown in Table 12.

TABLE 12
Summary of benchtop stability testing of scMabs

| | | scFab | | | IgG | | Variant | Day 3 |
|---|---|---|---|---|---|---|---|---|
| Source | Antigen | design | Linker 1 | Linker 2 | linker | Fc type | number | 4C/RT/37C |
| 4D5 | HER2 | HET- | GS20 | GS24 | IgG1 | HET v613 | 896 | No significant |
| | | FC/Fab- | | | | (L351Y_F405A_Y407V/ | | degradation/ |
| | | LCI | | | | T366L_K392M_T394W) | | increase of |
| | | | | | | | | multimerization |
| | | | | | | | | visible |
| 4D5 | HER2 | HET- | GS15 | GS24 | IgG1 | HET v613 | 898 | No significant |
| | | FC/Fab- | | | | (L351Y_F405A_Y407V/ | | degradation/ |
| | | LCI | | | | T366L_K392M_T394W) | | increase of |
| | | | | | | | | multimerization |
| | | | | | | | | visible |
| 4D5 | HER2 | HET- | GS35 | | IgG1 | HET v613 | 895 | No significant |
| | | FC/Fab- | | | | (L351Y_F405A_Y407V/ | | degradation/ |
| | | LL | | | | T366L_K392M_T394W) | | increase of |
| | | | | | | | | multimerization |
| | | | | | | | | visible |
| 4D5 | HER2 | HET- | GST18 | GST26 | IgG1 | HET v613 | 897 | No significant |
| | | FC/Fab- | | | | (L351Y_F405A_Y407V/ | | degradation/ |
| | | LCI | | | | T366L_K392M_T394W) | | increase of |
| | | | | | | | | multimerization |
| | | | | | | | | visible |
| D3H44 | TF | HET- | helical41 | | IgG1 | HET v613 | 900 | No significant |
| | | FC/Fab- | | | | (L351Y_F405A_Y407V/ | | degradation/ |
| | | LL | | | | T366L_K392M_T394W) | | increase of |
| | | | | | | | | multimerization |
| | | | | | | | | visible |
| D3H44 | TF | HET- | GS20 | GS24 | IgG1 | HET v613 | 901 | No significant |
| | | FC/Fa | | | | (L351Y_F405A_Y407V/ | | degradation/ |
| | | b-LCI | | | | T366L_K392M_T394W) | | increase of |
| | | | | | | | | multimerization |
| | | | | | | | | visible |
| D3H44 | TF | HET- | G20 | GS28 | IgG1 | HET v613 | 902 | No significant |
| | | FC/Fab- | | | | (L351Y_F405A_Y407V/ | | degradation/ |
| | | LCI | | | | T366L_K392M_T394W) | | increase of |
| | | | | | | | | multimerization |
| | | | | | | | | visible |

No significant degradation of the product or increase of multimerization was visible in the course of the benchtop stability analysis.

### Example 7

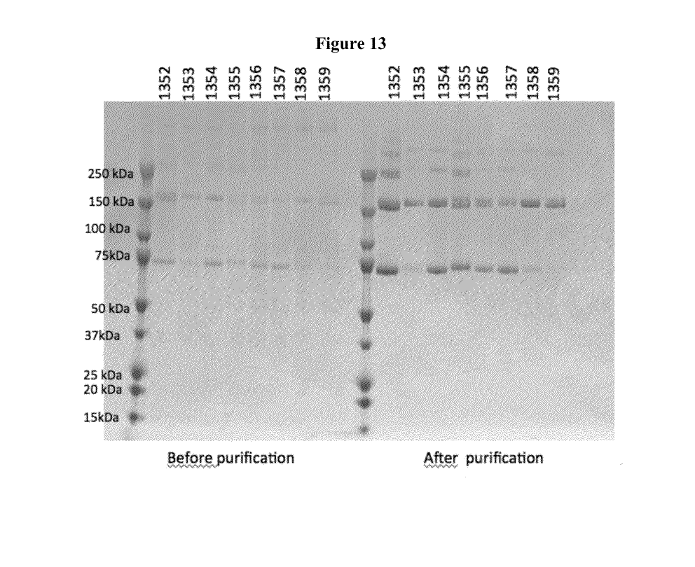

### Expression and Purification of Bivalent Bispecific scMabs (Heterodimeric Fc) in CHO Cell Line as Seen in FIG. 13. DNA Ratio of the Two Chains was 1:1 Chain A/Chain B

Bispecific Her2 (Trastuzumab)/TF (D3H44) scMabs as described in table 13 were constructed as follows. The approach used to create the bispecific scMab molecules was to combine heavy chains designed as described in Example 5 (ie harboring mutations on their CH3 domains that allow formation of a heterodimeric Ab molecule (ie, chain A has the L351Y_F405A_Y407V mutations, and chain B the T386L_K392M_T394W mutations). The mutations in the CH3 domains on the heavy chains of these bivalent scMabs allowed multiple combinations of bispecific scMabs that not only retain binding to two different antigens, but also possess Fab regions that harbor identical or different linker types (short or long linkers within the same long linker or LCI scaffold) and identical or different scaffolds (i.e. Long Linker vs LCI). The following groups of bi-specific scMabs were constructed: bi-specific molecules that have the same linker and same scaffold (i.e. v1353 and 1357); same scaffold but different linker (v1352, 1354, 1355, 1358); different scaffold, different linker (v1359, 1356). Expression was performed in 50 mL CHO cultures as described in Example 3.

For purification, the clarified culture medium was brought to room temperature and degassed before purification using a filter unit of 0.45 um. Single chain heterodimers and the WT IgG1 antibodies were purified by using protein A (Mabselect Sure). The column was equilibrated with 5 CV of PBS. The filtered medium was loaded on the Protein A column, which was subsequently washed with 10 CV of PBS. The antibodies were eluted with 10 CV citrate buffer pH 3.6 and antibody fractions collected. The pH was neutralized by adding 1/3 of the fraction volume of Tris buffer p H-11. Purified protein was desalted using a desalting column (Econo-Pac 10DG Columns from Bio-Rad). A summary of the affinity purification results is shown in Table 14.

TABLE 13

| | | Linker | Linker | | | |
|---|---|---|---|---|---|---|
| | | Format | Format | IgG | | Variant |
| Source/Antigen A | Source/Antigen B | Warhead A | Warhead B | linker | Fc type | number |
| 4D5/HER2 | D3H44/TF | GST32 | GS35 | IgG1 | HET v613 | 1352 |
| | | | | | (L351Y_F405AY407V/ | |
| | | | | | T366L_K392M_T394W) | |
| 4D5/HER2 | D3H44/TF | LCI 20/24 | LCI 20/24 | IgG1 | HET v613 | 1353 |
| | | | | | (L351Y_F405A_Y407V/ | |
| | | | | | T366L_K392M_T394W) | |
| 4D5/HER2 | D3H44/TF | LCI 18/26 | LCI 20/24 | IgG1 | HET v613 | 1354 |
| | | | | | (L351Y_F405A_Y407V/ | |
| | | | | | T366L_K392M_T394W) | |
| D3H44/TF | 4D5/HER2 | H41 | GS35 | IgG1 | HET v613 | 1355 |
| | | | | | (L351Y_F405A_Y407V/ | |
| | | | | | T366L_K392M_T394W) | |
| D3H44/TF | 4D5/HER2 | LCI 20/28 | GS35 | IgG1 | HET v613 | 1356 |
| | | | | | (L351Y_F405A_Y407V/ | |
| | | | | | T366L_K392M_T394W) | |
| 4D5/HER2 | D3H44/TF | GS35 | GS35 | IgG1 | HET v613 | 1357 |
| | | | | | (L351Y_F405AY407V/ | |
| | | | | | T366L_K392M_T394W) | |
| D3H44/TF | 4D5/HER2 | LCI 15/24 | LCI 20/24 | IgG1 | HET v613 | 1358 |
| | | | | | (L351Y_F405A_Y407V/ | |
| | | | | | T366L_K392M_T394W) | |
| 4D5/HER2 | D3H44/TF | LCI 15/24 | H41 | IgG1 | HET v613 | 1359 |
| | | | | | (L351Y_F405A_Y407V/ | |
| | | | | | T366L_K392M_T394W) | |

TABLE 14
Summary of purification of scMabs

| Variant | initial material | post prot A affinity |
|---|---|---|
| number | (mg) | (mg) |
| 1352 | 4.9 | 2.5 |
| 1353 | 1.25 | 0.46 |
| 1354 | 3.05 | 1.73 |
| 1355 | 2.65 | 1.55 |
| 1356 | 1.35 | 0.57 |
| 1357 | 1.75 | 0.88 |
| 1358 | 1.25 | 0.47 |
| 1359 | 1.6 | 0.5 |
| WT IgG | | 1-2 mg |
| (v506) | | (historical, CHO) |

### Example 8

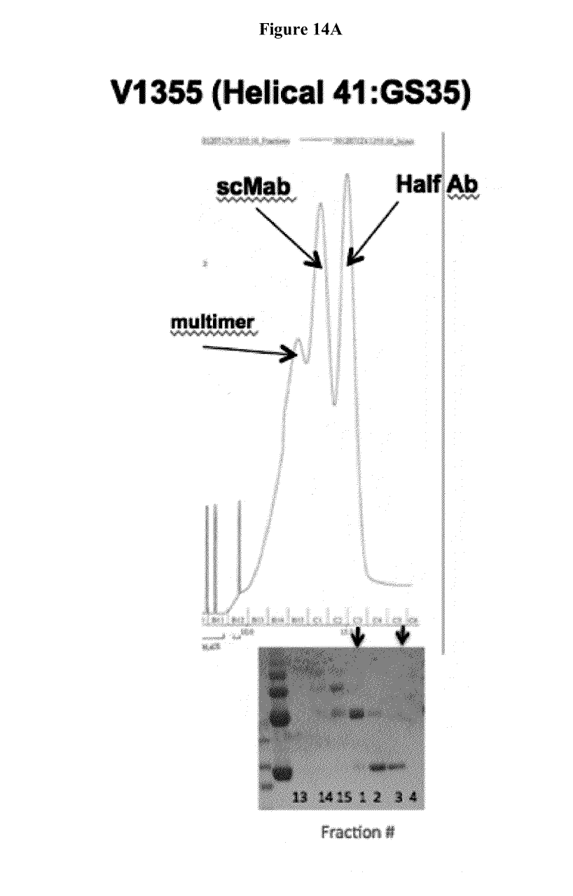

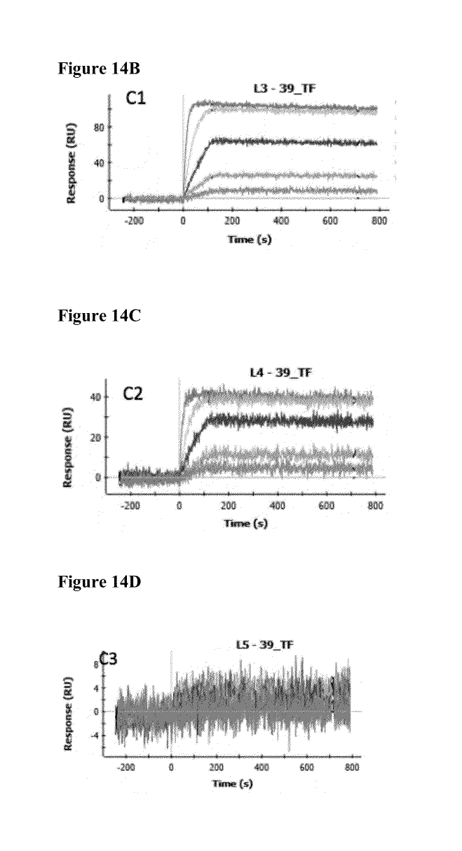

### SEC Profile and SPR Sandwich Assay of Bispecific scMab(LL/LL) (FIGS. 14, 15, and 16)

HER2 was immobilized on a GLM sensorchip and scMab was initially captured. TF binding was then determined by capturing TF on immobilized HER2-scMab. The dimeric bispecific scMab binds both HER2 and TF.

All surface plasmon resonance binding assays were carried out using a BioRad ProteOn XPR36 instrument (Bio-Rad Laboratories (Canada) Ltd. (Mississauga, ON)) with HBST running buffer (10 mM HEPES, 150 mM NaCl, 3.4 mM EDTA, and 0.05% Tween 20 pH 7.4) at a temperature of 25° C. The Her-2 capture surface was generated using a GLM sensorchip activated by a 1:5 dilution of the standard BioRad sNHS/EDC solutions injected for 300 s at 30 μL/min in the analyte (horizontal) direction. Immediately after the activation, a 4.0 μg/mL solution of Her-2 in 10 mM NaOAc pH 4.5 was injected in the ligand (vertical) direction at a flow rate of 25 μL/min until approximately 3000 resonance units (RUs) were immobilized. Remaining active groups were quenched by a 300 s injection of 1M ethanolamine at 30 μL/min in the analyte direction, and this also ensures mock-activated interspots are created for blank referencing. One to five single chain Mab variants were simultaneously injected in individual ligand channels for 240 s at flow 25 μL/min. This resulted in a saturating capture onto the Her-2 surface. Following antibody capture, TF analytes were passed over the chip, bound by the bispecific single chain molecules. Sensorgrams were aligned and double-referenced using the buffer blank injection and interspots, and the resulting sensorgrams were analyzed using ProteOn Manager software v3.0.

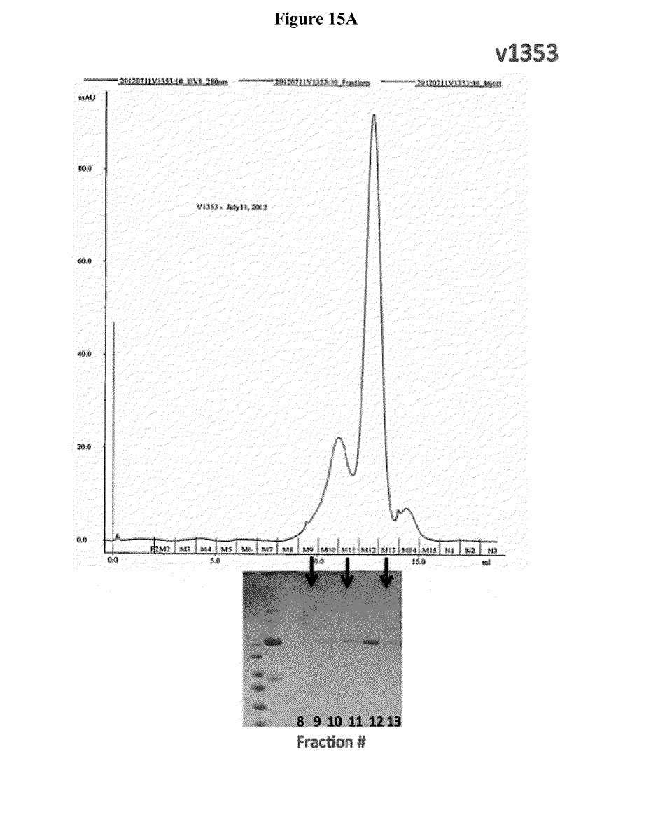

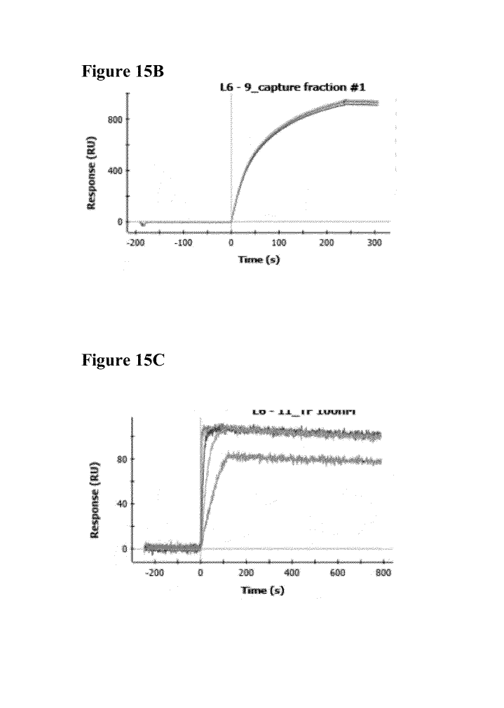

As seen in FIGS. 15A-15C, SEC profile and SPR sandwich assay of bispecific scMab (LCI/LCI) was performed. HER2 was immobilized on chip and scMab was initially captured. Tissue factor (TF) binding was then determined by capturing TF on immobilized HER2-scMab. The dimeric bispecific scMab binds both HER2 and TF. The level of monomeric species was significantly less than that observed with the long linker. A standard protocol for size exclusion chromatography was used, employing a Superdex S200 (16/60) and following manufacturer instructions. Running buffer was PBS.

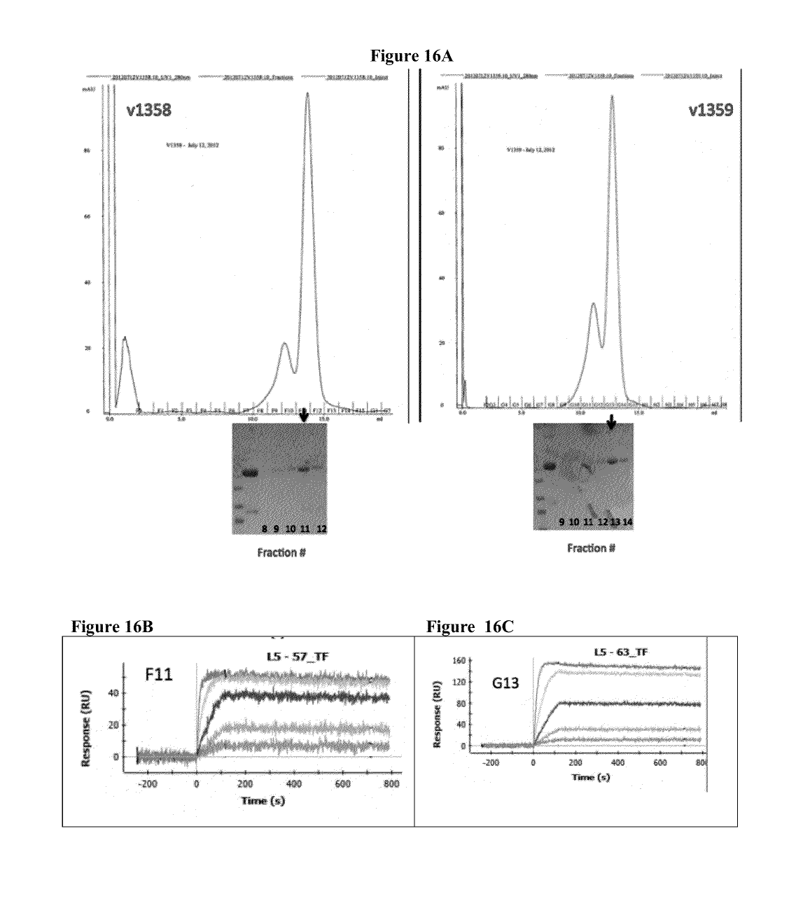

As shown in FIGS. 16A-16C, SEC and target binding profile of bispecific scMab's (LCI/LCI:1358; LCI/LL: 1359) was performed. HER2 was immobilized on chip and scMab was initially captured. TF binding was then determined by capturing TF on immobilized HER2-scMab. The dimeric bispecific scMab binds both HER2 and TF. A standard protocol for size exclusion chromatography was used, employing a Superdex S200 (16/60) and following manufacturer instructions. Running buffer was PBS. Table 15 provides the K_{D} for the variants tested.

TABLE 15
Affinity of variants for TF and HER2. Values reported here
are averages of SPR measurements performed with different
directionality (ie for TF binding, flowing TF antigen over
Her2 captured scMabs, flowing TF antigen over IgG captured
scMabs and flowing TF antigen over immobilized scMabs; for
Her2 binding, flowing Her2 antigen over IgG captured scMabs
and flowing Her2 antigen over immobilized scMabs).

| Variant | K_{D} for TF | K_{D} for HER2 |
|---|---|---|
| 1353 | 2.01E−11 | 3.22E−10 |
| 1355 | 2.3E−11 | 5.52E−10 |
| 1359 | 2.65E−11 | 2.01E−11 |
| Control 696 | 5.15E−11 | |
| Control 695 | | 8.37E−10 |
| Control 506 | | 5.27E−10 |

Description of control variants: variant 506 is a control antibody that binds HER2, based on the sequence of Herceptin™.

Table 16 provides data indicating the ability of various bi-specific scMab to bind to HER2 or tissue factor. Binding was measured using the sandwich SPR binding assay described in this example.

TABLE 16
Bispecific scMab with the 4D5 and D3H44 Mabs (LL/LL, LCI/LCI
and LL/LCI format), bispecific binding is observed to the
target antigen Her2 and Tissue factor. Variant scMabs 1352
and 1357 with Long Linkers on both arms cannot bind TF.

| | Observed Binding |
|---|---|
| Variant | for dimer scMab |
| 1352 | HER2 |
| 1355 | HER2/TF |
| 1357 | HER2 |
| 1353 | HER2/TF |
| 1354 | HER2/TF |
| 1358 | HER2/TF |
| 1359 | HER2/TF |
| 1356 | HER2/TF |

### Example 9

### Expression, Purification and Testing of Bivalent Bispecific scMabs (CD3/CD19)

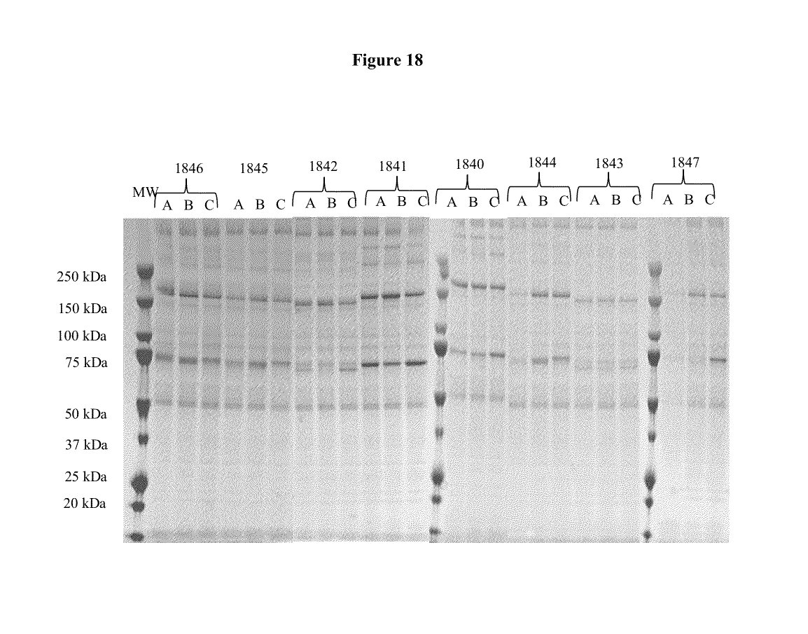

Bivalent, bi-specific scMabs were designed to bind to CD3 and CD19, and a description of these constructs is found in Table 17. These constructs were prepared using standard recombinant DNA methods. The constructs were prepared by breaking up the sequences of the Fab of anti-CD3 teplizumab and of anti-CD19 MOR208 into their VH, VL, CH and CL components. For LCI constructs, the VH of the Fab was connected by a first linker to the light chain (composed of VL and CL) and a second linker connected the light chain to the CH of the Fab. The Fab was then connected to an IGG1 hinge+Fc bearing the mutations T350V_L351Y_F405A_Y407V on chain A, and T350V_T366L_K392L_T394W on chain B. For LL constructs, the full light chain (composed of VL and CL) was connected with a linker to the heavy chain of the Fab which was then connected to an IGG1 hinge+Fc bearing the mutations T350V_L351Y_F405A_Y407V on chain A, and T350V_T366L_K392L_T394W on chain B. The sequences of the anti-CD3 (teplizumab) and anti-CD19 (MOR208) Fabs used in variants 1840 to 1847 were obtained from the literature (Tabs database, a service provided by Craic Computing LLC). The CD19 binding scFv used in 1853 was derived from the sequence of blinatumomab. The CD3 binding scFv sequence in variant 1861 was derived from muronomab while the variant 1862 sequence was derived from blinatumomab. These variants were expressed and purified as also described in Example 3. An exemplary SDS-PAGE gel showing the expression of these variants is shown in FIG. 18.

These variants were tested for their ability to bind to cells expressing the target antigen using a live cell ELISA binding assay using filter plates and wash steps carried out using a vacuum manifold (method developed by Anne Marcil at the National Research Council, Montreal, Canada). The method was carried out as follows. Cells were maintained in exponential growth phase and then washed, counted and distributed at 10E5 cells per well in 50% medium, 50% blocking buffer. Dilutions of variants in blocking buffer were added to the cells and incubated for 1 hour at 4° C. After collection and four washes in medium, an HRP-conjugated goat anti-human IgG was added to cells which were incubated for 1 hour at 4° C. After collection and four washes in medium and 3 washes in PBS, TMB substrate was added to cells which were incubated for 25 minutes at room temperature. The reaction was stopped by addition of H₂SO4 and OD read at 450 nm. The ELISA results are depicted in Tables 17 and 18. A, B, and C refer to the ratio of Chain A to Chain B used in expression of the variant: Ratio A=Chain A/Chain B=1:1 A/B=50%/50%; Ratio B=Chain A/Chain B=2:1 A/B=66%/34%; Ratio C=Chain A/Chain B=1:2 A/B=34%/66%.

TABLE 17
Summary of binding data

| | | Protein-A | ELISA results | |
|---|---|---|---|---|
| CD3xCD19 | | Concentration | HBP-ALL | RAJI |
| ID | Name* | (μg/ml | (CD3+) | (CD19+) |
| 1840 A | scMAB_LCI_CD3xCD19_GS20- | 62 | ++ | +++ |
| | GS24_GS20-GS24 | | | |
| 1841 B | scMAB_LCI_CD3xCD19_GS15- | 65 | ++ | +++ |
| | GS24_GS15-GS24 | | | |
| 1842 B | scMAB_LCI_CD3xCD19_GS20- | 33 | ++ | +++ |
| | GS24_GS20-GS24_SS | | | |
| 1843 B | scMAB_LCI_CD3xCD19_GS15- | 31 | ++ | +++ |
| | GS24_GS15-GS24_SS | | | |
| 1844 B | scMAB_LL_CD3xCD19_HH41_HH41 | 59 | ++ | +++ |
| 1844 C | scMAB_LL_CD3xCD19_HH41_HH41 | 57 | + | +++ |
| 1845 A | scMAB_LL_CD3xCD19_GSE34_GSE34 | 25 | ++ | + |
| | | | | + |
| 1845 B | scMAB_LL_CD3xCD19_GSE34_GSE34 | 41 | ++ | +++ |
| 1845 C | scMAB_LL_CD3xCD19_GSE34_GSE34 | 35 | + | +++ |
| 1846 A | scMAB_LL_CD3xCD19_HH41_GSE34 | 43 | ++ | +++ |
| 1846 B | scMAB_LL_CD3xCD19_HH41_GSE34 | 48 | ++ | +++ |
| 1846 C | scMAB_LL_CD3xCD19_HH41_GSE34 | 41 | + | +++ |
| 1847 B | scMAB_LL_CD3xCD19_VH-VL- | 40 | + | +++ |
| | HH41_HH41 | | | |
| 1853 B | FAB_CD3_scFv_CD19 | 59 | ++ | +++ |
| 1861 C | FAB_CD19_scFv_CD3_873 | 26 | ++ | +++ |
| 1862 C | FAB_CD19_scFv_CD3_875 | 33 | + | +++ |

*The variant names are generated as desribed for a) scMAB_LCI_CD3xCD19_GS20-GS24_GS20-GS24 - single chain scMab, LCI design, CD3 Fab with GS20 linker between V_{H}-V_{L} and a GS24 linker between C_{L}-C_{H} on chain A. CD19 Fab with with GS20 linker between VH-V_{L} , and a GS24 linker between C_{L}-CH on chain B; and b) scMAB_LL_CD3xCD19_HH41_HH41 - single chain Mab, long linker design, CD3 Fab with HH41 linker between the light and heavy chain of chain A; and a CD19 Fab with HH41 linker between the light chain and the heavy chain of chain B.

TABLE 18
Binding data for controls

| ID | Name | | | |
|---|---|---|---|---|
| Controls | | | | |
| 2176 A | MOR208-CD19 FSA | 62 | − | +++ |
| OAA and FSA controls | | | | |
| 2177 A | teplizumab-hOKT3-CD3 FSA | 56 | +++ | +/− |
| Negative control | | | | |
| 1041 A | MET_for_MA_v857_OAA | 103 | − | − |
| | (HER-B /Fc-A) | | | |
| Positive control | | | | |
| 875 | Fc-scFv-bispecific:bispecific | 19 | + | ++ |
| (40:60) | CD19-CD3(VL-VH-OKT3) | | | |

Legend for ELISA results in Tables 16 and 17
+++: OD450 > 2.0 at 1/60 dilution
++: OD450 between 1.0 and 2.0 at 1/60 dilution
+: OD450 < 1.0 at 1/60 dilution
+/−: OD450 below 0.100 at 1/60 dilution, but higher dilutions positive (higher than neg control)
−: OD450 = or < negative control 1041

All variants contained the anti-CD3 warhead from teplizumab, which showed little to no binding to the Raji CD19-containing cells (Table 16). All variants had the anti-CD19 warhead from MOR208, which showed no binding to HBP-All CD3-containing cells (Table 16). The negative control contained the same Fc as the variants and showed no binding to either antigen (Table 17). Therefore, variants that show binding to both antigens are bispecific due to the independent binding of each of their warheads. Table 16 shows that all variants are bispecific with two functioning warheads.

### Example 10

### Summary of Sequences Provided

The table below provides the SEQ ID NOs: for the amino acid sequences that make up the variants listed therein. All amino acid sequences include the sequence EFATMAVMAPRTLVLLLSGALALTQTWAG (which includes the signal peptide sequence and residues generated in the restriction site used for cloning) and the stop codon is marked with an asterisk.

| | | | SEQ ID NO: |
|---|---|---|---|
| | | SEQ ID NO: | (second chain, |
| Variant | Format | (first chain) | if present) |
| 673 | scFab | 3 | — |
| 654 | scFab | 1 | — |
| 900 | scMab | 9 | 5 |
| 1355 | scMab | 9 | 25 |
| 1359 | scMab | 24 | 5 |
| 1844 | scMab | 12 | 13 |
| 1846 | scMab | 12 | 4 |
| 665 | scFab | 14 | — |
| 656 | scFab | 2 | — |
| 896 | scMab | 21 | 23 |
| 897 | scMab | 26 | 7 |
| 898 | scMab | 24 | 22 |
| 901 | scMab | 6 | 10 |
| 902 | scMab | 11 | 8 |
| 1353 | scMab | 21 | 10 |
| 1354 | scMab | 26 | 10 |
| 1356 | scMab | 11 | 25 |
| 1358 | scMab | 22 | 6 |
| 1841 | scMab | 15 | 16 |
| 1842 | scMab | 17 | 18 |
| 1843 | scMab | 19 | 20 |

It is understood that the examples and embodiments described herein are for illustrative purposes only and that various modifications or changes in light thereof will be suggested to persons skilled in the art and are to be included within the spirit and purview of this application and scope of the appended claims.

SEQ ID NO: 1
EFATMAVMAPRTLVLLLSGALALTQTWAGDIQMTQSPSSLSASVGDRVTITCRASQDVNTAVAWYQQKPGKAPKLLIYSA
SFLYSGVPSRFSGSRSGTDFTLTISSLQPEDFATYYCQQHYTTPPTFGQGTKVEIKRTVAAPSVFIFPPSDEQLKSGTAS
VVCLLNNFYPREAKVQWKVDNALQSGNSQESVTEQDSKDSTYSLSSTLTLSKADYEKHKVYACEVTHQGLSSPVTKSFNR
GECGGSGGGSGSSADDAKKDAAKKDDAKKDDAKKDGGGSGGGSGEVQLVESGGGLVQPGGSLRLSCAASGFNIKDTYIHW
VRQAPGKGLEWVARIYPTNGYTRYADSVKGRFTISADTSKNTAYLQMNSLRAEDTAVYYCSRWGGDGFYAMDYWGQGTLV
TVSSASTKGPSVFPLAPSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHT*GS
SEQ ID NO: 2
EFATMAVMAPRTLVLLLSGALALTQTWAGEVQLVESGGGLVQPGGSLRLSCAASGFNIKDTYIHWVRQAPGKGLEWVARI
YPTNGYTRYADSVKGRFTISADTSKNTAYLQMNSLRAEDTAVYYCSRWGGDGFYAMDYWGQGTLVTVSSGSTSGSGKPGS
GEGSTKGDIQMTQSPSSLSASVGDRVTITCRASQDVNTAVAWYQQKPGKAPKLLIYSASFLYSGVPSRFSGSRSGTDFTL
TISSLQPEDFATYYCQQHYTTPPTFGQGTKVEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSPVTKSFNRGECGSTSGSTSGSGKPGSGEGS
TKGGSTSSTKGPSVFPLAPSSKSTSGGTAALGCLVKDYFPEPVTVSWNSGALTSGVHTFPAVLQSSGLYSLSSVVTVPSS
SLGTQTYICNVNHKPSNTKVDKKVEPKSCDKTHT*GS
SEQ ID NO: 3
EFATMAVMAPRTLVLLLSGALALTQTWAGDIQMTQSPSSLSASVGDRVTITCRASRDIKSYLNWYQQKPGKAPKVLIYYA
TSLAEGVPSRFSGSGSGTDYTLTISSLQPEDFATYYCLQHGESPWTFGQGTKVEIKRTVAAPSVFIFPPSDEQLKSGTAS
VVCLLNNFYPREAKVQWKVDNALQSGNSQESVTEQDSKDSTYSLSSTLTLSKADYEKHKVYACEVTHQGLSSPVTKSFNR
GECGGSGGGSGSSADDAKKDAAKKDDAKKDDAKKDGGGSGGGSGEVQLVESGGGLVQPGGSLRLSCAASGFNIKEYYMHW
VRQAPGKGLEWVGLIDPEQGNTIYDPKFQDRATISADNSKNTAYLQMNSLRAEDTAVYYCARDTAAYFDYWGQGTLVTVS
SASTKGPSVFPLAPSSKSTSGGTAALGCLVKDYFPEPVTVSWNSGALTSGVHTFPAVLQSSGLYSLSSVVTVPSSSLGTQ
TYICNVNHKPSNTKVDKKVEPKSCDKTHT*GS
SEQ ID NO: 4
EFATMAVMAPRTLVLLLSGALALTQTWAGDIVMTQSPATLSLSPGERATLSCRSSKSLQNVNGNTYLYWFQQKPGQSPQL
LIYRMSNLNSGVPDRFSGSGSGTEFTLTISSLEPEDFAVYYCMQHLEYPITFGAGTKLEIKRTVAAPSVFIFPPSDEQLK
SGTASVVCLLNNFYPREAKVQWKVDNALQSGNSQESVTEQDSKDSTYSLSSTLTLSKADYEKHKVYACEVTHQGLSSPVT
KSFNRGECSGGGSGGGSEGGGSEGGGSEGGGSEGGGSGGGSGEVQLVESGGGLVKPGGSLKLSCAASGYTFTSYVMHWVR
QAPGKGLEWIGYINPYNDGTKYNEKFQGRVTISSDKSISTAYMELSSLRSEDTAMYYCARGTYYYGTRVFDYWGQGTLVT
VSSASTKGPSVFPLAPSSKSTSGGTAALGCLVKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKVSNKALPAPIEKTISKAKGQPREPQVYVLPPS
RDELTKNQVSLLCLVKGFYPSDIAVEWESNGQPENNYLTWPPVLDSDGSFFLYSKLTVDKSRWQQGNVFSCSVMHEALHN
HYTQKSLSLSPGK*GS
SEQ ID NO: 5
EFATMAVMAPRTLVLLLSGALALTQTWAGDIQMTQSPSSLSASVGDRVTITCRASRDIKSYLNWYQQKPGKAPKVLIYYA
TSLAEGVPSRFSGSGSGTDYTLTISSLQPEDFATYYCLQHGESPWTFGQGTKVEIKRTVAAPSVFIFPPSDEQLKSGTAS
VVCLLNNFYPREAKVQWKVDNALQSGNSQESVTEQDSKDSTYSLSSTLTLSKADYEKHKVYACEVTHQGLSSPVTKSFNR
GECGGSGGGSGSSADDAKKDAAKKDDAKKDDAKKDGGGSGGGSGEVQLVESGGGLVQPGGSLRLSCAASGFNIKEYYMHW
VRQAPGKGLEWVGLIDPEQGNTIYDPKFQDRATISADNSKNTAYLQMNSLRAEDTAVYYCARDTAAYFDYWGQGTLVTVS
SASTKGPSVFPLAPSSKSTSGGTAALGCLVKDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKALPAPIEKTISKAKGQPREPQVYTLPPSRD
ELTKNQVSLLCLVKGFYPSDIAVEWESNGQPENNYMTWPPVLDSDGSFFLYSKLTVDKSRWQQGNVFSCSVMHEALHNHY
TQKSLSLSPGK*GS
SEQ ID NO: 6
EFATMAVMAPRTLVLLLSGALALTQTWAGEVQLVESGGGLVQPGGSLRLSCAASGFNIKEYYMHWVRQAPGKGLEWVGLI
DPEQGNTIYDPKFQDRATISADNSKNTAYLQMNSLRAEDTAVYYCARDTAAYFDYWGQGTLVTVSSGGGGSGGGGSGGGG
SGGGGSDIQMTQSPSSLSASVGDRVTITCRASRDIKSYLNWYQQKPGKAPKVLIYYATSLAEGVPSRFSGSGSGTDYTLT
ISSLQPEDFATYYCLQHGESPWTFGQGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGECGGGGSGGGGSGGGGSGGGGS
GGGGSTKGPSVFPLAPSSKSTSGGTAALGCLVKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKVSNKALPAPIEKTISKAKGQPREPQVYTYPPS
RDELTKNQVSLTCLVKGFYPSDIAVEWESNGQPENNYKTTPPVLDSDGSFALVSKLTVDKSRWQQGNVFSCSVMHEALHN
HYTQKSLSLSPGK*GS
SEQ ID NO: 7
EFATMAVMAPRTLVLLLSGALALTQTWAGEVQLVESGGGLVQPGGSLRLSCAASGFNIKDTYIHWVRQAPGKGLEWVARI
YPTNGYTRYADSVKGRFTISADTSKNTAYLQMNSLRAEDTAVYYCSRWGGDGFYAMDYWGQGTLVTVSSGSTSGSGKPGS
GEGSTKGDIQMTQSPSSLSASVGDRVTITCRASQDVNTAVAWYQQKPGKAPKLLIYSASFLYSGVPSRFSGSRSGTDFTL
TISSLQPEDFATYYCQQHYTTPPTFGQGTKVEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSPVTKSFNRGECGSTSGSTSGSGKPGSGEGS
TKGGSTSSTKGPSVFPLAPSSKSTSGGTAALGCLVKDYFPEPVTVSWNSGALTSGVHTFPAVLQSSGLYSLSSVVTVPSS
SLGTQTYICNVNHKPSNTKVDKKVEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISKAKGQPREPQVYTL
PPSRDELTKNQVSLLCLVKGFYPSDIAVEWESNGQPENNYMTWPPVLDSDGSFFLYSKLTVDKSRWQQGNVFSCSVMHEA
LHNHYTQKSLSLSPGK*GS
SEQ ID NO: 8
EFATMAVMAPRTLVLLLSGALALTQTWAGEVQLVESGGGLVQPGGSLRLSCAASGFNIKEYYMHWVRQAPGKGLEWVGLI
DPEQGNTIYDPKFQDRATISADNSKNTAYLQMNSLRAEDTAVYYCARDTAAYFDYWGQGTLVTVSSGGGGSGGGGSGGGG
SGGGGSDIQMTQSPSSLSASVGDRVTITCRASRDIKSYLNWYQQKPGKAPKVLIYYATSLAEGVPSRFSGSGSGTDYTLT
ISSLQPEDFATYYCLQHGESPWTFGQGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGECGGGGSGGGGSGGGGSGGGGS
GGGGSGGGSTKGPSVFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKCKVSNKALPAPIEKTISKAKGQPREPQVYT
LPPSRDELTKNQVSLLCLVKGFYPSDIAVEWESNGQPENNYMTWPPVLDSDGSFFLYSKLTVDKSRWQQGNVFSCSVMHE
ALHNHYTQKSLSLSPGK*GS
SEQ ID NO: 9
EFATMAVMAPRTLVLLLSGALALTQTWAGDIQMTQSPSSLSASVGDRVTITCRASRDIKSYLNWYQQKPGKAPKVLIYYA
TSLAEGVPSRFSGSGSGTDYTLTISSLQPEDFATYYCLQHGESPWTFGQGTKVEIKRTVAAPSVFIFPPSDEQLKSGTAS
VVCLLNNFYPREAKVQWKVDNALQSGNSQESVTEQDSKDSTYSLSSTLTLSKADYEKHKVYACEVTHQGLSSPVTKSFNR
GECGGSGGGSGSSADDAKKDAAKKDDAKKDDAKKDGGGSGGGSGEVQLVESGGGLVQPGGSLRLSCAASGFNIKEYYMHW
VRQAPGKGLEWVGLIDPEQGNTIYDPKFQDRATISADNSKNTAYLQMNSLRAEDTAVYYCARDTAAYFDYWGQGTLVTVS
SASTKGPSVFPLAPSSKSTSGGTAALGCLVKDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKALPAPIEKTISKAKGQPREPQVYTYPPSRD
ELTKNQVSLTCLVKGFYPSDIAVEWESNGQPENNYKTTPPVLDSDGSFALVSKLTVDKSRWQQGNVFSCSVMHEALHNHY
TQKSLSLSPGK*GS
SEQ ID NO: 10
EFATMAVMAPRTLVLLLSGALALTQTWAGEVQLVESGGGLVQPGGSLRLSCAASGFNIKEYYMHWVRQAPGKGLEWVGLI
DPEQGNTIYDPKFQDRATISADNSKNTAYLQMNSLRAEDTAVYYCARDTAAYFDYWGQGTLVTVSSGGGGSGGGGSGGGG
SGGGGSDIQMTQSPSSLSASVGDRVTITCRASRDIKSYLNWYQQKPGKAPKVLIYYATSLAEGVPSRFSGSGSGTDYTLT
ISSLQPEDFATYYCLQHGESPWTFGQGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGECGGGGSGGGGSGGGGSGGGGS
GGGGSTKGPSVFPLAPSSKSTSGGTAALGCLVKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKVSNKALPAPIEKTISKAKGQPREPQVYTLPPS
RDELTKNQVSLLCLVKGFYPSDIAVEWESNGQPENNYMTWPPVLDSDGSFFLYSKLTVDKSRWQQGNVFSCSVMHEALHN
HYTQKSLSLSPGK*GS
SEQ ID NO: 11
EFATMAVMAPRTLVLLLSGALALTQTWAGEVQLVESGGGLVQPGGSLRLSCAASGFNIKEYYMHWVRQAPGKGLEWVGLI
DPEQGNTIYDPKFQDRATISADNSKNTAYLQMNSLRAEDTAVYYCARDTAAYFDYWGQGTLVTVSSGGGGSGGGGSGGGG
SGGGGSDIQMTQSPSSLSASVGDRVTITCRASRDIKSYLNWYQQKPGKAPKVLIYYATSLAEGVPSRFSGSGSGTDYTLT
ISSLQPEDFATYYCLQHGESPWTFGQGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGECGGGGSGGGGSGGGGSGGGGS
GGGGSGGGSTKGPSVFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKCKVSNKALPAPIEKTISKAKGQPREPQVYT
YPPSRDELTKNQVSLTCLVKGFYPSDIAVEWESNGQPENNYKTTPPVLDSDGSFALVSKLTVDKSRWQQGNVFSCSVMHE
ALHNHYTQKSLSLSPGK*GS
SEQ ID NO: 12
EFATMAVMAPRTLVLLLSGALALTQTWAGDIQMTQSPSSLSASVGDRVTITCSASSSVSYMNWYQQTPGKAPKRWIYDTS
KLASGVPSRFSGSGSGTDYTFTISSLQPEDIATYYCQQWSSNPFTFGQGTKLQITRTVAAPSVFIFPPSDEQLKSGTASV
VCLLNNFYPREAKVQWKVDNALQSGNSQESVTEQDSKDSTYSLSSTLTLSKADYEKHKVYACEVTHQGLSSPVTKSFNRG
ECGGSGGGSGSSADDAKKDAAKKDDAKKDDAKKDGGGSGGGSGQVQLVQSGGGVVQPGRSLRLSCKASGYTFTRYTMHWV
RQAPGKGLEWIGYINPSRGYTNYNQKVKDRFTISRDNSKNTAFLQMDSLRPEDTGVYFCARYYDDHYCLDYWGQGTPVTV
SSASTKGPSVFPLAPSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPIEKTISKAKGQPREPQVYVYPPSR
DELTKNQVSLTCLVKGFYPSDIAVEWESNGQPENNYKTTPPVLDSDGSFALVSKLTVDKSRWQQGNVFSCSVMHEALHNH
YTQKSLSLSPGK*GS
SEQ ID NO: 13
EFATMAVMAPRTLVLLLSGALALTQTWAGDIVMTQSPATLSLSPGERATLSCRSSKSLQNVNGNTYLYWFQQKPGQSPQL
LIYRMSNLNSGVPDRFSGSGSGTEFTLTISSLEPEDFAVYYCMQHLEYPITFGAGTKLEIKRTVAAPSVFIFPPSDEQLK
SGTASVVCLLNNFYPREAKVQWKVDNALQSGNSQESVTEQDSKDSTYSLSSTLTLSKADYEKHKVYACEVTHQGLSSPVT
KSFNRGECGGSGGGSGSSADDAKKDAAKKDDAKKDDAKKDGGGSGGGSGEVQLVESGGGLVKPGGSLKLSCAASGYTFTS
YVMHWVRQAPGKGLEWIGYINPYNDGTKYNEKFQGRVTISSDKSISTAYMELSSLRSEDTAMYYCARGTYYYGTRVFDYW
GQGTLVTVSSASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGKEYKCKVSNKALPAPIEKTISKAKGQPREPQ
VYVLPPSRDELTKNQVSLLCLVKGFYPSDIAVEWESNGQPENNYLTWPPVLDSDGSFFLYSKLTVDKSRWQQGNVFSCSV
MHEALHNHYTQKSLSLSPGK*GS
SEQ ID NO: 14
EFATMAVMAPRTLVLLLSGALALTQTWAGEVQLVESGGGLVQPGGSLRLSCAASGFNIKEYYMHWVRQAPGKGLEWVGLI
DPEQGNTIYDPKFQDRATISADNSKNTAYLQMNSLRAEDTAVYYCARDTAAYFDYWGQGTLVTVSSGGGGSGGGGSGGGG
SGGGGSDIQMTQSPSSLSASVGDRVTITCRASRDIKSYLNWYQQKPGKAPKVLIYYATSLAEGVPSRFSGSGSGTDYTLT
ISSLQPEDFATYYCLQHGESPWTFGQGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGECGGGGSGGGGSGGGGSGGGGS
GGGGSTKGPSVFPLAPSSKSTSGGTAALGCLVKDYFPEPVTVSWNSGALTSGVHTFPAVLQSSGLYSLSSVVTVPSSSLG
TQTYICNVNHKPSNTKVDKKVEPKSCDKTHT*GS
SEQ ID NO: 15
EFATMAVMAPRTLVLLLSGALALTQTWAGQVQLVQSGGGVVQPGRSLRLSCKASGYTFTRYTMHWVRQAPGKGLEWIGYI
NPSRGYTNYNQKVKDRFTISRDNSKNTAFLQMDSLRPEDTGVYFCARYYDDHYCLDYWGQGTPVTVSSGGGGSGGGGSGG
GGSDIQMTQSPSSLSASVGDRVTITCSASSSVSYMNWYQQTPGKAPKRWIYDTSKLASGVPSRFSGSGSGTDYTFTISSL
QPEDIATYYCQQWSSNPFTFGQGTKLQITRTVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKSFNRGECGGGGSGGGGSGGGGSGGGGSGGGG
STKGPSVFPLAPSSKSTSGGTAALGCLVKDYFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYVYPPSRDEL
TKNQVSLTCLVKGFYPSDIAVEWESNGQPENNYKTTPPVLDSDGSFALVSKLTVDKSRWQQGNVFSCSVMHEALHNHYTQ
KSLSLSPGK*GS
SEQ ID NO: 16
EFATMAVMAPRTLVLLLSGALALTQTWAGEVQLVESGGGLVKPGGSLKLSCAASGYTFTSYVMHWVRQAPGKGLEWIGYI
NPYNDGTKYNEKFQGRVTISSDKSISTAYMELSSLRSEDTAMYYCARGTYYYGTRVFDYWGQGTLVTVSSGGGGSGGGGS
GGGGSDIVMTQSPATLSLSPGERATLSCRSSKSLQNVNGNTYLYWFQQKPGQSPQLLIYRMSNLNSGVPDRFSGSGSGTE
FTLTISSLEPEDFAVYYCMQHLEYPITFGAGTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSFNRGECGGGGSGGGGSGGGGSG
GGGSGGGGSTKGPSVFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKCKVSNKALPAPIEKTISKAKGQPREPQVYV
LPPSRDELTKNQVSLLCLVKGFYPSDIAVEWESNGQPENNYLTWPPVLDSDGSFFLYSKLTVDKSRWQQGNVFSCSVMHE
ALHNHYTQKSLSLSPGK*GS
SEQ ID NO: 17
EFATMAVMAPRTLVLLLSGALALTQTWAGQVQLVQSGGGVVQPGRSLRLSCKASGYTFTRYTMHWVRQAPGKCLEWIGYI
NPSRGYTNYNQKVKDRFTISRDNSKNTAFLQMDSLRPEDTGVYFCARYYDDHYCLDYWGQGTPVTVSSGGGGSGGGGSGG
GGSGGGGSDIQMTQSPSSLSASVGDRVTITCSASSSVSYMNWYQQTPGKAPKRWIYDTSKLASGVPSRFSGSGSGTDYTF
TISSLQPEDIATYYCQQWSSNPFTFGCGTKLQITRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSPVTKSFNRGECGGGGSGGGGSGGGGSGGGG
SGGGGSTKGPSVFPLAPSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFLFPPKPKDTLMISRTPEVTCVVVDVSHEDPEV
KFNWYVDGVEVHNAKTKPREEQYNSTYRVVSVLTVLHQDWLNGKEYKCKVSNKALPAPIEKTISKAKGQPREPQVYVYPP
SRDELTKNQVSLTCLVKGFYPSDIAVEWESNGQPENNYKTTPPVLDSDGSFALVSKLTVDKSRWQQGNVFSCSVMHEALH
NHYTQKSLSLSPGK*GS
SEQ ID NO: 18
EFATMAVMAPRTLVLLLSGALALTQTWAGEVQLVESGGGLVKPGGSLKLSCAASGYTFTSYVMHWVRQAPGKCLEWIGYI
NPYNDGTKYNEKFQGRVTISSDKSISTAYMELSSLRSEDTAMYYCARGTYYYGTRVFDYWGQGTLVTVSSGGGGSGGGGS
GGGGSGGGGSDIVMTQSPATLSLSPGERATLSCRSSKSLQNVNGNTYLYWFQQKPGQSPQLLIYRMSNLNSGVPDRFSGS
GSGTEFTLTISSLEPEDFAVYYCMQHLEYPITFGCGTKLEIKRTVAAPSVFIFPPSDEQLKSGTASVVCLLNNFYPREAK
VQWKVDNALQSGNSQESVTEQDSKDSTYSLSSTLTLSKADYEKHKVYACEVTHQGLSSPVTKSFNRGECGGGGSGGGGSG
GGGSGGGGSGGGGSTKGPSVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALPAPIEKTISKAKGQPRE
PQVYVLPPSRDELTKNQVSLLCLVKGFYPSDIAVEWESNGQPENNYLTWPPVLDSDGSFFLYSKLTVDKSRWQQGNVFSC
SVMHEALHNHYTQKSLSLSPGK*GS
SEQ ID NO: 19
EFATMAVMAPRTLVLLLSGALALTQTWAGQVQLVQSGGGVVQPGRSLRLSCKASGYTFTRYTMHWVRQAPGKCLEWIGYI
NPSRGYTNYNQKVKDRFTISRDNSKNTAFLQMDSLRPEDTGVYFCARYYDDHYCLDYWGQGTPVTVSSGGGGSGGGGSGG
GGSDIQMTQSPSSLSASVGDRVTITCSASSSVSYMNWYQQTPGKAPKRWIYDTSKLASGVPSRFSGSGSGTDYTFTISSL
QPEDIATYYCQQWSSNPFTFGCGTKLQITRTVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKSFNRGECGGGGSGGGGSGGGGSGGGGSGGGG
STKGPSVFPLAPSSKSTSGGTAALGCLVKDYFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYVYPPSRDEL
TKNQVSLTCLVKGFYPSDIAVEWESNGQPENNYKTTPPVLDSDGSFALVSKLTVDKSRWQQGNVFSCSVMHEALHNHYTQ
KSLSLSPGK*GS
SEQ ID NO: 20
EFATMAVMAPRTLVLLLSGALALTQTWAGEVQLVESGGGLVKPGGSLKLSCAASGYTFTSYVMHWVRQAPGKCLEWIGYI
NPYNDGTKYNEKFQGRVTISSDKSISTAYMELSSLRSEDTAMYYCARGTYYYGTRVFDYWGQGTLVTVSSGGGGSGGGGS
GGGGSDIVMTQSPATLSLSPGERATLSCRSSKSLQNVNGNTYLYWFQQKPGQSPQLLIYRMSNLNSGVPDRFSGSGSGTE
FTLTISSLEPEDFAVYYCMQHLEYPITFGCGTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSFNRGECGGGGSGGGGSGGGGSG
GGGSGGGGSTKGPSVFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKCKVSNKALPAPIEKTISKAKGQPREPQVYV
LPPSRDELTKNQVSLLCLVKGFYPSDIAVEWESNGQPENNYLTWPPVLDSDGSFFLYSKLTVDKSRWQQGNVFSCSVMHE
ALHNHYTQKSLSLSPGK*GS
SEQ ID NO: 21
EFATMAVMAPRTLVLLLSGALALTQTWAGEVQLVESGGGLVQPGGSLRLSCAASGFNIKDTYIHWVRQAPGKGLEWVARI
YPTNGYTRYADSVKGRFTISADTSKNTAYLQMNSLRAEDTAVYYCSRWGGDGFYAMDYWGQGTLVTVSSGGGGSGGGGSG
GGGSGGGGSDIQMTQSPSSLSASVGDRVTITCRASQDVNTAVAWYQQKPGKAPKLLIYSASFLYSGVPSRFSGSRSGTDF
TLTISSLQPEDFATYYCQQHYTTPPTFGQGTKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGLSSPVTKSFNRGECGGGGSGGGGSGGGGSGG
GGSGGGGSTKGPSVFPLAPSSKSTSGGTAALGCLVKDYFPEPVTVSWNSGALTSGVHTFPAVLQSSGLYSLSSVVTVPSS
SLGTQTYICNVNHKPSNTKVDKKVEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISKAKGQPREPQVYTY
PPSRDELTKNQVSLTCLVKGFYPSDIAVEWESNGQPENNYKTTPPVLDSDGSFALVSKLTVDKSRWQQGNVFSCSVMHEA
LHNHYTQKSLSLSPGK*GS
SEQ ID NO: 22
EFATMAVMAPRTLVLLLSGALALTQTWAGEVQLVESGGGLVQPGGSLRLSCAASGFNIKDTYIHWVRQAPGKGLEWVARI
YPTNGYTRYADSVKGRFTISADTSKNTAYLQMNSLRAEDTAVYYCSRWGGDGFYAMDYWGQGTLVTVSSGGGGSGGGGSG
GGGSDIQMTQSPSSLSASVGDRVTITCRASQDVNTAVAWYQQKPGKAPKLLIYSASFLYSGVPSRFSGSRSGTDFTLTIS
SLQPEDFATYYCQQHYTTPPTFGQGTKVEIKRTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPVTKSFNRGECGGGGSGGGGSGGGGSGGGGSGG
GGSTKGPSVFPLAPSSKSTSGGTAALGCLVKDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKALPAPIEKTISKAKGQPREPQVYTLPPSRD
ELTKNQVSLLCLVKGFYPSDIAVEWESNGQPENNYMTWPPVLDSDGSFFLYSKLTVDKSRWQQGNVFSCSVMHEALHNHY
TQKSLSLSPGK*GS
SEQ ID NO: 23
EFATMAVMAPRTLVLLLSGALALTQTWAGEVQLVESGGGLVQPGGSLRLSCAASGFNIKDTYIHWVRQAPGKGLEWVARI
YPTNGYTRYADSVKGRFTISADTSKNTAYLQMNSLRAEDTAVYYCSRWGGDGFYAMDYWGQGTLVTVSSGGGGSGGGGSG
GGGSGGGGSDIQMTQSPSSLSASVGDRVTITCRASQDVNTAVAWYQQKPGKAPKLLIYSASFLYSGVPSRFSGSRSGTDF
TLTISSLQPEDFATYYCQQHYTTPPTFGQGTKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGLSSPVTKSFNRGECGGGGSGGGGSGGGGSGG
GGSGGGGSTKGPSVFPLAPSSKSTSGGTAALGCLVKDYFPEPVTVSWNSGALTSGVHTFPAVLQSSGLYSLSSVVTVPSS
SLGTQTYICNVNHKPSNTKVDKKVEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISKAKGQPREPQVYTL
PPSRDELTKNQVSLLCLVKGFYPSDIAVEWESNGQPENNYMTWPPVLDSDGSFFLYSKLTVDKSRWQQGNVFSCSVMHEA
LHNHYTQKSLSLSPGK*GS
SEQ ID NO: 24
EFATMAVMAPRTLVLLLSGALALTQTWAGEVQLVESGGGLVQPGGSLRLSCAASGFNIKDTYIHWVRQAPGKGLEWVARI
YPTNGYTRYADSVKGRFTISADTSKNTAYLQMNSLRAEDTAVYYCSRWGGDGFYAMDYWGQGTLVTVSSGGGGSGGGGSG
GGGSDIQMTQSPSSLSASVGDRVTITCRASQDVNTAVAWYQQKPGKAPKLLIYSASFLYSGVPSRFSGSRSGTDFTLTIS
SLQPEDFATYYCQQHYTTPPTFGQGTKVEIKRTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPVTKSFNRGECGGGGSGGGGSGGGGSGGGGSGG
GGSTKGPSVFPLAPSSKSTSGGTAALGCLVKDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKALPAPIEKTISKAKGQPREPQVYTYPPSRD
ELTKNQVSLTCLVKGFYPSDIAVEWESNGQPENNYKTTPPVLDSDGSFALVSKLTVDKSRWQQGNVFSCSVMHEALHNHY
TQKSLSLSPGK*GS
SEQ ID NO: 25
EFATMAVMAPRTLVLLLSGALALTQTWAGDIQMTQSPSSLSASVGDRVTITCRASQDVNTAVAWYQQKPGKAPKLLIYSA
SFLYSGVPSRFSGSRSGTDFTLTISSLQPEDFATYYCQQHYTTPPTFGQGTKVEIKRTVAAPSVFIFPPSDEQLKSGTAS
VVCLLNNFYPREAKVQWKVDNALQSGNSQESVTEQDSKDSTYSLSSTLTLSKADYEKHKVYACEVTHQGLSSPVTKSFNR
GECGGGGSGGGGSGGGGSGGGGSGGGGSGGGGSGGGGSEVQLVESGGGLVQPGGSLRLSCAASGFNIKDTYIHWVRQAPG
KGLEWVARIYPTNGYTRYADSVKGRFTISADTSKNTAYLQMNSLRAEDTAVYYCSRWGGDGFYAMDYWGQGTLVTVSSAS
TKGPSVFPLAPSSKSTSGGTAALGCLVKDYFPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTISKAKGQPREPQVYTLPPSRDELT
KNQVSLLCLVKGFYPSDIAVEWESNGQPENNYMTWPPVLDSDGSFFLYSKLTVDKSRWQQGNVFSCSVMHEALHNHYTQK
SLSLSPGK*GS
SEQ ID NO: 26
EFATMAVMAPRTLVLLLSGALALTQTWAGEVQLVESGGGLVQPGGSLRLSCAASGFNIKDTYIHWVRQAPGKGLEWVARI
YPTNGYTRYADSVKGRFTISADTSKNTAYLQMNSLRAEDTAVYYCSRWGGDGFYAMDYWGQGTLVTVSSGSTSGSGKPGS
GEGSTKGDIQMTQSPSSLSASVGDRVTITCRASQDVNTAVAWYQQKPGKAPKLLIYSASFLYSGVPSRFSGSRSGTDFTL
TISSLQPEDFATYYCQQHYTTPPTFGQGTKVEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSPVTKSFNRGECGSTSGSTSGSGKPGSGEGS
TKGGSTSSTKGPSVFPLAPSSKSTSGGTAALGCLVKDYFPEPVTVSWNSGALTSGVHTFPAVLQSSGLYSLSSVVTVPSS
SLGTQTYICNVNHKPSNTKVDKKVEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISKAKGQPREPQVYTY
PPSRDELTKNQVSLTCLVKGFYPSDIAVEWESNGQPENNYKTTPPVLDSDGSFALVSKLTVDKSRWQQGNVFSCSVMHEA
LHNHYTQKSLSLSPGK*GS

1. An immunoglobulin construct comprising:
a single chain Fab region (scFab) comprising:
a variable region polypeptide (VH) from an immunoglobulin heavy chain,
a variable region polypeptide (VL) from an immunoglobulin light chain,
a constant region polypeptide (CL) from an immunoglobulin light chain, and
a constant region polypeptide (CH1) from an immunoglobulin heavy chain;
wherein said VH and VL are connected by a first linker to form a single chain Fv construct (scFv).

2. The immunoglobulin construct of claim 1, wherein said CL and CH1 are connected by a second linker.

3. The immunoglobulin construct of claim 2, wherein said single chain Fab region has a sequence comprising VH-L1-VL-CL-L2-CH1, wherein L1 and L2 are first and second linkers.

4. The immunoglobulin construct of claim 2, wherein the single chain Fab region has a sequence comprising VH-L1-VL-L3-CL-L2-CH1, wherein L1, L2 and L3 are linkers.

5. The immunoglobulin construct of claim 2, wherein the single chain Fab region has a sequence comprising VL-L4-VH-CH1-L5-CL, wherein L4 and L5 are linkers.

6. The immunoglobulin construct of claim 2 wherein each linker is a polypeptide comprising from about 1 to about 100 amino acids.

7. The immunoglobulin construct of claim 6, wherein said linker comprises an amino acid sequence comprising amino acids selected from Gly (G), Ser (S) and Glu (E).

8. The immunoglobulin construct of claim 7 wherein said linker is comprised of polypeptide of the general formula (Gly-Gly-Gly-Ser)n wherein n is an integer from 4 to 10.

9. An immunoglobulin construct comprising:
a single chain Fab region (scFab) comprising:
a variable region polypeptide (VH) from an immunoglobulin heavy chain,
a variable region polypeptide (VL) from an immunoglobulin light chain,
a constant region polypeptide (CL) from an immunoglobulin light chain, and
a constant region polypeptide (CH1) from an immunoglobulin heavy chain;
wherein said VH and CL are connected by a linker polypeptide, wherein said linker polypeptide exhibits a propensity to form a helical structure.

10. The immunoglobulin construct of claim 9, wherein said single chain Fab polypeptide has a sequence comprising VL-CL-L8-VH-CH1; wherein L8 is said linker polypeptide.

11. The immunoglobulin construct of claim 9, wherein said linker polypeptide forms at least one of an alpha helix, a polyproline type I helix, a polyproline type II helix and a 3₁₀ helix.

12. The immunoglobulin construct of claim 11, wherein said linker forms between about 1 turn to about 20 turns of a helix.

13. The immunoglobulin construct of claim 9, wherein said linker comprises at least one pair of amino acids that form helix stabilizing interactions.

14. The immunoglobulin construct of claim 13, wherein said helix stabilizing interaction is at least one of a charge-charge interaction, a cation-pi interaction, a hydrophobic interaction and a size complimentary interaction.

15. The immunoglobulin construct of claim 9 wherein said linker polypeptide comprises amino acids selected from Gly (G), Ser (S), Glu (E), Gln (Q), Asp (D), Asn (N), Arg (R), Lys (K), His (H), Val (V) and Ile (I).

16. The immunoglobulin construct of claim 15, wherein said linker has an amino acid sequence comprising at least one (Asp-Asp-Ala-Lys-Lys)n motif wherein n is an integer from 1 to 10.

17. An immunoglobulin construct comprising:
a first polypeptide construct comprising the scFab of claim 1; and a first heavy chain polypeptide comprising a first CH3 region; and
a second polypeptide construct comprising a second heavy chain polypeptide comprising a second CH3 region,
wherein at least one of said first and second heavy chain polypeptides optionally comprises a variant CH3 region that promotes the formation of a heterodimer.

18. The immunoglobulin construct of claim 17, wherein said second polypeptide construct further comprises an antigen binding polypeptide construct.

19. The immunoglobulin construct of claim 18, wherein said antigen binding polypeptide construct is at least one of an scFv or a scFab.

20. The immunoglobulin construct of claim 19, wherein said scFab is the scFab of claim 1.

21. The immunoglobulin construct of claim 17 wherein said first and second heavy chain polypeptides form a heterodimeric Fc.

22. The immunoglobulin construct of claim 21, said heterodimeric Fc comprising a variant immunoglobulin CH3 domain comprising at least one amino acid mutation.

23. The immunoglobulin construct of claim 22, wherein said at least one amino acid mutation promotes the formation of said heterodimeric Fc with stability comparable to a native homodimeric Fc.

24. The immunoglobulin construct according to claim 23, wherein the variant CH3 domain has a melting temperature (Tm) of about 73° C. or greater.

25. The immunoglobulin construct according to claim 23, wherein the heterodimeric Fc is formed with a purity of at least about 70%.

26-27. (canceled)

28. The immunoglobulin construct of claim 17, wherein at least one of said first and second heavy chain polypeptides further comprising a variant CH2 domain comprising amino acid modifications to promote selective binding to at least one of the Fcgamma receptors.

29. The immunoglobulin construct of claim 17, wherein at least one of said first and second heavy chain polypeptides comprises a variant CH2 domain or hinge comprising amino acid modifications that prevents functionally effective binding to at least one of the Fcgamma receptors.

30. The immunoglobulin construct of claim 21 wherein the heterodimeric Fc is glycosylated.

31. The immunoglobulin construct of claim 21 wherein the heterodimeric Fc is aglycosylated.

32. (canceled)

33. The immunoglobulin construct of claim 18, wherein said immunoglobulin construct is bispecific.

34. An immunoglobulin construct comprising:
a first monomeric polypeptide comprising a first single chain Fv polypeptide connected by a linker to a first constant domain polypeptide; and
a second monomeric polypeptide comprising a second single chain Fv polypeptide which is different from said first Fv polypeptide, connected by a linker to a second constant domain polypeptide which is different from said first constant domain polypeptide;
each said constant domain polypeptide comprising at least one each of a CL region, a CH1 region, and a CH3 region or fragments, variants or derivatives thereof; and
wherein said CL and CH1 regions are connected by a linker, and wherein said first and second constant domain polypeptide form a Fc region.

35. The immunoglobulin construct of claim 34 wherein said construct does not contain any CH2 domains.

36. An immunoglobulin construct comprising:
a first monomeric polypeptide comprising a first scFab polypeptide fused to a first constant domain polypeptide; and
a second monomeric polypeptide comprising a second scFab polypeptide which is different from said first Fab polypeptide, fused to a second constant domain polypeptide;
wherein at least one of said first and second scFab polypeptides comprises a linker polypeptide with a propensity to form a helical structure; and
wherein said first and second constant domain polypeptides form a heterodimeric Fc region comprising a variant immunoglobulin CH3 region comprising at least one amino acid mutation that promotes the formation of said heterodimer with stability comparable to a native homodimeric Fc.

37. The immunoglobulin construct of claim 21, wherein said construct can bind at least one cell expressing an antigen, wherein said cell is selected from a list comprising immune cells such as leukocytes, T cells, B cells, Natural Killer cells subendothelial cells, breast, stomach, uterine, nervous, muscle, secretory and reproductive cells.

38-40. (canceled)

41. The isolated immunoglobulin of claim 37, wherein the at least one cell is associated with a disease.

42. The immunoglobulin construct of claim 41 wherein the disease is a cancer selected from a myeloma, a blastoma, a papilloma, an adenoma, a carcinoma, a sarcoma, leukaemia, lymphoma and glioma.

43-44. (canceled)

45. A composition comprising at least one expression vector for expressing the immunoglobulin construct of claim 1, comprising at least one nucleic acid sequence encoding said immunoglobulin construct.

46. A method of producing an expression product containing the immunoglobulin construct of claim 17, in stable mammalian cells, the method comprising: transfecting at least one mammalian cell with:
at least one DNA sequence encoding said immunoglobulin construct to generate stable mammalian cells;
culturing said stable mammalian cells to produce said expression product comprising said immunoglobulin construct.

47. The method of claim 46, wherein said mammalian cell is selected from the group consisting of a VERO, HeLa, HEK, NS0, Chinese Hamster Ovary (CHO), W138, BHK, COS-7, Caco-2 and MDCK cell, and subclasses and variants thereof.

48. A pharmaceutical composition comprising an isolated immunoglobulin construct as defined in claim 17; and a suitable excipient.

49. (canceled)

50. A method of treating cancer in a mammal in need thereof, comprising administering to the mammal a composition comprising an effective amount of the pharmaceutical composition of claims 48.

51-56. (canceled)

57. A kit comprising an immunoglobulin construct as defined in claim 1, and instructions for use thereof.


SEQUENCE LISTING (51 sequences)

SEQ ID NO: 1 - protein, 512 aa, Artificial Sequence
  Description of Artificial Sequence Synthetic polypeptide
EFATMAVMAPRTLVLLLSGALALTQTWAGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSGGGSGSSADDAKKDAAKKDDAKKDDAKKDGGGSGGGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 2 - protein, 514 aa, Artificial Sequence
  Description of Artificial Sequence Synthetic polypeptide
EFATMAVMAPRTLVLLLSGALALTQTWAGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGSTSGSGKPGSGEGSTKGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGSTSGSTSGSGKPGSGEGSTKGGSTSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 3 - protein, 509 aa, Artificial Sequence
  Description of Artificial Sequence Synthetic polypeptide
EFATMAVMAPRTLVLLLSGALALTQTWAGDIQMTQSPSSLSASVGDRVTITCRASRDIKSYLNWYQQKPGKAPKVLIYYATSLAEGVPSRFSGSGSGTDYTLTISSLQPEDFATYYCLQHGESPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSGGGSGSSADDAKKDAAKKDDAKKDDAKKDGGGSGGGSGEVQLVESGGGLVQPGGSLRLSCAASGFNIKEYYMHWVRQAPGKGLEWVGLIDPEQGNTIYDPKFQDRATISADNSKNTAYLQMNSLRAEDTAVYYCARDTAAYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 4 - protein, 733 aa, Artificial Sequence
  Description of Artificial Sequence Synthetic polypeptide
EFATMAVMAPRTLVLLLSGALALTQTWAGDIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGGGSGGGSEGGGSEGGGSEGGGSEGGGSGGGSGEVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 5 - protein, 731 aa, Artificial Sequence
  Description of Artificial Sequence Synthetic polypeptide
EFATMAVMAPRTLVLLLSGALALTQTWAGDIQMTQSPSSLSASVGDRVTITCRASRDIKSYLNWYQQKPGKAPKVLIYYATSLAEGVPSRFSGSGSGTDYTLTISSLQPEDFATYYCLQHGESPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSGGGSGSSADDAKKDAAKKDDAKKDDAKKDGGGSGGGSGEVQLVESGGGLVQPGGSLRLSCAASGFNIKEYYMHWVRQAPGKGLEWVGLIDPEQGNTIYDPKFQDRATISADNSKNTAYLQMNSLRAEDTAVYYCARDTAAYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYMTWPPVLDSDGSFFLYSKLTVDKSRWQQGNVFSCSVMHEALHNHYTQKSLSLSPGK

SEQ ID NO: 6 - protein, 733 aa, Artificial Sequence
  Description of Artificial Sequence Synthetic polypeptide
EFATMAVMAPRTLVLLLSGALALTQTWAGEVQLVESGGGLVQPGGSLRLSCAASGFNIKEYYMHWVRQAPGKGLEWVGLIDPEQGNTIYDPKFQDRATISADNSKNTAYLQMNSLRAEDTAVYYCARDTAAYFDYWGQGTLVTVSSGGGGSGGGGSGGGGSGGGGSDIQMTQSPSSLSASVGDRVTITCRASRDIKSYLNWYQQKPGKAPKVLIYYATSLAEGVPSRFSGSGSGTDYTLTISSLQPEDFATYYCLQHGESPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 7 - protein, 736 aa, Artificial Sequence
  Description of Artificial Sequence Synthetic polypeptide
EFATMAVMAPRTLVLLLSGALALTQTWAGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGSTSGSGKPGSGEGSTKGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGSTSGSTSGSGKPGSGEGSTKGGSTSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYMTWPPVLDSDGSFFLYSKLTVDKSRWQQGNVFSCSVMHEALHNHYTQKSLSLSPGK

SEQ ID NO: 8 - protein, 737 aa, Artificial Sequence
  Description of Artificial Sequence Synthetic polypeptide
EFATMAVMAPRTLVLLLSGALALTQTWAGEVQLVESGGGLVQPGGSLRLSCAASGFNIKEYYMHWVRQAPGKGLEWVGLIDPEQGNTIYDPKFQDRATISADNSKNTAYLQMNSLRAEDTAVYYCARDTAAYFDYWGQGTLVTVSSGGGGSGGGGSGGGGSGGGGSDIQMTQSPSSLSASVGDRVTITCRASRDIKSYLNWYQQKPGKAPKVLIYYATSLAEGVPSRFSGSGSGTDYTLTISSLQPEDFATYYCLQHGESPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYMTWPPVLDSDGSFFLYSKLTVDKSRWQQGNVFSCSVMHEALHNHYTQKSLSLSPGK

SEQ ID NO: 9 - protein, 731 aa, Artificial Sequence
  Description of Artificial Sequence Synthetic polypeptide
EFATMAVMAPRTLVLLLSGALALTQTWAGDIQMTQSPSSLSASVGDRVTITCRASRDIKSYLNWYQQKPGKAPKVLIYYATSLAEGVPSRFSGSGSGTDYTLTISSLQPEDFATYYCLQHGESPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSGGGSGSSADDAKKDAAKKDDAKKDDAKKDGGGSGGGSGEVQLVESGGGLVQPGGSLRLSCAASGFNIKEYYMHWVRQAPGKGLEWVGLIDPEQGNTIYDPKFQDRATISADNSKNTAYLQMNSLRAEDTAVYYCARDTAAYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 10 - protein, 733 aa, Artificial Sequence
  Description of Artificial Sequence Synthetic polypeptide
EFATMAVMAPRTLVLLLSGALALTQTWAGEVQLVESGGGLVQPGGSLRLSCAASGFNIKEYYMHWVRQAPGKGLEWVGLIDPEQGNTIYDPKFQDRATISADNSKNTAYLQMNSLRAEDTAVYYCARDTAAYFDYWGQGTLVTVSSGGGGSGGGGSGGGGSGGGGSDIQMTQSPSSLSASVGDRVTITCRASRDIKSYLNWYQQKPGKAPKVLIYYATSLAEGVPSRFSGSGSGTDYTLTISSLQPEDFATYYCLQHGESPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYMTWPPVLDSDGSFFLYSKLTVDKSRWQQGNVFSCSVMHEALHNHYTQKSLSLSPGK

SEQ ID NO: 11 - protein, 737 aa, Artificial Sequence
  Description of Artificial Sequence Synthetic polypeptide
EFATMAVMAPRTLVLLLSGALALTQTWAGEVQLVESGGGLVQPGGSLRLSCAASGFNIKEYYMHWVRQAPGKGLEWVGLIDPEQGNTIYDPKFQDRATISADNSKNTAYLQMNSLRAEDTAVYYCARDTAAYFDYWGQGTLVTVSSGGGGSGGGGSGGGGSGGGGSDIQMTQSPSSLSASVGDRVTITCRASRDIKSYLNWYQQKPGKAPKVLIYYATSLAEGVPSRFSGSGSGTDYTLTISSLQPEDFATYYCLQHGESPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 12 - protein, 732 aa, Artificial Sequence
  Description of Artificial Sequence Synthetic polypeptide
EFATMAVMAPRTLVLLLSGALALTQTWAGDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSGGGSGSSADDAKKDAAKKDDAKKDDAKKDGGGSGGGSGQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 13 - protein, 740 aa, Artificial Sequence
  Description of Artificial Sequence Synthetic polypeptide
EFATMAVMAPRTLVLLLSGALALTQTWAGDIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSGGGSGSSADDAKKDAAKKDDAKKDDAKKDGGGSGGGSGEVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 14 - protein, 511 aa, Artificial Sequence
  Description of Artificial Sequence Synthetic polypeptide
EFATMAVMAPRTLVLLLSGALALTQTWAGEVQLVESGGGLVQPGGSLRLSCAASGFNIKEYYMHWVRQAPGKGLEWVGLIDPEQGNTIYDPKFQDRATISADNSKNTAYLQMNSLRAEDTAVYYCARDTAAYFDYWGQGTLVTVSSGGGGSGGGGSGGGGSGGGGSDIQMTQSPSSLSASVGDRVTITCRASRDIKSYLNWYQQKPGKAPKVLIYYATSLAEGVPSRFSGSGSGTDYTLTISSLQPEDFATYYCLQHGESPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 15 - protein, 729 aa, Artificial Sequence
  Description of Artificial Sequence Synthetic polypeptide
EFATMAVMAPRTLVLLLSGALALTQTWAGQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 16 - protein, 737 aa, Artificial Sequence
  Description of Artificial Sequence Synthetic polypeptide
EFATMAVMAPRTLVLLLSGALALTQTWAGEVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSGGGGSGGGGSGGGGSDIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 17 - protein, 734 aa, Artificial Sequence
  Description of Artificial Sequence Synthetic polypeptide
EFATMAVMAPRTLVLLLSGALALTQTWAGQVQLVQSGGGVVQPGRSLRLSCKASGYTFTRYTMHWVRQAPGKCLEWIGYINPSRGYTNYNQKVKDRFTISRDNSKNTAFLQMDSLRPEDTGVYFCARYYDDHYCLDYWGQGTPVTVSSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 18 - protein, 742 aa, Artificial Sequence
  Description of Artificial Sequence Synthetic polypeptide
EFATMAVMAPRTLVLLLSGALALTQTWAGEVQLVESGGGLVKPGGSLKLSCAASGYTFTSYVMHWVRQAPGKCLEWIGYINPYNDGTKYNEKFQGRVTISSDKSISTAYMELSSLRSEDTAMYYCARGTYYYGTRVFDYWGQGTLVTVSSGGGGSGGGGSGGGGSGGGGSDIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGCGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 19 - protein, 729 aa, Artificial Sequence
  Description of Artificial Sequence Synthetic polypeptide
EFATMAVMAPRTLVLLLSGALALTQTWAGQVQLVQSGGGVVQPGRSLRLSCKASGYTFTRYTMHWVRQAPGKCLEWIGYINPSRGYTNYNQKVKDRFTISRDNSKNTAFLQMDSLRPEDTGVYFCARYYDDHYCLDYWGQGTPVTVSSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 20 - protein, 737 aa, Artificial Sequence
  Description of Artificial Sequence Synthetic polypeptide
EFATMAVMAPRTLVLLLSGALALTQTWAGEVQLVESGGGLVKPGGSLKLSCAASGYTFTSYVMHWVRQAPGKCLEWIGYINPYNDGTKYNEKFQGRVTISSDKSISTAYMELSSLRSEDTAMYYCARGTYYYGTRVFDYWGQGTLVTVSSGGGGSGGGGSGGGGSDIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGCGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 21 - protein, 736 aa, Artificial Sequence
  Description of Artificial Sequence Synthetic polypeptide
EFATMAVMAPRTLVLLLSGALALTQTWAGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 22 - protein, 731 aa, Artificial Sequence
  Description of Artificial Sequence Synthetic polypeptide
EFATMAVMAPRTLVLLLSGALALTQTWAGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYMTWPPVLDSDGSFFLYSKLTVDKSRWQQGNVFSCSVMHEALHNHYTQKSLSLSPGK

SEQ ID NO: 23 - protein, 736 aa, Artificial Sequence
  Description of Artificial Sequence Synthetic polypeptide
EFATMAVMAPRTLVLLLSGALALTQTWAGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYMTWPPVLDSDGSFFLYSKLTVDKSRWQQGNVFSCSVMHEALHNHYTQKSLSLSPGK

SEQ ID NO: 24 - protein, 731 aa, Artificial Sequence
  Description of Artificial Sequence Synthetic polypeptide
EFATMAVMAPRTLVLLLSGALALTQTWAGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 25 - protein, 728 aa, Artificial Sequence
  Description of Artificial Sequence Synthetic polypeptide
EFATMAVMAPRTLVLLLSGALALTQTWAGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYMTWPPVLDSDGSFFLYSKLTVDKSRWQQGNVFSCSVMHEALHNHYTQKSLSLSPGK

SEQ ID NO: 26 - protein, 736 aa, Artificial Sequence
  Description of Artificial Sequence Synthetic polypeptide
EFATMAVMAPRTLVLLLSGALALTQTWAGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGSTSGSGKPGSGEGSTKGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGSTSGSTSGSGKPGSGEGSTKGGSTSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 27 - protein, 40 aa, Artificial Sequence
  Description of Artificial Sequence Synthetic polypeptide
GGGSGGGSGGGSGGGSGGGSGGGSGGGSGGGSGGGSGGGS

SEQ ID NO: 28 - protein, 50 aa, Artificial Sequence
  Description of Artificial Sequence Synthetic polypeptide
DDAKKDDAKKDDAKKDDAKKDDAKKDDAKKDDAKKDDAKKDDAKKDDAKK

SEQ ID NO: 29 - protein, 8 aa, Artificial Sequence
  Description of Artificial Sequence Synthetic peptide
VSSASTKG

SEQ ID NO: 30 - protein, 80 aa, Artificial Sequence
  Description of Artificial Sequence Synthetic polypeptide
GGGSGGGSGGGSGGGSGGGSGGGSGGGSGGGSGGGSGGGSGGGSGGGSGGGSGGGSGGGSGGGSGGGSGGGSGGGSGGGS

SEQ ID NO: 31 - protein, 17 aa, Artificial Sequence
  Description of Artificial Sequence Synthetic peptide
MLQNSAVLLLLVISASA

SEQ ID NO: 32 - protein, 22 aa, Artificial Sequence
  Description of Artificial Sequence Synthetic peptide
MPTWAWWLFLVLLLALWAPARG

SEQ ID NO: 33 - protein, 42 aa, Artificial Sequence
  Description of Artificial Sequence Synthetic polypeptide
GGGGGSGGGGGSGGGGGSGGGGGSGGGGGSGGGGGSGGGGGS

SEQ ID NO: 34 - protein, 62 aa, Artificial Sequence
  Description of Artificial Sequence Synthetic polypeptide
SSSGGGSSSGGGSSSGGGSSSGGGSEGGGSEGGGSEGGGSEGGGSSSGGGSSSGGGSSSGGG

SEQ ID NO: 35 - protein, 32 aa, Artificial Sequence
  Description of Artificial Sequence Synthetic polypeptide
GSTSGSGGSTSGSGKPGSGEGSTKGGSTSGSG

SEQ ID NO: 36 - protein, 41 aa, Artificial Sequence
  Description of Artificial Sequence Synthetic polypeptide
GGSGGGSGSSADDAKKDAAKKDDAKKDDAKKDGGGSGGGSG

SEQ ID NO: 37 - protein, 30 aa, Artificial Sequence
  Description of Artificial Sequence Synthetic polypeptide
GGGGGSGGGGGSGGGGGSGGGGGSGGGGGS

SEQ ID NO: 38 - protein, 26 aa, Artificial Sequence
  Description of Artificial Sequence Synthetic peptide
GGSSGSTSGSGKPGSGEGSTKGGGSS

SEQ ID NO: 39 - protein, 25 aa, Artificial Sequence
  Description of Artificial Sequence Synthetic peptide
MAVMAPRTLVLLLSGALALTQTWAG

SEQ ID NO: 40 - protein, 30 aa, Artificial Sequence
  Description of Artificial Sequence Synthetic polypeptide
GGGGSGGGGSGGGGSGGGGSGGGGSGGGGS

SEQ ID NO: 41 - protein, 35 aa, Artificial Sequence
  Description of Artificial Sequence Synthetic polypeptide
GGGGSGGGGSGGGGSGGGGSGGGGSGGGGSGGGGS

SEQ ID NO: 42 - protein, 30 aa, Artificial Sequence
  Description of Artificial Sequence Synthetic polypeptide
SGGGSGGGSEGGGSEGGGSEGGGSEGGGSG

SEQ ID NO: 43 - protein, 34 aa, Artificial Sequence
  Description of Artificial Sequence Synthetic polypeptide
SGGGSGGGSEGGGSEGGGSEGGGSEGGGSGGGSG

SEQ ID NO: 44 - protein, 15 aa, Artificial Sequence
  Description of Artificial Sequence Synthetic peptide
GGGGSGGGGSGGGGS

SEQ ID NO: 45 - protein, 20 aa, Artificial Sequence
  Description of Artificial Sequence Synthetic peptide
GGGGSGGGGSGGGGSGGGGS

SEQ ID NO: 46 - protein, 24 aa, Artificial Sequence
  Description of Artificial Sequence Synthetic peptide
GGGGSGGGGSGGGGSGGGGSGGGG

SEQ ID NO: 47 - protein, 28 aa, Artificial Sequence
  Description of Artificial Sequence Synthetic peptide
GGGGSGGGGSGGGGSGGGGSGGGGSGGG

SEQ ID NO: 48 - protein, 18 aa, Artificial Sequence
  Description of Artificial Sequence Synthetic peptide
GSTSGSGKPGSGEGSTKG

SEQ ID NO: 49 - protein, 20 aa, Artificial Sequence
  Description of Artificial Sequence Synthetic peptide
GSTSGSGKPGSGEGSTKGSG

SEQ ID NO: 50 - protein, 26 aa, Artificial Sequence
  Description of Artificial Sequence Synthetic peptide
GSTSGSTSGSGKPGSGEGSTKGGSTS

SEQ ID NO: 51 - protein, 29 aa, Artificial Sequence
  Description of Artificial Sequence Synthetic peptide
EFATMAVMAPRTLVLLLSGALALTQTWAG